(19) United States
(12) Patent Application Publication
(10) Pub. No.: US 2009/0203737 A1
(43) Pub. Date: Aug. 13, 2009

(54) Pyrrole Derivatives as Positive Allosteric Modulators of Metabotropic Receptors

(76) Inventors: Stefania Gagliardi, Milan (IT); Emmanuel Le Poul, Geneva (CH); Iain Lingard, Milan (IT); Giovanni Palombi, Milan (IT); Sonia-Maria Poli, Onex (CH); Jean-Philippe Rocher, Plan-les-Ouates (CH)

Correspondence Address: EDWARDS ANGELL PALMER & DODGE LLP, P.O. BOX 55874, BOSTON, MA, 02205, US

(21) Appl. No.: 11/920,490

(22) PCT Filed: May 17, 2006
(86) PCT No.: PCT/IB2006/002047

§ 371 Date: Jan. 28, 2009

(30) Foreign Application Priority Data
May 18, 2005 (GB) 0510141.5

(51) Int. Cl. A61K 31/4545; C07D 413/14; A61K 31/454; A61P 25/24

(52) U.S. Cl. 514/318; 546/209; 514/326; 514/323; 546/201; 546/193

(57) ABSTRACT

The present invention relates to new compounds which are Pyrrole derivatives of formula (I) wherein A, B, P, Q₅W, R₁ and R₂ are defined in the description. Invention compounds are useful in the prevention or treatment of central or peripheral nervous system disorders as well as other disorders modulated by mGluR5 receptors.

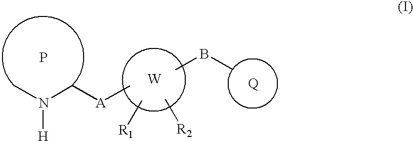

### FIELD OF THE INVENTION

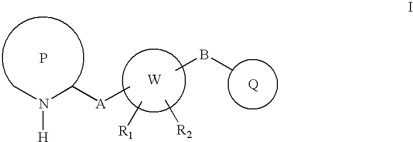

The present invention provides new compounds of formula I as positive allosteric modulators of metabotropic receptors—subtype 5 (“mGluR5”) which are useful for the treatment or prevention of central nervous system disorders such as for example, cognitive decline, both positive and negative symptoms in schizophrenia as well as other central or peripheral nervous system disorders in which the mGluR5 subtype of glutamate metabotropic receptor is involved. The invention is also directed to pharmaceutical compounds and compositions in the prevention or treatment of such diseases in which mGluR5 is involved.

### BACKGROUND OF THE INVENTION

Glutamate, the major amino-acid transmitter in the mammalian central nervous system (CNS), mediates excitatory synaptic neurotransmission through the activation of ionotropic glutamate receptors receptor-channels (iGluRs, namely NMDA, AMPA and kainate) and metabotropic glutamate receptors (mGluRs). iGluRs are responsible for fast excitatory transmission (Nakanishi S et al., (1998) Brain Res Brain Res Rev., 26:230-235) while mGluRs have a more modulatory role that contributes to the fine-tuning of synaptic efficacy. Glutamate performs numerous physiological functions such as long-term potentiation (LTP), a process believed to underlie learning and memory but also cardiovascular regulation, sensory perception, and the development of synaptic plasticity. In addition, glutamate plays an important role in the patho-physiology of different neurological and psychiatric diseases, especially when an imbalance in glutamatergic neurotransmission occurs.

The mGluRs are seven-transmembrane G protein-coupled receptors. The eight members of the family are classified into three groups (Groups I, II & III) according to their sequence homology and pharmacological properties (Schoepp D D et al. (1999) Neuropharmacology, 38:1431-1476). Activation of mGluRs lead to a large variety of intracellular responses and activation of different transductional cascades. Among mGluR members, the mGluR5 subtype is of high interest for counterbalancing the deficit or excesses of neurotransmission in neuropsychiatric diseases. mGluR5 belongs to Group I and its activation initiates cellular responses through G-protein mediated mechanisms. mGluR5 is coupled to phospholipase C and stimulates phosphoinositide hydrolysis and intracellular calcium mobilization.

mGluR5 proteins have been demonstrated to be localized in post-synaptic elements adjacent to the post-synaptic density (Lujan R et al. (1996) Eur J. Neurosci. 8:1488-500; Lujan R et al. (1997) J Chem. Neuroanat., 13:219-41) and are rarely detected in the pre-synaptic elements (Romano C et al. (1995) J Comp Neurol. 355:455-69). mGluR5 receptors can therefore modify the post-synaptic responses to neurotransmitter or regulate neurotransmitter release.

In the CNS, mGluR5 receptors are abundant mainly throughout the cortex, hippocampus, caudate-putamen and nucleus accumbens. As these brain areas have been shown to be involved in emotion, motivational processes and in numerous aspects of cognitive function, mGluR5 modulators are predicted to be of therapeutic interest.

A variety of potential clinical indications have been suggested to be targets for the development of subtype selective mGluR modulators. These include epilepsy, neuropathic and inflammatory pain, numerous psychiatric disorders (eg anxiety and schizophrenia), movement disorders (eg Parkinson disease), neuroprotection (stroke and head injury), migraine and addiction/drug dependency (for reviews, see Brauner-Osborne H et al. (2000) J Med. Chem. 43:2609-45; Bordi F and Ugolini A. (1999) Prog Neurobiol. 59:55-79; Spooren W et al. (2003) Behav Pharmacol: 14:257-77).

The hypothesis of hypofunction of the glutamatergic system as reflected by NMDA receptor hypofunction as a putative cause of schizophrenia has received increasing support over the past few years (Goff D C and Coyle J T (2001) Am J Psychiatry, 158:1367-1377; Carlsson A et al. (2001) Annu Rev Pharmacol Toxicol., 41:237-260 for a review). Evidence implicating dysfunction of glutamatergic neurotransmission is supported by the finding that antagonists of the NMDA subtype of glutamate receptor can reproduce the full range of symptoms as well as the physiologic manifestation of schizophrenia such as hypofrontality, impaired prepulse inhibition and enhanced subcortical dopamine release. In addition, clinical studies have suggested that mGluR5 allele frequency is associated with schizophrenia among certain cohorts (Devon R S et al. (2001) Mol Psychiatry. 6:311-4) and that an increase in mGluR5 message has been found in cortical pyramidal cells layers of schizophrenic brain (Ohnuma T et al. (1998) Brain Res Mol Brain Res. 56:207-17).

The involvement of mGluR5 in neurological and psychiatric disorders is supported by evidence showing that in vivo activation of group I mGluRs induces a potentiation of NMDA receptor function in a variety of brain regions mainly through the activation of mGluR5 receptors (Mannaioni G et al. (2001) Neurosci. 21:5925-34; Awad H et al. (2000) J Neurosci 20:7871-7879; Pisani A et al (2001) Neuroscience 106:579-87; Benquet P et al (2002) J Neurosci. 22:9679-86).

The role of glutamate in memory processes also has been firmly established during the past decade (Martin S J et al. (2000) Annu. Rev. Neurosci. 23:649-711; Baudry M and Lynch G. (2001) Neurobiol Learn Mem., 76:284-297). The use of mGluR5 null mutant mice have strongly supported a role of mGluR5 in learning and memory. These mice show a selective loss in two tasks of spatial learning and memory, and reduced CA1 LTP (Lu et al. (1997) J. Neurosci., 17:5196-5205; Schulz B et al. (2001) Neuropharmacology. 41:1-7; Jia Z et al. (2001) Physiol Behav., 73:793-802; Rodrigues et al. (2002) J Neurosci., 22:5219-5229).

The finding that mGluR5 is responsible for the potentiation of NMDA receptor mediated currents raises the possibility that agonists of this receptor could be useful as cognitive-enhancing agents, but also as novel antipsychotic agents that act by selectively enhancing NMDA receptor function.

The activation of NMDARs could potentiate hypofunctional NMDARs in neuronal circuitry relevant to schizophrenia. Recent in vivo data strongly suggest that mGluR5 activation may be a novel and efficacious approach to treat cognitive decline and both positive and negative symptoms in schizophrenia (Kinney G G et al. (2002) 43:292).

mGluR5 receptor is therefore being considered as a potential drug target for treatment of psychiatric and neurological disorders including treatable diseases in this connection are Anxiety Disorders, Attentional disorders, Eating Disorders, Mood Disorders, Psychotic Disorders, Cognitive Disorders, Personality Disorders and Substance-related disorders.

Most of the current modulators of mGluR5 function have been developed as structural analogues of glutamate, quisqualate or phenylglycine (Schoepp D D et al. (1999) Neuropharmacology, 38:1431-1476) and it has been very challenging to develop in vivo active and selective mGluR5 modulators acting at the glutamate binding site. A new avenue for developing selective modulators is to identify molecules that act through allosteric mechanisms, modulating the receptor by binding to site different from the highly conserved orthosteric binding site.

Positive allosteric modulators of mGluRs have emerged recently as novel pharmacological entities offering this attractive alternative. This type of molecule has been discovered for mGluR1, mGluR2, mGluR4, and mGluR5 (Knoflach F et al. (2001) Proc Natl Acad Sci USA. 98:13402-13407; O'Brien J A et al. (2003) Mol Pharmacol. 64:731-40; Johnson K et al. (2002) Neuropharmacology 43:291; Johnson M P et al. (2003) J Med Chem. 46:3189-92; Marino M J et al. (2003) Proc Natl Acad Sci USA. 100 (23):13668-73; for a review see Mutel V (2002) Expert Opin. Ther. Patents 12:1-8; Kew J N (2004) Pharmacol Ther. 104 (3):233-44; Johnson M P et al (2004) Biochem Soc Trans. 32:881-7). DFB and related molecules were described as in vitro mGluR5 positive allosteric modulators but with low potency (O'Brien J A et al. (2003) Mol. Pharmacol. 64:731-40). Benzamide derivatives have been patented (WO 2004/087048; O'Brien J A (2004) J. Pharmacol. Exp. Ther. 309:568-77) and recently aminopyrazole derivatives have been disclosed as mGluR5 positive allosteric modulators (Lindsley et al. (2004) J. Med. Chem. 47:5825-8; WO 2005/087048). Among aminopyrazole derivatives, CDPPB has shown in vivo activity antipsychotic-like effects in rat behavioral models (Kinney G G et al. (2005) J Pharmacol Exp Ther 313:199-206). This report is consistent with the hypothesis that allosteric potentiation of mGluR5 may provide a novel approach for development of antipsychotic agents. Recently a novel series of positive allosteric modulators of mGluR5 receptors has been disclosed (WO 2005/044797). International publication WO 99/45006 by Pfizer Inc. discloses oxadiazolyl piperidine derivatives as rotamase enzyme inhibitors. Several classes of aryl and heteroaryloxadiazole compounds have been disclosed: U.S. Ser. No. 04/106607, WO 03/056823, WO 02/72570, GB 1164572, FR 6671).

None of the specifically disclosed compounds are structurally related to the compounds of the present invention.

The present invention relates to a method of treating or preventing a condition in a mammal, including a human, the treatment or prevention of which is affected or facilitated by the neuromodulatory effect of mGluR5 positive allosteric modulators.

### FIGURES

FIG. 1 shows the effect of 10 μM of the example #1 of the present invention on primary cortical mGluR5-expressing cell cultures in the absence or in the presence of 300 nM glutamate.

### DETAILED DESCRIPTION OF THE INVENTION

According to the present invention, there are provided new compounds of the general formula I

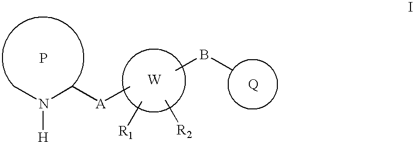

Or pharmaceutically acceptable salts, hydrates or solvates of such compounds
Wherein
W represents (C₄-C₇)cycloalkyl, (C₃-C₇)heterocycloalkyl, (C₃-C₇)heterocycloalkyl-(C₁-C₃)alkyl or (C₃-C₇)heterocycloalkenyl ring;
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula

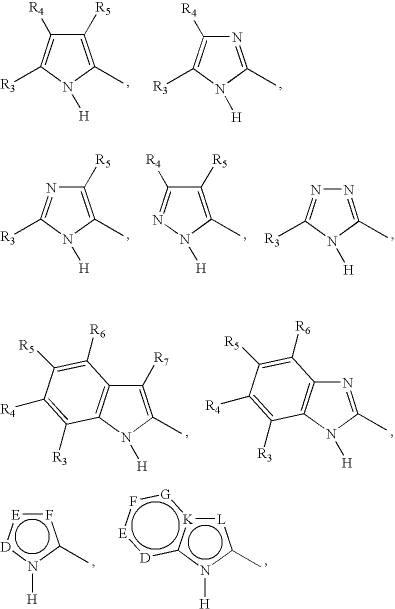

R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula

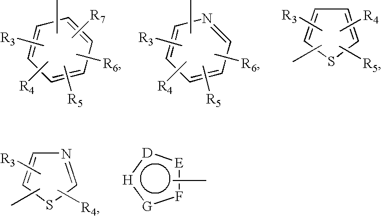

R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
A is azo —N═N—, ethyl, ethenyl, ethynyl, —NR₈C(═O)—, —NR₈C(═O)—O—, —NR₈C(═O)—NR₉, NR₈S(═O)₂—, —C(═O)NR₈—, —O—C(═O)NR₈—, —S—, —S(═O)—, —S(═O)₂—, —S(═O)₂NR₈—, —C(═O)—O—, —O—C(═O)—, —C(═NR₈)NR₉—, C(═NOR₈)NR₉—, —NR₈C(═NOR₉)—, ═N—O—, —O—N═CH— or a group aryl or heteroaryl of formula

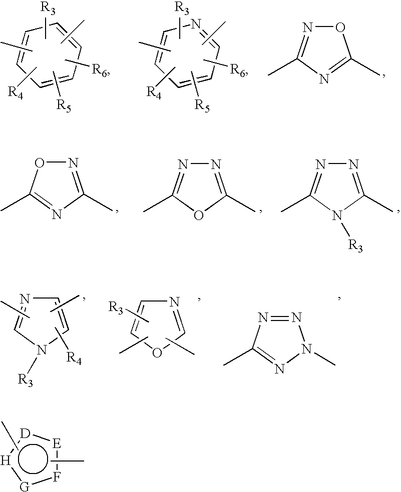

R₃, R₄, R₅ and R₆ independently are as defined above;
D, E, F, G and H in A independently represent —C(R₃)═, —C(R₃)═C(R₄)—, —C(═O)—, —C(═S)—, —O—, —N═, —N(R₃)— or —S—; R₃, R₄, R₅ and R₆ independently are as defined above;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
   Any N may be an N-oxide.

The present invention includes both possible stereoisomers and includes not only racemic compounds but the individual enantiomers as well.

For the avoidance of doubt it is to be understood that in this specification “(C₁-C₆)” means a carbon group having 1, 2, 3, 4, 5 or 6 carbon atoms. “(C₀-C₆)” means a carbon group having 0, 1, 2, 3, 4, 5 or 6 carbon atoms.

In this specification “C” means a carbon atom.

In the above definition, the term “(C₁-C₆)alkyl” includes group such as methyl, ethyl, propyl, isopropyl, butyl, isobutyl, sec-butyl, tert-butyl, pentyl, isopentyl, neopentyl, tert-pentyl, hexyl or the like.

“(C₂-C₆)alkenyl” includes group such as ethenyl, 1-propenyl, allyl, isopropenyl, 1-butenyl, 3-butenyl, 4-pentenyl and the like.

“(C₂-C₆)alkynyl” includes group such as ethynyl, propynyl, butynyl, pentynyl and the like.

“Halogen” includes atoms such as fluorine, chlorine, bromine and iodine.

“Cycloalkyl” refers to an optionally substituted carbocycle containing no heteroatoms, includes mono-, bi-, and tricyclic saturated carbocycles, as well as fused ring systems. Such fused ring systems can include on ring that is partially or fully unsaturated such as a benzene ring to form fused ring systems such as benzo fused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, fluorenyl, 1,2,3,4-tetrahydronaphthalene and the like.

“Heterocycloalkyl” refers to an optionally substituted carbocycle containing at least one heteroatom selected independently from O, N, S. It includes mono-, bi-, and tricyclic saturated carbocycles, as well as fused ring systems. Such fused ring systems can include one ring that is partially or fully unsaturated such as a benzene ring to form fused ring systems such as benzo fused carbocycles. Examples of heterocycloalkyl include piperidine, piperazine, morpholine, tetrahydrothiophene, indoline, isoquinoline and the like.

“Aryl” includes (C₆-C₁₀)aryl group such as phenyl, 1-naphtyl, 2-naphtyl and the like.

“Arylalkyl” includes (C₆-C₁₀)aryl-(C₁-C₃)alkyl group such as benzyl group, 1-phenylethyl group, 2-phenylethyl group, 1-phenylpropyl group, 2-phenylpropyl group, 3-phenylpropyl group, 1-naphtylmethyl group, 2-naphtylmethyl group or the like.

“Heteroaryl” includes 5-10 membered heterocyclic group containing 1 to 4 heteroatoms selected from oxygen, nitrogen or sulphur to form a ring such as furyl (furan ring), benzofuranyl (benzofuran ring), thienyl (thiophene ring), benzothiophenyl (benzothiophene ring), pyrrolyl (pyrrole ring), imidazolyl (imidazole ring), pyrazolyl (pyrazole ring), thiazolyl (thiazole ring), isothiazolyl (isothiazole ring), triazolyl (triazole ring), tetrazolyl (tetrazole ring), pyridil (pyridine ring), pyrazynyl (pyrazine ring), pyrimidinyl (pyrimidine ring), pyridazinyl (pyridazine ring), indolyl (indole ring), isoindolyl (isoindole ring), benzoimidazolyl (benzimidazole ring), purinyl group (purine ring), quinolyl (quinoline ring), phtalazinyl (phtalazine ring), naphtyridinyl (naphtyridine ring), quinoxalinyl (quinoxaline ring), cinnolyl (cinnoline ring), pteridinyl (pteridine ring), oxazolyl (oxazole ring), isoxazolyl (isoxazole ring), benzoxazolyl (benzoxazole ring), benzothiazolyl (benzothiaziole ring), furazanyl (furazan ring) and the like.

“Heteroarylalkyl” includes heteroaryl-(C₁-C₃-alkyl) group, wherein examples of heteroaryl are the same as those illustrated in the above definition, such as 2-furylmethyl group, 3-furylmethyl group, 2-thienylmethyl group, 3-thienylmethyl group, 1-imidazolylmethyl group, 2-imidazolylmethyl group, 2-thiazolylmethyl group, 2-pyridylmethyl group, 3-pyridylmethyl group, 1-quinolylmethyl group or the like.

“Solvate” refers to a complex of variable stoichiometry formed by a solute (e.g. a compound of formula I) and a solvent. The solvent is a pharmaceutically acceptable solvent as water preferably; such solvent may not interfere with the biological activity of the solute.

“Optionally” means that the subsequently described event(s) may or may not occur, and includes both event(s), which occur, and events that do not occur.

The term “substituted” refers to substitution with the named substituent or substituents, multiple degrees of substitution being allowed unless otherwise stated.

Preferred compounds of the present invention are compounds of formula I-A depicted below

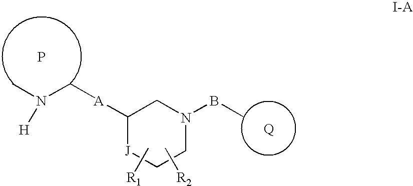

or pharmaceutically acceptable salts, hydrates or solvates of such compounds.
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula

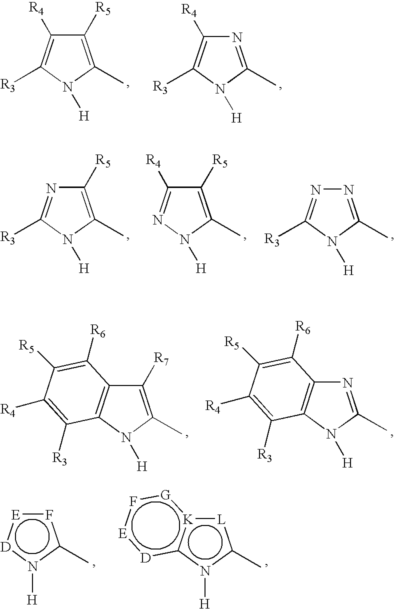

R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula

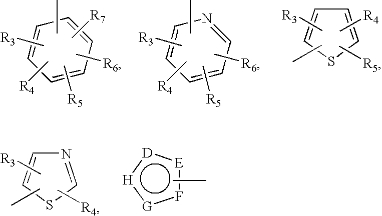

R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
A is azo —N═N—, ethyl, ethenyl, ethynyl, —NR₈C(═O)—, —NR₈C(═O)—O—, —NR₈C(═O)—NR₉, NR₈S(═O)₂—, —C(═O)NR₈—, —O—C(═O)NR₈—, —S—, —S(═O)—, —S(═O)₂—, —S(═O)₂NR₈—, —C(═O)—O—, —O—C(═O)—, —C(═NR₈)NR₉—, C(═NOR₈)NR₉—, —NR₈C(═NOR₉)—, ═N—O—, —O—N═CH— or a group aryl or heteroaryl of formula

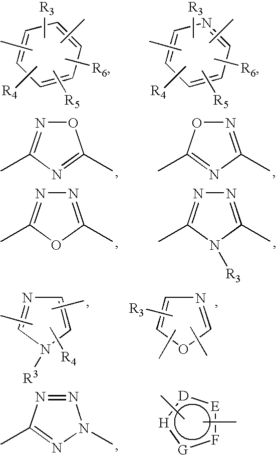

R₃, R₄, R₅ and R₆ independently are as defined above;
D, E, F, G and H in A independently represent —C(R₃)═, —C(R₃)═C(R₄)—, —C(═O)—, —C(═S)—, —O—, —N═, —N(R₃)— or —S—; R₃, R₄, R₅ and R₆ independently are as defined above;
B represents a single bond, —C(═O)—(C₀-C₂)alkyl-, —C(—O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
J represents a single bond, —C(R₁₀, R₁₁), —O—, —N(R₁₀)— or —S—;
   R₁₀, R₁₁ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

Particularly preferred compounds of the present invention are compounds of formula I-B

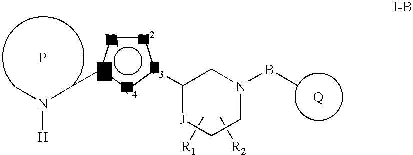

Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula

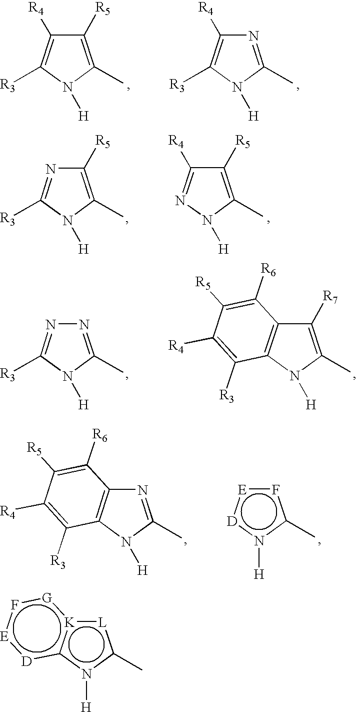

R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(—NOR)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇-)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula

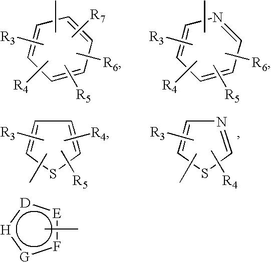

R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
V₁, V₂, V₃, V₄ and V₅ represent independently —C(R₃)═, —C(R₃)═C(R₄)—, —C(═O)—, —C(═S)—, —O—, —N═, —N(R₃)— or —S—;
B represents a single bond, —C(═O—-(C₀-C₂)alkyl-, —C(—O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R and R₉, independently are as defined above;
J represents a single bond, —C(R₁₀, R₁₁), —O—, —N(R₁₀)— or —S—;
   R₁₀, R₁₁ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

Further preferred compounds of the present invention are compounds of formula I-C

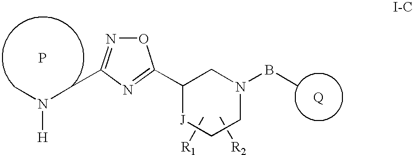

or pharmaceutically acceptable salts, hydrates or solvates of such compounds.
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula

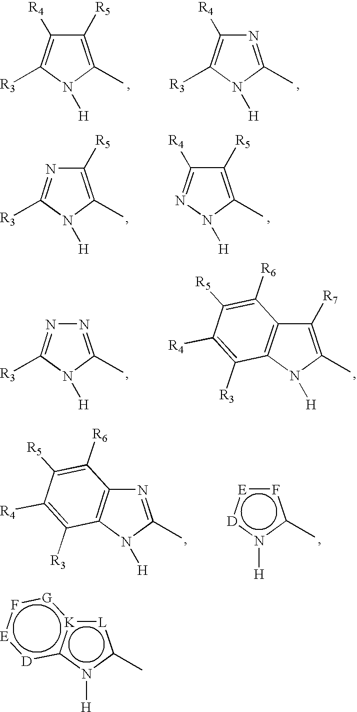

R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(—O)R₈, —C(O)—O—R₈, —C(═O)NR₈R₉, —C(—NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula

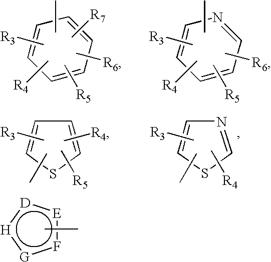

R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(—O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
J represents a single bond, —C(R₁₀, R₁₁), —O—, —N(R₁₀)— or —S—;
   R₁₀, R₁₁ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

In another aspect, the compound of this invention is represented by formula (I-D) or a pharmaceutically acceptable salt thereof

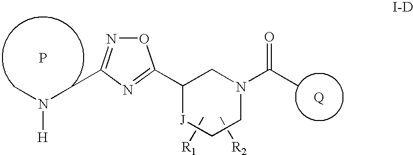

or pharmaceutically acceptable salts, hydrates or solvates of such compounds.
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula

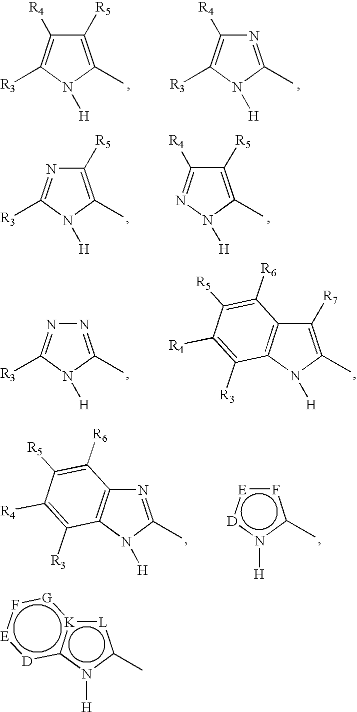

R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, -heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(—S)—, —O—, —N═, —N(R₃)— or —S—;
Q denotes a cycloalkyl, an aryl or heteroaryl group of formula

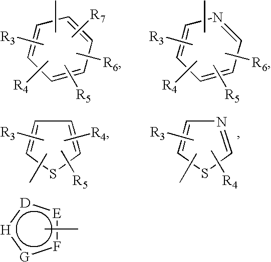

R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
J represents a single bond, —C(R₁₀, R₁₁), —O—, —N(R₁₀)— or —S—;
   R₁₀, R₁₁, independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

Another aspect of the invention are compounds of the formula II-A

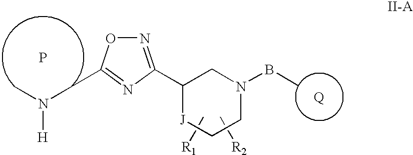

or pharmaceutically acceptable salts, hydrates or solvates of such compounds.
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula

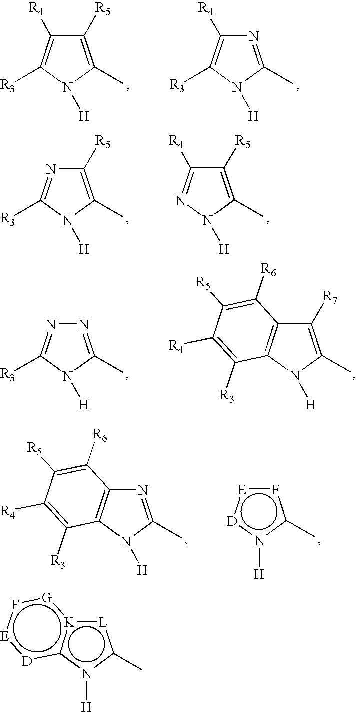

R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(—NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula

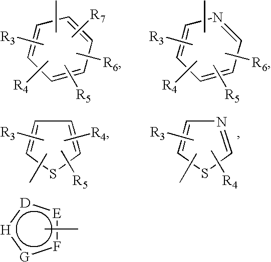

R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
   R₈ and R₉, independently are as defined above;
J represents a single bond, —C(R₁₀, R₁₁), —O—, —N(R₁₀)— or —S—;
   R₁₀, R₁₁ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

An embodiment of the present invention includes compounds of the formula II-B

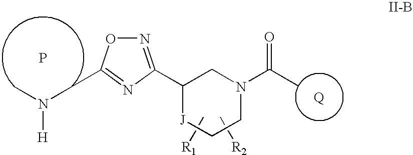

or pharmaceutically acceptable salts, hydrates or solvates of such compounds.
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula

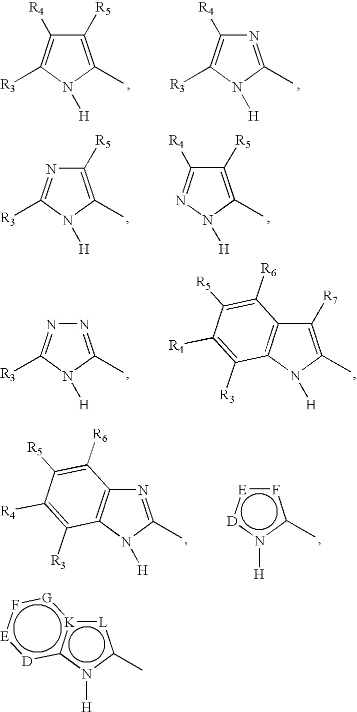

R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(—NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃-)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula

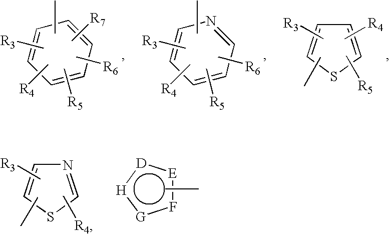

R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
J represents a single bond, —C(R₁₀, R₁₁), —O—, —N(R₁₀)— or —S—;
   R₁₀, R₁₁, independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
   Any N may be an N-oxide;

The present invention includes both possible stereoisomers and includes not only racemic compounds but the individual enantiomers as well.

Specifically preferred compounds are:
(4-Fluoro-phenyl)-{(S)-3-[3-(1H-pyrrol-2-yl)-[1,2,4]oxadiazol-5-yl]-piperidin-1-yl}-methanone
(2,4-Difluoro-phenyl)-{(S)-3-[3-(1H-pyrrol-2-yl)-[1,2,4]oxadiazol-5-yl]-piperidin-1-yl}-methanone
(3,4-Difluoro-phenyl)-{(S)-3-[3-(1H-pyrrol-2-yl)-[1,2,4]oxadiazol-5-yl]-piperidin-1-yl}-methanone
(6-Fluoro-pyridin-3-yl)-{(S)-3-[3-(1H-pyrrol-2-yl)-[1,2,4]oxadiazol-5-yl]-piperidin-1-yl}-methanone
(3,4-Difluoro-phenyl)-{3-[5-(1H-pyrrol-2-yl)-[1,2,4]oxadiazol-3-yl]-piperidin-1-yl}-methanone
(2,4-Difluoro-phenyl)-{3-[5-(1H-pyrrol-2-yl)-[1,2,4]oxadiazol-3-yl]-piperidin-1-yl}-methanone
(4-Fluoro-phenyl)-{3-[5-(1H-pyrrol-2-yl)-[1,2,4]oxadiazol-3-yl]-piperidin-1-yl}-methanone
(6-Fluoro-pyridin-3-yl)-{3-[5-(1H-pyrrol-2-yl)-[1,2,4]oxadiazol-3-yl]-piperidin-1-yl}-methanone
(4-Fluoro-2-methyl-phenyl)-{3-[5-(1H-pyrrol-2-yl)-[1,2,4]oxadiazol-3-yl]-piperidin-1-yl}-methanone
(3,4-Difluoro-phenyl)-{(S)-3-[5-(1H-pyrrol-2-yl)-[1,2,4]oxadiazol-3-yl]-piperidin-1-yl}-methanone
(4-Fluoro-phenyl)-{3-[5-(1H-indol-2-yl)-[1,2,4]oxadiazol-3-yl]-piperidin-1-yl}-methanone
(2,4-Difluoro-phenyl)-{(S)-3-[3-(1H-indol-2-yl)-[1,2,4]oxadiazol-5-yl]-piperidin-1-yl}-methanone
(4-Fluoro-phenyl)-{3-[5-(2H-pyrazol-3-yl)-[1,2,4]oxadiazol-3-yl]-piperidin-1-yl}-methanone
(3,4-Difluoro-phenyl)-{3-[5-(2H-pyrazol-3-yl)-[1,2,4]oxadiazol-3-yl]-piperidin-1-yl}-methanone
(4-Fluoro-phenyl)-{3-[5-(1H-imidazol-2-yl)-[1,2,4]oxadiazol-3-yl]-piperidin-1-yl}-methanone
(3,4-Difluoro-phenyl)-{(S)-3-[3-(1H-indol-2-yl)-[1,2,4]oxadiazol-5-yl]-piperidin-1-yl}-methanone
(4-Fluoro-phenyl)-{(S)-3-[3-(1H-indol-2-yl)-[1,2,4]oxadiazol-5-yl]-piperidin-1-yl}-methanone
(3,4-diFluoro-phenyl)-{3-[5-(1H-imidazol-2-yl)-[1,2,4]oxadiazol-3-yl]-piperidin-1-yl}-methanone.
{(S)-3-[3-(1H-Indol-2-yl)-[1,2,4]oxadiazol-5-yl]-piperidin-1-yl}-(5-methyl-isoxazol-4-yl)-methanone
(5-Methyl-isoxazol-4-yl)-{(S)-3-[3-(1H-pyrrol-2-yl)-[1,2,4]oxadiazol-5-yl]-piperidin-1-yl}-methanone
(6-Fluoro-pyridin-3-yl)-{(S)-3-[5-(1H-pyrrol-2-yl)-[1,2,4]oxadiazol-3-yl]-piperidin-1-yl}-methanone
(4-Fluoro-phenyl)-{(S)-3-[5-(1H-pyrrol-2-yl)-[1,2,4]oxadiazol-3-yl]-piperidin-1-yl}-methanone
(6-Fluoro-pyridin-3-yl)-{3-[5-(1H-indol-2-yl)-[1,2,4]oxadiazol-3-yl]-piperidin-1-yl}-methanone
(4-Fluoro-phenyl)-{(S)-3-[3-(1H-imidazol-2-yl)-[1,2,4]oxadiazol-5-yl]-piperidin-1-yl}-methanone
(3,4-Difluoro-phenyl)-{(S)-3-[3-(1H-imidazol-2-yl)-[1,2,4]oxadiazol-5-yl]-piperidin-1-yl}-methanone
{3-[5-(1H-Indol-2-yl)-[1,2,4]oxadiazol-3-yl]-piperidin-1-yl}-(5-methyl-isoxazol-4-yl)-methanone
(4-Fluoro-phenyl)-{(S)-3-[5-(4-methyl-1H-pyrrol-2-yl)-[1,2,4]oxadiazol-3-yl]-piperidin-1-yl}-methanone
(6-Fluoro-pyridin-3-yl)-{(S)-3-[5-(4-methyl-1H-pyrrol-2-yl)-[1,2,4]oxadiazol-3-yl]-piperidin-1-yl}-methanone
(5-Methyl-isoxazol-4-yl)-{(S)-3-[5-(4-methyl-1H-pyrrol-2-yl)-[1,2,4]oxadiazol-3-yl]-piperidin-1-yl}-methanone
(2-Fluoro-pyridin-4-yl)-{(S)-3-[5-(4-methyl-1H-pyrrol-2-yl)-[1,2,4]oxadiazol-3-yl]-piperidin-1-yl}-methanone
(4-Fluoro-phenyl)-{(S)-3-[3-(4-methyl-1H-pyrrol-2-yl)-[1,2,4]oxadiazol-5-yl]-piperidin-1-yl}-methanone
(3,4-Difluoro-phenyl)-{(S)-3-[3-(4-methyl-1H-pyrrol-2-yl)-[1,2,4]oxadiazol-5-yl]-piperidin-1-yl}-methanone
(6-Fluoro-pyridin-3-yl)-{(S)-3-[3-(4-methyl-1H-pyrrol-2-yl)-[1,2,4]oxadiazol-5-yl]-piperidin-1-yl}-methanone
(2-Fluoro-pyridin-4-yl)-{(S)-3-[3-(4-methyl-1H-pyrrol-2-yl)-[1,2,4]oxadiazol-5-yl]-piperidin-1-yl}-methanone
(5-Methyl-isoxazol-4-yl)-{(S)-3-[3-(4-methyl-1H-pyrrol-2-yl)-[1,2,4]oxadiazol-5-yl]-piperidin-1-yl}-methanone
(4-Fluoro-phenyl)-{(S)-3-[5-(4-nitro-1H-pyrrol-2-yl)-[1,2,4]oxadiazol-3-yl]-piperidin-1-yl}-methanone
(4-Fluoro-phenyl)-{(R)-3-[3-(1H-pyrrol-2-yl)-[1,2,4]oxadiazol-5-yl]-piperidin-1-yl}-methanone
(4-Fluoro-phenyl)-{(S)-3-[5-(5-methyl-1H-pyrrol-2-yl)-[1,2,4]oxadiazol-3-yl]-piperidin-1-yl}-methanone
{(S)-3-[5-(4-Chloro-1H-pyrrol-2-yl)-[1,2,4]oxadiazol-3-yl]-piperidin-1-yl}-(4-fluoro-phenyl)-methanone
{(S)-3-[5-(4-Chloro-1H-pyrrol-2-yl)-[1,2,4]oxadiazol-3-yl]-piperidin-1-yl}-(6-fluoro-pyridin-3-yl)-methanone
{(S)-3-[5-(4-Chloro-1H-pyrrol-2-yl)-[1,2,4]oxadiazol-3-yl]-piperidin-1-yl}-(2-fluoro-pyridin-4-yl)-methanone
{(S)-3-[5-(4-Chloro-1H-pyrrol-2-yl)-[1,2,4]oxadiazol-3-yl]-piperidin-1-yl}-(5-methyl-isoxazol-4-yl)-methanone
{(S)-3-[3-(4-Chloro-1H-pyrrol-2-yl)-[1,2,4]oxadiazol-5-yl]-piperidin-1-yl}-(4-fluoro-phenyl)-methanone
{(S)-3-[5-(4-Bromo-1H-pyrrol-2-yl)-[1,2,4]oxadiazol-3-yl]-piperidin-1-yl}-(6-fluoro-pyridin-3-yl)-methanone
{(S)-3-[3-(4-Bromo-1H-pyrrol-2-yl)-[1,2,4]oxadiazol-5-yl]-piperidin-1-yl}-(4-fluoro-phenyl)-methanone
{(S)-3-[3-(4-Bromo-1H-pyrrol-2-yl)-[1,2,4]oxadiazol-5-yl]-piperidin-1-yl}-(6-fluoro-pyridin-3-yl)-methanone
(4-Fluoro-phenyl)-{3-fluoro-3-[3-(1H-pyrrol-2-yl)-[1,2,4]oxadiazol-5-yl]-piperidin-1-yl}-methanone
{3,3-Difluoro-5-[3-(1H-pyrrol-2-yl)-[1,2,4]oxadiazol-5-yl]-piperidin-1-yl}-(4-fluoro-phenyl)-methanone
{3,3-Dimethyl-5-[3-(1H-pyrrol-2-yl)-[1,2,4]oxadiazol-5-yl]-piperidin-1-yl}-(4-fluoro-phenyl)-methanone
(4-Fluoro-phenyl)-{(S)-3-[3-(4-fluoro-1H-pyrrol-2-yl)-[1,2,4]oxadiazol-5-yl]-piperidin-1-yl}-methanone
(3,4-Difluoro-phenyl)-{(S)-3-[3-(4-fluoro-1H-pyrrol-2-yl)-[1,2,4]oxadiazol-5-yl]-piperidin-1-yl}-methanone
(6-Fluoro-pyridin-3-yl)-{(S)-3-[3-(4-fluoro-1H-pyrrol-2-yl)-[1,2,4]oxadiazol-5-yl]-piperidin-1-yl}-methanone
(2-Fluoro-pyridin-4-yl)-{(S)-3-[3-(4-fluoro-1H-pyrrol-2-yl)-[1,2,4]oxadiazol-5-yl]-piperidin-1-yl}-methanone
(4-Fluoro-phenyl)-{(S)-3-[5-(1H-pyrrol-2-yl)-tetrazol-2-yl]-piperidin-1-yl}-methanone
(4-Fluoro-phenyl)-{(S)-3-[5-(4-trifluoromethyl-1H-imidazol-2-yl)-[1,2,4]oxadiazol-3-yl]-piperidin-1-yl}-methanone
(6-Fluoro-pyridin-3-yl)-{(S)-3-[5-(4-isopropyl-1H-pyrrol-2-yl)-[1,2,4]oxadiazol-3-yl]-piperidin-1-yl}-methanone
(4-Fluoro-phenyl)-{3-[3-(1H-pyrrol-2-yl)-[1,2,4]oxadiazol-5-yl]-pyrrolidin-1-yl}-methanone
(3-Fluoro-pyridin-4-yl)-{(S)-3-[5-(4-methyl-1H-pyrrol-2-yl)-[1,2,4]oxadiazol-3-yl]-piperidin-1-yl}-methanone
{(S)-3-[5-(4-Chloro-1H-pyrrol-2-yl)-[1,2,4]oxadiazol-3-yl]-piperidin-1-yl}-(3-fluoro-pyridin-4-yl)-methanone
(2-Fluoro-pyridin-4-yl)-{(S)-3-[5-(4-fluoro-1H-pyrrol-2-yl)-[1,2,4]oxadiazol-3-yl]-piperidin-1-yl}-methanone
{(S)-3-[5-(4-Bromo-1H-pyrrol-2-yl)-[1,2,4]oxadiazol-3-yl]-piperidin-1-yl}-(3-fluoro-pyridin-4-yl)-methanone
(3-Fluoro-pyridin-4-yl)-{(S)-3-[5-(4-fluoro-1H-pyrrol-2-yl)-[1,2,4]oxadiazol-3-yl]-piperidin-1-yl}-methanone
(4-Fluoro-phenyl)-{(S)-3-[5-(4-fluoro-1H-pyrrol-2-yl)-[1,2,4]oxadiazol-3-yl]-piperidin-1-yl}-methanone
(6-Fluoro-pyridin-3-yl)-{(S)-3-[5-(4-fluoro-1H-pyrrol-2-yl)-[1,2,4]oxadiazol-3-yl]-piperidin-1-yl}-methanone
{(S)-3-[3-(4-Chloro-1H-pyrrol-2-yl)-[1,2,4]oxadiazol-5-yl]-piperidin-1-yl}-(6-fluoro-pyridin-3-yl)-methanone
{(S)-3-[3-(4-Chloro-1H-pyrrol-2-yl)-[1,2,4]oxadiazol-5-yl]-piperidin-1-yl}-(2-fluoro-pyridin-4-yl)-methanone
{(S)-3-[3-(4-Chloro-1H-pyrrol-2-yl)-[1,2,4]oxadiazol-5-yl]-piperidin-1-yl}-(3-fluoro-pyridin-4-yl)-methanone
{(S)-3-[3-(4-Chloro-1H-pyrrol-2-yl)-[1,2,4]oxadiazol-5-yl]-piperidin-1-yl}-(5-methyl-isoxazol-4-yl)-methanone
{(S)-3-[3-(4-Bromo-1H-pyrrol-2-yl)-[1,2,4]oxadiazol-5-yl]-piperidin-1-yl}-(3-fluoro-pyridin-4-yl)-methanone
(3-Fluoro-pyridin-4-yl)-{(S)-3-[3-(4-fluoro-1H-pyrrol-2-yl)-[1,2,4]oxadiazol-5-yl]-piperidin-1-yl}-methanone
(3-Fluoro-pyridin-4-yl)-{(S)-3-[3-(4-methyl-1H-pyrrol-2-yl)-[1,2,4]oxadiazol-5-yl]-piperidin-1-yl}-methanone
(4-Fluoro-phenyl)-{(S)-3-[5-(4-cyano-1H-pyrrol-2-yl)-[1,2,4]oxadiazol-3-yl]-piperidin-1-yl}-methanone
5-{3-[(S)-1-(6-Fluoro-pyridine-3-carbonyl)-piperidin-3-yl]-[1,2,4]oxadiazol-5-yl}-1H-pyrrole-3-carbonitrile
5-{3-[(S)-1-(2-Fluoro-pyridine-4-carbonyl)-piperidin-3-yl]-[1,2,4]oxadiazol-5-yl}-1H-pyrrole-3-carbonitrile
5-{3-[(S)-1-(3-Fluoro-pyridine-4-carbonyl)-piperidin-3-yl]-[1,2,4]oxadiazol-5-yl}-1H-pyrrole-3-carbonitrile
(4-Fluoro-phenyl)-{(S)-3-[5-(4-trifluoromethyl-1H-pyrrol-2-yl)-[1,2,4]oxadiazol-3-yl]-piperidin-1-yl}-methanone
(3-Fluoro-pyridin-4-yl)-{(S)-3-[5-(4-trifluoromethyl-1H-pyrrol-2-yl)[1,2,4]oxadiazol-3-yl]-piperidin-1-yl}-methanone
(6-Fluoro-pyridin-3-yl)-{(S)-3-[5-(4-trifluoromethyl-1H-pyrrol-2-yl)-[1,2,4]oxadiazol-3-yl]-piperidin-1-yl}-methanone
(3,4-Difluoro-phenyl)-{(S)-3-[3-(4-methyl-1H-imidazol-2-yl)-[1,2,4]oxadiazol-5-yl]-piperidin-1-yl}-methanone
{(S)-3-[5-(4-Chloro-1H-pyrrol-2-yl)-[1,2,4]oxadiazol-3-yl]-piperidin-1-yl}-pyridin-4-yl-methanone
(6-Fluoro-pyridin-3-yl)-{(S)-3-[3-(4-trifluoromethyl-1H-pyrrol-2-yl)-[1,2,4]oxadiazol-5-yl]-piperidin-1-yl}-methanone.

The present invention relates to the pharmaceutically acceptable acid addition salts of compounds of the formula I or pharmaceutically acceptable carriers or excipients.

The present invention relates to a method of treating or preventing a condition in a mammal, including a human, the treatment or prevention of which is affected or facilitated by the neuromodulatory effect of mGluR5 allosteric modulators and particularly positive allosteric modulators.

The present invention relates to a method useful for treating or preventing peripheral and central nervous system disorders such as tolerance or dependence, anxiety, depression, psychiatric disease such as psychosis, inflammatory or neuropathic pain, memory impairment, Alzheimer's disease, ischemia, drug abuse and addiction.

The present invention relates to pharmaceutical compositions which provide from about 0.01 to 1000 mg of the active ingredient per unit dose. The compositions may be administered by any suitable route. For example orally in the form of capsules, parenterally in the form of solutions for injection, topically in the form of unguents or lotions, ocularly in the form of eye-lotion, rectally in the form of suppositories.

The pharmaceutical formulations of the invention may be prepared by conventional methods in the art; the nature of the pharmaceutical composition employed will depend on the desired route of administration. The total daily dose usually ranges from about 0.05-2000 mg.

### Methods of Synthesis

Compounds of general formula I may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (Green T. W. and Wuts P. G. M. (1991) Protecting Groups in Organic Synthesis, John Wiley et Sons). These groups are removed at a convenient stage of the compound synthesis using methods that are readily apparent to those skilled in the art. The selection of process as well as the reaction conditions and order of their execution shall be consistent with the preparation of compounds of formula I.

The compound of formula I may be represented as a mixture of enantiomers, which may be resolved into the individual pure R- or S-enantiomers. If for instance, a particular enantiomer of the compound of formula I is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group such as amino, or an acidic functional group such as carboxyl, this resolution may be conveniently performed by fractional crystallization from various solvents, of the salts of the compounds of formula I with optical active acid or by other methods known in the literature, e.g. chiral column chromatography.

Resolution of the final product, an intermediate or a starting material may be performed by any suitable method known in the art as described by Eliel E. L., Wilen S. H. and Mander L. N. (1984) Stereochemistry of Organic Compounds, Wiley-Interscience.

Many of the heterocyclic compounds of formula I can be prepared using synthetic routes well known in the art (Katrizky A. R. and. Rees C. W. (1984) Comprehensive Heterocyclic Chemistry, Pergamon Press).

The product from the reaction can be isolated and purified employing standard techniques, such as extraction, chromatography, crystallization, distillation, and the like.

The compounds of formula I-A wherein W is a 3-substituted piperidine ring may be prepared according to the synthetic sequences illustrated in the Schemes 1-4.

### Wherein

P is an heterocyclic ring with an N—H function as defined above
Q is aryl or heteroaryl as described above
B represents —C(═O)—(C₀-C₂)alkyl-.

The starting material amidoxime can be prepared by methods known in the art of organic synthesis as set forth in part by the following synthesis Scheme 1.

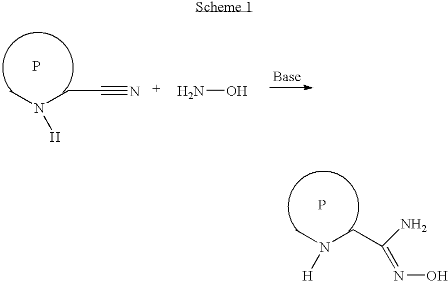

In turn, a nitrile derivative (for example 4-fluoro-benzylnitrile) is reacted with hydroxylamine under neutral or basic conditions such as triethylamine, diisopropyl-ethylamine, sodium carbonate, sodium hydroxide and the like in a suitable solvent (e.g. methyl alcohol, ethyl alcohol). The reaction typically proceeds by allowing the reaction temperature to warm slowly from ambient temperature to a temperature range of 70° C. up to 80° C. inclusive for a time in the range of about 1 hour up to 48 hours inclusive (see for example Lucca, George V. De; Kim, Ui T.; Liang, Jing; Cordova, Beverly; Klabe, Ronald M.; et al; J. Med. Chem.; EN; 41; 13; 1998; 2411-2423, Lila, Christine; Gloanec, Philippe; Cadet, Laurence; Herve, Yolande; Fournier, Jean; et al.; Synth. Commun.; EN; 28; 23; 1998; 4419-4430 and see: Sendzik, Martin; Hui, Hon C.; Tetrahedron Lett.; EN; 44; 2003; 8697-8700 and references therein for reaction under neutral conditions).

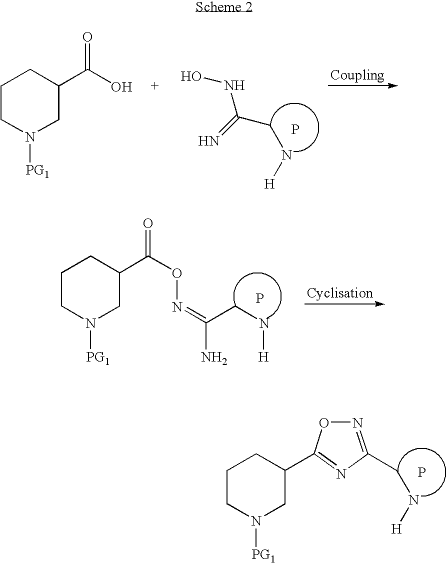

The substituted amidoxime derivative (described in the Scheme 1) may be converted to an acyl-amidoxime derivative using the approach outlined in the Scheme 2. In the Scheme 2, PG₁ is an amino protecting group such as tert-Butyloxycarbonyl, Benzyloxycarbonyl, Ethoxycarbonyl, Benzyl and the like. The coupling reaction may be promoted by coupling agents known in the art of organic synthesis such as EDCI (1-(3-Dimethylaminopropyl)-3-ethylcarbodiimide), DCC (N,N′-Dicyclohexyl-carbodiimide), in the presence of a suitable base such as triethylamine, diisopropyl-ethylamine, in a suitable solvent (e.g. tetrahydrofuran, dichloromethane, N,N-dimethylformamide, dioxane). Typically, a co-catalyst such as HOBT (Hydroxy-benzotriazole), HOAT (1-Hydroxy-7-azabenzotriazole) may also be present in the reaction mixture. The reaction typically proceeds at a temperature in the range of ambient temperature up to 60° C. inclusive for a time in the range of about 2 hours up to 12 hours to produce the intermediate acyl-amidoxime. The cyclisation reaction may be effected thermally in a temperature range of about 80° C. up to about 150° C. for a time in the range of about 2 hours up to 18 hours (see for example Suzuki, Takeshi; Iwaoka, Kiyoshi; Imanishi, Naoki; Nagakura, Yukinori; Miyata, Keiji; et al.; Chem. Pharm. Bull.; EN; 47; 1; 1999; 120-122). The product from the reaction can be isolated and purified employing standard techniques, such as extraction, chromatography, crystallization, distillation, and the like.

The final step may be effected either by a process described in the Scheme 3 or by a process described in the Scheme 4.

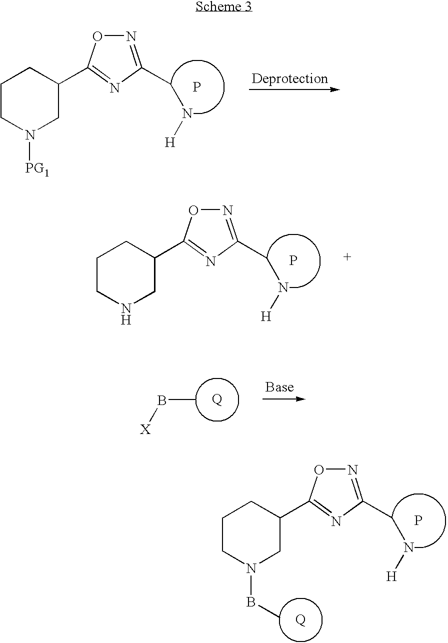

As shown in the Scheme 3, protecting groups PG₁ are removed using standard methods. In the Scheme 3, B is as defined above, X is halogen, for example the piperidine derivative is reacted with an aryl or heteroaryl acyl chloride using method that are readily apparent to those skilled in the art. The reaction may be promoted by a base such as triethylamine, diisopropylamine, pyridine in a suitable solvent (e.g. tetrahydrofuran, dichloromethane). The reaction typically proceeds by allowing the reaction temperature to warm slowly from 0° C. up to ambient temperature for a time in the range of about 4 up to 12 hours.

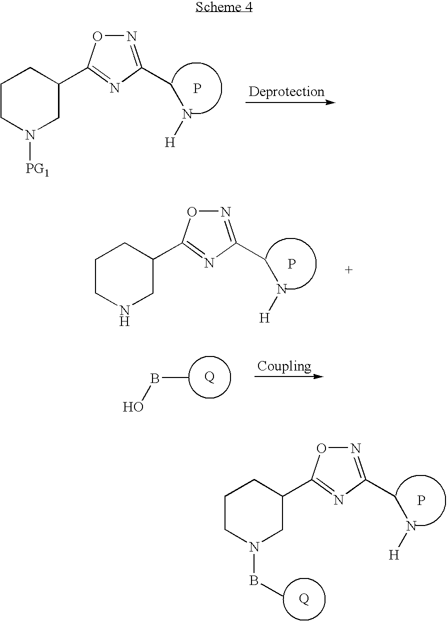

As shown in the Scheme 4, protecting groups PG₁ are removed using standard methods. The coupling reaction may be promoted by coupling agents known in the art of organic synthesis such as EDCI (1-(3-Dimethylaminopropyl)-3-ethylcarbodiimide), DCC (N,N′-Dicyclohexyl-carbodiimide) or by polymer-supported coupling agents such as polymer-supported carbodiimide (PS-DCC, ex Argonaut Technologies), in the presence of a suitable base such as triethylamine, diisopropyl-ethylamine, in a suitable solvent (e.g. tetrahydrofuran, dichloromethane, N,N-dimethylformamide, dioxane). Typically, a co-catalyst such as HOBT (1-Hydroxy-benzotriazole), HOAT (1-Hydroxy-7-azabenzotriazole) and the like may also be present in the reaction mixture. The reaction typically proceeds at ambient temperature for a time in the range of about 2 hours up to 12 hours.

The compounds of formula II-B wherein J is a CH2 and R1, R2 are H may be prepared according to the synthetic sequences illustrated in the Schemes 5.

### Wherein

P is a heterocyclic ring with an N—H function as defined above
Q is aryl or heteroaryl as described above
B represents —C(═O)—(C₀-C₂)alkyl-.

The oxadiazole ring described below is prepared following synthetic routes well known in the art (Katrizky A. R. and Rees C. W. (1984) Comprehensive Heterocyclic Chemistry, Pergamon Press).

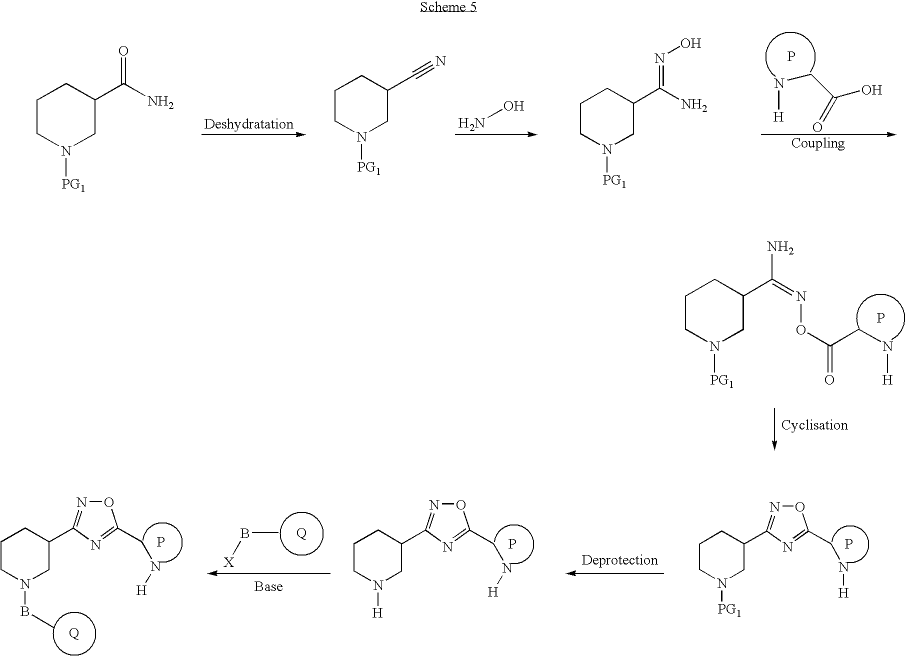

The starting nitrile derivative is reacted with hydroxylamine under neutral or basic conditions such as triethylamine, diisopropyl-ethylamine, sodium carbonate, sodium hydroxide and the like in a suitable solvent (e.g. methyl alcohol, ethyl alcohol). The reaction typically proceeds by allowing the reaction temperature to warm slowly from ambient temperature to a temperature range of 70° C. up to 80° C. inclusive for a time in the range of about 1 hour up to 48 hours inclusive (see for example Lucca, George V. De; Kim, Ui T.; Liang, Jing; Cordova, Beverly; Klabe, Ronald M.; et al; J. Med. Chem.; EN; 41; 13; 1998; 2411-2423, Lila, Christine; Gloanec, Philippe; Cadet, Laurence; Herve, Yolande; Fournier, Jean; et al.; Synth. Commun.; EN; 28; 23; 1998; 4419-4430 and see: Sendzik, Martin; Hui, Hon C.; Tetrahedron Lett.; EN; 44; 2003; 8697-8700 and references therein for reaction under neutral conditions).

The substituted amidoxime derivative (described in the Scheme 5) may be converted to an acyl-amidoxime derivative using the approach outlined in the Scheme 1. In the Scheme 1, PG₁ is an amino protecting group such as tert-Butyloxycarbonyl, Benzyloxycarbonyl, Ethoxycarbonyl, Benzyl and the like. The coupling reaction may be promoted by coupling agents known in the art of organic synthesis such as EDCI (1-(3-Dimethylaminopropyl)-3-ethylcarbodiimide), DCC (N,N′-Dicyclohexyl-carbodiimide), in the presence of a suitable base such as triethylamine, diisopropyl-ethylamine, in a suitable solvent (e.g. tetrahydrofuran, dichloromethane, N,N-dimethylformamide, dioxane). Typically, a co-catalyst such as HOBT (Hydroxy-benzotriazole), HOAT (1-Hydroxy-7-azabenzotriazole) may also be present in the reaction mixture. The reaction typically proceeds at a temperature in the range of ambient temperature up to 60° C. inclusive for a time in the range of about 2 hours up to 12 hours to produce the intermediate acyl-amidoxime. The cyclisation reaction may be performed thermically by warming the reaction mixture without the purification of the acyl-amidoxime intermediate in a temperature range of about 80° C. up to about 150° C. for a time in the range of about 2 hours up to 18 hours (see for example Suzuki, Takeshi; Iwaoka, Kiyoshi; Imanishi, Naoki; Nagakura, Yukinori; Miyata, Keiji; et al.; Chem. Pharm. Bull.; EN; 47; 1; 1999; 120-122). Otherwise the acyl-amidoxime can be isolated and purified employing standard techniques and then cyclised. The cyclization reaction is typically carried out under basic condition such as triethylamine, diisopropyl-ethylamine, sodium carbonate, sodium hydroxide and the like in a suitable solvent (e.g. acetonitrile, dioxane). The reaction typically proceeds in temperature range of about 80° C. up to about 150° C. for a time in the range of about 2 hours up to 18 hours.

The product from the reaction can be isolated and purified employing standard techniques, such as extraction, chromatography, crystallization, distillation, and the like.

Then, the protecting group PG₁ is removed using standard methods. In the Scheme 5, B is as defined above, X is halogen or hydroxyl; for example the piperidine derivative is reacted with an aryl or heteroaryl acyl chloride using method that are readily apparent to those skilled in the art. The reaction may be promoted by a base such as triethylamine, diisopropylamine, pyridine in a suitable solvent (e.g. tetrahydrofuran, dichloromethane). The reaction typically proceeds by allowing the reaction temperature to warm slowly from 0° C. up to ambient temperature for a time in the range of about 4 up to 12 hours.

When X is OH, the coupling reaction may be promoted by coupling agents known in the art of organic synthesis such as EDCI (1-(3-dimethylaminopropyl)-3-ethylcarbodiimide), DCC (N,N′-dicyclohexyl-carbodiimide) or by polymer-supported coupling agents such as polymer-supported carbodiimide (PS-DCC, ex Argonaut Technologies), in the presence of a suitable base such as triethylamine, diisopropyl-ethylamine, in a suitable solvent (e.g. tetrahydrofuran, dichloromethane, N,N-dimethylformamide, dioxane). Typically, a co-catalyst such as HOBT (1-hydroxy-benzotriazole), HOAT (1-hydroxy-7-azabenzotriazole) and the like may also be present in the reaction mixture. The reaction typically proceeds at ambient temperature for a time in the range of about 2 hours up to 12 hours.

The compounds of Formula I which are basic in nature can form a wide variety of different pharmaceutically acceptable salts with various inorganic and organic acids. These salts are readily prepared by treating the base compounds with a substantially equivalent amount of the chosen mineral or organic acid in a suitable organic solvent such as methanol, ethanol or isopropanol (see Stahl P. H., Wermuth C. G., Handbook of Pharmaceuticals Salts, Properties, Selection and Use, Wiley, 2002).

The following non-limiting examples are intending to illustrate the invention. The physical data given for the compounds exemplified is consistent with the assigned structure of those compounds.

### EXAMPLES

Unless otherwise noted, all starting materials were obtained from commercial suppliers and used without further purification.

Specifically, the following abbreviation may be used in the examples and throughout the specification.

g (grams)
mg (milligrams)
mL (millilitres)
μl (microliters)
M (molar)
MHz (megahertz)
mmol (millimoles)
Min (minutes)
AcOEt (ethyl acetate)
K₂CO₃ (potassium carbonate)
CDCl₃ (deuteriated chloroform)
EDCI•HCl (1-3(Dimethylaminopropyl)-3-
ethylcarbodiimide, hydrochloride)
EtOH (ethyl alcohol)
% (percent)
DCM (dichloromethane)
DIEA (diisopropyl ethyl amine)
Mp (melting point)
rt (room temperature)
MeOH (methanol)
Hz (Hertz)
LCMS (Liquid Chromatography Mass Spectrum)
HPLC (High Pressure Liquid Chromatography)
NMR (Nuclear Magnetic Resonance)
1H (proton)
Na₂SO₄ (sodium sulphate)
MgSO₄ (magnesium sulphate)
HOBT (1-hydroxybenzotriazole)
RT (Retention Time)
NaOH (sodium hydroxide)
h (hour)
HCl (hydrochloric acid)
n-BuLi (n-butyllithium)
THF (tetrahydrofuran)

All references to brine refer to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Brucker 500 MHz or on a Brucker 300 MHz. Chemical shifts are expressed in parts of million (ppm, δ units). Coupling constants are in units of hertz (Hz) Splitting patterns describe apparent multiplicities and are designated as s (singlet), d (doublet), t (triplet), q (quadruplet), quint (quintuplet), m (multiplet).

LCMS were recorded under the following conditions:

Method A) Waters Alliance 2795 HT Micromass ZQ. Column Waters XTerra MS C18 (50×4.6 mm, 2.5 μm). Flow rate 1 ml/min Mobile phase: A phase=water/CH₃CN 95/5+0.05% TFA, B phase=water/CH₃CN=5/95+0.05% TFA. 0-1 min (A: 95%, B: 5%), 1-4 min (A: 0%, B: 100%), 4-6 min (A: 0%, B: 100%), 6-6.1 min (A: 95%, B: 5%). T=35° C.; UV detection: Waters Photodiode array 996, 200-400 nm.
Method B) Waters Alliance 2795 HT Micromass ZQ. Column Waters XTerra MS C18 (50×4.6 mm, 2.5 μm). Flow rate 1.2 mL/min Mobile phase: A phase=water/CH₃CN 95/5+0.05% TFA, B phase=water/CH₃CN=5/95+0.05% TFA. 0-0.8 min (A: 95%, B: 5%), 0.8-3.3 min (A: 0%, B: 100%), 3.3-5 min (A: 0%, B: 100%), 5-5.1 min (A: 95%, B: 5%). T=35° C.; UV detection: Waters Photodiode array 996, 200-400 nm.
Method C): Pump 515, 2777 Sample Manager, Micromass ZQ Single quadrupole (Waters). Column 2.1×50 mm stainless steel packed with 3.5 μm SunFire RP C-18 (Waters); flow rate 0.25 mL/min splitting ratio MS:waste/1:4; mobile phase: A phase=water/acetonitrile 95/5+0.1% TFA, B phase=water/acetonitrile 5/95+0.1% TFA. 0-1.0 min (A: 98%, B: 2%), 1.0-5.0 min (A: 0%, B: 100%), 5.0-9.0 min (A: 0%, B: 100%), 9.1-12 min (A: 98%, B: 2%); UV detection wavelength 254 nm; Injection volume: 5 μl
Method D) Waters Alliance 2795 HT Micromass ZQ. Column Waters Symmetry C18 (75×4.6 mm, 3.5 μm). Flow rate 1.5 ml/min. Mobile phase: A phase=water/CH₃CN 95/5+0.05% TFA, B phase=water/CH₃CN=5/95+0.05% TFA. 0-0.5 min (A: 95%, B: 5%), 0.5-7 min (A: 0%, B: 100%), 7-8 min (A: 0%, B: 100%), 8-8.1 min (A: 95%, B: 5%). T=35° C.; UV detection: Waters Photodiode array 996, 200-400 nm.
Method E) Waters Alliance 2795 HT Micromass ZQ. Column Waters Symmetry C18 (75×4.6 mm, 3.5 μm). Flow rate 1.5 ml/min. Mobile phase: A phase=water/CH₃CN 95/5+0.05% TFA, B phase=water/CH₃CN=5/95+0.05% TFA. 0-0.1 min (A: 95%, B: 5%), 6 min (A: 0%, B: 100%), 6-8 min (A: 0%, B: 100%), 8.1 min (A: 95%, B: 5%). T=35° C.; UV detection: Waters Photodiode array 996, 200-400 nm.
Method F) Waters Alliance 2795 HT Micromass ZQ. Column Waters Symmetry C18 (75×4.6 mm, 3.5 μm). Flow rate 1.0 ml/min. Mobile phase: A phase=water/CH₃CN 95/5+0.05% TFA, B phase=water/CH₃CN=5/95+0.05% TFA. 0-1 min (A: 95%, B: 5%), 11 min (A: 0%, B: 100%), 11-12 min (A: 0%, B: 100%), 12.1 min (A: 95%, B: 5%). T=35° C.; UV detection: Waters Photodiode array 996, 200-400 nm.
Method G) Waters Alliance 2795 HT Micromass ZQ. Column Waters Atlantis C18 (75×4.6 mm, 3.0 μm). Flow rate 1.5 ml/min. Mobile phase: A phase=water/CH₃CN 95/5+0.05% TFA, B phase=water/CH₃CN=5/95+0.05% TFA. 0-0.5 min (A: 95%, B: 5%), 5.5 min (A: 0%, B: 100%), 5.5-8 min (A: 0%, B: 100%), 8.1 min (A: 95%, B: 5%). T=35° C.; UV detection: Waters Photodiode array 996, 200-400 nm.
Method H): UPLC system Waters Acquity, Micromass ZQ2000 Single quadrupole (Waters). Column 2.1*50 mm stainless steel packed with 1.7 μm Acquity UPLC-BEH; flow rate 0.50 ml/min; mobile phase: A phase=water/acetonitrile 95/5+0.05% TFA, B phase=water/acetonitrile 5/95+0.05% TFA. 0-0.1 min (A: 95%, B: 5%), 1.6 min (A: 0%, B: 100%), 1.6-1.9 min (A: 0%, B: 100%), 2.4 min (A: 95%, B: 5%); UV detection wavelength 254 nm.
Method I): UPLC system Waters Acquity, Micromass ZQ2000 Single quadrupole (Waters). Column 2.1*50 mm stainless steel packed with 1.7 μm Acquity UPLC-BEH; flow rate 0.50 ml/min; mobile phase: A phase=water/acetonitrile 95/5+0.05% TFA, B phase=water/acetonitrile 5/95+0.05% TFA. 0-0.3 min (A: 95%, B: 5%), 3.3 min (A: 0%, B: 100%), 3.3-3.9 min (A: 0%, B: 100%), 4.4 nm in (A: 95%, B: 5%); UV detection wavelength 254 nm.
Method L): UPLC system Waters Acquity, Micromass ZQ2000 Single quadrupole (Waters). Column 2.1*50 mm stainless steel packed with 1.7 μm Acquity UPLC-BEH; flow rate 0.50 ml/min; mobile phase: A phase=water/acetonitrile 95/5+0.05% TFA, B phase=water/acetonitrile 5/95+0.05% TFA. 0-0.1 min (A: 95%, B: 5%), 3.1 min (A: 0%, B: 100%), 3.1-3.9 min (A: 0%, B: 100%), 4.4 min (A: 95%, B: 5%); UV detection wavelength 254 nm.
Method M) Waters Alliance 2795 HT Micromass ZQ. Column Waters Symmetry C18 (75×4.6 mm, 3.5 μm). Flow rate 1.0 ml/min. Mobile phase: A phase=water/CH₃CN 95/5+0.05% TFA, B phase=water/CH₃CN=5/95+0.05% TFA. 0-0.1 min (A: 95%, B: 5%), 9 min (A: 0%, B: 100%), 9-12 min (A: 0%, B: 100%), 12.1 min (A: 95%, B: 5%). T=35° C.; UV detection: Waters Photodiode array 996, 200-400 nm.
Method N): HPLC system: Waters Acquity, MS detector: Waters ZQ2000. Column: Acquity UPLC-BEH C18 50×2.1 mm×1.7 um; flow rate 0.4 ml/min; mobile phase: A phase=water/acetonitrile 95/5+0.1% TFA, B phase=water/acetonitrile 5/95+0.1% TFA. 0-0.25 min (A: 98%, B: 2%), 0.25-4.0 min (A: 0%, B: 100%), 4.0-5.0 min (A: 0%, B: 100%), 5.1-6 min (A: 98%, B: 2%); UV detection wavelength 254 nm.
Method O): HPLC system: Waters Acquity, MS detector: Waters ZQ2000. Column: Acquity UPLC-BEH C18 50×2.1 mm×1.7 um; flow rate 0.6 ml/min; mobile phase: A phase=water/acetonitrile 95/5+0.1% TFA, B phase=water/acetonitrile 5/95+0.1% TFA. 0-0.25 min (A: 98%, B: 2%), 3.30 min (A: 0%, B: 100%), 3.3-4.0 min (A: 0%, B: 100%), 4.1 min (A: 98%, B: 2%); UV detection wavelength 254 mm.
Method P): HPLC system: Waters Acquity, MS detector: Waters ZQ2000. Column: Acquity UPLC-BEH C18 50×2.1 mm×1.7 um; flow rate 0.3 ml/min; mobile phase: A phase=water/acetonitrile 95/5+0.1% TFA, B phase=water/acetonitrile 5/95+0.1% TFA. 0-0.5 min (A: 98%, B: 2%), 2.0 min (A: 20%, B: 80%), 6.0 min (A: 0%, B: 100%), 6.0-9.5 min (A: 0%, B: 100%), 9.6 min (A: 98%, B: 2%), 9.6-11.0 min (A: 98%, B: 2%); UV detection wavelength 254 nm.
Method Q): Pump 1525u (Waters), 2777 Sample Manager, Micromass ZQ2000 Single quadrupole (Waters); PDA detector: 2996 (Waters). Column 2.1*30 mm stainless steel packed with 3.0 μm Luna C18; flow rate 0.25 ml/min splitting ratio MS:waste/1:4; mobile phase: A phase=water/acetonitrile 95/5+0.1% TFA, B phase=water/acetonitrile 5/95+0.1% TFA. 0-11.0 min (A: 98%, B: 2%), 1.0-5.0 min (A: 0%, B: 100%), 5.0-9.0 min (A: 0%, B: 100%), 9.1-12 min (A: 98%, B: 2%); UV detection wavelength 254 nm; Injection volume: 5 μl.
Method R): Pump 1525u (Waters), 2777 Sample Manager, Micromass ZQ2000 Single quadrupole (Waters); PDA detector: 2996 (Waters). Column Fusion RP-C18, 20×2 mm×2 um; flow rate 0.25 ml/min splitting ratio MS:waste/1:4; mobile phase: A phase=water/acetonitrile 95/5+0.1% TFA, B phase=water/acetonitrile 5/95+0.1% TFA. 0-1.0 min (A: 98%, B: 2%), 1.0-5.0 min (A: 0%, B: 100%), 5.0-9.0 min (A: 0%, B: 100%), 9.1-12 min (A: 98%, B: 2%); UV detection wavelength 254 nm; Injection volume: 5 μl.
Method S): Pump 1525u (Waters), 2777 Sample Manager, Micromass ZQ2000 Single quadrupole (Waters); PDA detector: 2996 (Waters). Column: Acquity UPLC-BEH C18 50×2.1 mm×1.7 um; flow rate 0.25 ml/min splitting ratio MS:waste/1:4; mobile phase: A phase=water/acetonitrile 95/5+0.1% TFA, B phase=water/acetonitrile 5/95+0.1% TFA. 0-11.0 min (A: 98%, B: 2%), 1.0-5.0 min (A: 0%, B: 100%), 5.0-9.0 min (A: 0%, B: 100%), 9.1-12 min (A: 98%, B: 2%); UV detection wavelength 254 nm; Injection volume: 5 μl.
Method T): Pump 1525u (Waters), 2777 Sample Manager, Micromass ZQ2000 Single quadrupole (Waters); PDA detector: 2996 (Waters). Column: Ascentis 100×2.1 mm×3 um; flow rate 0.3 ml/min; mobile phase: A phase=water/acetonitrile 95/5+0.1% TFA, B phase=water/acetonitrile 5/95+0.1% TFA. 0-0.5 min (A: 98%, B: 2%), 2.0 min (A: 20%, B: 80%), 6.0 min (A: 0%, B: 100%), 6.0-9.5 min (A: 0%, B: 100%), 9.6 min (A: 98%, B: 2%), 9.6-11.0 min (A: 98%, B: 2%); UV detection wavelength 254 nm.

All mass spectra were taken under electrospray ionisation (ESI) methods.

Most of the reaction were monitored by thin-layer chromatography on 0.25 mm Macherey-Nagel silica gel plates (60F-2254), visualized with UV light. Flash column chromatography was performed on silica gel (220-440 mesh, Fluka).

Melting point determination was performed on a Buchi B-540 apparatus.

The microwave oven used is an apparatus from Biotage (Optimizer™) equipped with an internal probe that monitors reaction temperature and pressure, and maintains the desired temperature by computer control.

### Example 1

### (4-Fluoro-phenyl)-{(S)-3-[3-(1H-pyrrol-2-yl)-[1,2,4]oxadiazol-5-yl]-piperidin-1-yl}-methanone

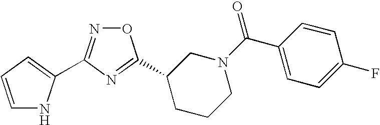

### 1(A) (S)-3-[3-(1H-Pyrrol-2-yl)-[1,2,4]oxadiazol-5-yl]-piperidine-1-carboxylic Acid tert-butyl Ester

To a solution of 1H-Pyrrole-2-carbonitrile (0.110 mL, 1.3 mmol) in EtOH (2 mL), hydroxylamine (50% wt. aqueous solution, 0.318 mL, 5.2 mmol) was added at room temperature and the solution was stirred under reflux for 2 hours. The solvent was removed under reduced pressure to afford N-Hydroxy-1H-pyrrole-2-carboxamidine that was used immediately for the next step.

A mixture of N-Hydroxy-1H-pyrrole-2-carboxamidine (1.3 mmol), S-1-Boc-piperidine-3-carboxylic acid (0.3 g, 1.3 mmol), EDCI.HCl (0.374 g, 1.95 mmol) and HOBT (0.2 g, 1.3 mmol) in dioxane (6 mL) was stirred for 2 h at room temperature, under nitrogen atmosphere, then the reaction mixture was heated under reflux for 7 h. The solvent was evaporated under reduced pressure. The residue was diluted with water (20 mL) and DCM (20 mL), the phases were separated and the organic layer was washed sequentially with water (20 mL×2 times) and with NaOH 1N (20 mL×2 times). The organic layer was dried over Na₂SO₄ and concentrated under reduced pressure. Purification of the crude by flash chromatography (silica gel, eluent: DCM/MeOH/99/1/) gave 0.11 g of (S)-3-[3-(1H-Pyrrol-2-yl)-[1,2,4]oxadiazol-5-yl]-piperidine-1-carboxylic acid tert-butyl ester.

Yield: 26%; (brown oil); LCMS (RT): 5.45 min (Method A); MS (ES+) gave m/z: 318.2 (MH+).

### 1(B) (S)-3-[3-(1H-Pyrrol-2-yl)-[1,2,4]oxadiazol-5-yl]-piperidine Hydrochloride

(S)-3-[3-(1H-Pyrrol-2-yl)-[1,2,4]oxadiazol-5-yl]-piperidine-1-carboxylic acid tert-butyl ester (0.11 g, 0.35 mmol) was dissolved in dioxane (2 mL) and 2 mL of HCl 4N (dioxane solution) were added dropwise at 0° C. The resulting mixture was stirred at room temperature for 1 h. The solvent was evaporated under reduced pressure to afford 76 mg (yield: quantitative) of (S)-3-[3-(1H-Pyrrol-2-yl)-[1,2,4]oxadiazol-5-yl]-piperidine hydrochloride as a white solid.

Yield: quantitative; (brown solid); LCMS (RT): 0.65 min (Method A); MS (ES+) gave m/z: 218.2 (MH+).

### 1(C) (4-Fluoro-phenyl)-{(S)-3-[3-(1H-pyrrol-2-yl)-[1,2,4]oxadiazol-5-yl]-piperidin-1-yl}-methanone

To a suspension of (S)-3-[3-(1H-Pyrrol-2-yl)-[1,2,4]oxadiazol-5-yl]-piperidine hydrochloride (76 mg, 0.35 mmol) in dry dichloromethane (15 mL), triethylamine (0.12 mL, 0.87 mmol) and 4-fluorobenzoyl chloride (0.045 mL, 0.38 mmol) were added dropwise at 0° C. The reaction mixture was allowed to warm at room temperature and stirred under nitrogen atmosphere overnight. The solution was then treated with NaOH 1N (10 mL) and the phases were separated. The organic layer was washed with water (5 mL) and with brine (5 mL), then was dried over Na₂SO₄ and evaporated under reduced pressure. The crude was purified by flash chromatography (silica gel, eluent: DCM/MeOH/NH₄OH 98:2:0.2) to give 80 mg of the title compound.

Yield: 58% (white powder); mp=130-135° C.; [α]_{D}²⁰=+118.13 (c=1.02, MeOH); LCMS (RT): 6.63 min (Method O); MS (ES+) gave m/z: 341.2 (MH+).

¹H-NMR (DMSO-d₆), δ (ppm): 11.52 (s br, 1H); 7.47 (dd, 2H); 7.23 (dd, 2H); 6.97 (m, 1H); 6.74 (m, 1H); 6.21 (m, 1H); 4.22 (m, 1H); 3.77 (m, 1H); 3.50 (dd, 1H); 3.35 (ddd, 1H); 3.27 (ddd, 1H); 2.24 (m, 1H); 1.96 (m, 1H); 1.82 (m, 1H); 1.63 (m, 1H).

### Example 2

### (2,4-Difluoro-phenyl)-((S)-3-[3-(1H-pyrrol-2-yl)-[1,2,4]oxadiazol-5-yl]-piperidin-1-yl)-methanone

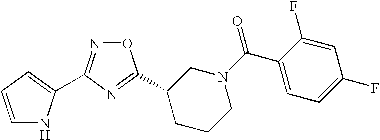

The compound was prepared following the procedure described in the Example 1 (C), starting from (S)-3-[3-(1H-Pyrrol-2-yl)-[1,2,4]oxadiazol-5-yl]-piperidine hydrochloride (prepared as described in the Example 1(B)). The final compound was purified by preparative HPLC.

Yield 20% (brown oil); LCMS (RT): 6.59 min (Method Q); MS (ES+) gave m/z: 359.1 (MH+).

¹H-NMR (DMSO-d₆), δ (ppm): 11.53 (s br, 1H); 7.46 (ddd, 1H); 7.25 (ddd, 1H); 7.14 (ddd, 1H); 6.97 (m, 1H); 6.74 (m, 1H); 6.22 (m, 1H); 4.35 (s br, 1H); 3.91 (s br, 1H); 3.52 (dd, 1H); 3.40-3.18 (m, 2H); 2.24 (m, 1H); 1.97 (m, 1H); 1.82 (m, 1H); 1.62 (m, 1H).

### Example 3

### (3,4-Difluoro-phenyl)-{(S)-3-[3-(1H-pyrrol-2-yl)-[1,2,4]oxadiazol-5-yl]-piperidin-1-yl}-methanone

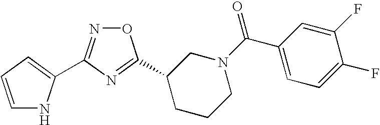

The compound was prepared following the procedure described in the Example 1(C), starting from (S)-3-[3-(1H-Pyrrol-2-yl)-[1,2,4]oxadiazol-5-yl]-piperidine hydrochloride (prepared as described in the Example 1(B)). The final compound was purified by preparative HPLC.

Yield: 25% (brown oil); LCMS (RT): 6.65 min (Method Q); MS (ES+) gave m/z: 359.1 (MH+).

¹H-NMR (DMSO-d₆), δ (ppm): 11.54 (s br, 1H); 7.46 (m, 2H); 7.27 (m, 1H); 6.97 (m, 1H); 6.74 (m, 1H); 6.21 (m, 1H); 4.20 (m, 1H); 3.74 (m, 1H); 3.51 (dd, 1H); 3.41-3.23 (m, 2H); 2.24 (m, 1H); 1.95 (m, 1H); 1.82 (m, 1H); 1.64 (m, 1H).

### Example 4

### (6-Fluoro-pyridin-3-yl)-{(S)-3-[3-(1H-pyrrol-2-yl)-[1,2,4]oxadiazol-5-yl]-piperidin 1-yl}-methanone

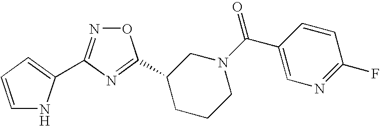

A mixture of (S)-3-[3-(1H-Pyrrol-2-yl)-[1,2,4]oxadiazol-5-yl]-piperidine hydrochloride (0.1 g, 0.39 mmol, prepared as described in the Example 1(B)), 6-Fluoronicotinic acid (66 mg, 0.47 mmol), HOAT (80 mg, 0.59 mmol), PS-DCC (ex Argonaut Technologies, 0.66 g, 0.79 mmol, loading: 1.2 mmol/g) and TEA (0.14 mL, 1 mmol) in dry dichloromethane (10 mL) was kept overnight under orbital shaking (IKA Vibrax VXR). The resin was filtered off and washed repeatedly with dichloromethane; the filtrate was washed with HCl 1N (10 mL×2 times), with NaOH 1N (aq.) (10 mL×2 times) and with brine, then was dried over sodium sulphate and evaporated under reduced pressure. The crude was purified by flash chromatography (silica gel, eluent: AcOEt/Hexane 7/3) to give 28 mg of (6-Fluoro-pyridin-3-yl)-{(S)-3-[3-(1H-pyrrol-2-yl)-[1,2,4]oxadiazol-5-yl]-piperidin-1-yl}-methanone.

Yield: 23% (white solid); mp=131-132° C.; [α]_{D}²⁰=+45.54 (c=0.67, MeOH); LCMS (RT): 7.04 min (Method Q); MS (ES+) gave m/z: 342.2 (MH+).

¹H-NMR (DMSO-d₆), δ (ppm): 11.54 (s br, 1H); 8.32 (m, 1H); 8.03 (ddd, 1H); 7.22 (ddd, 1H); 6.97 (m, 1H); 6.74 (m, 1H); 6.22 (m, 1H); 4.22 (m, 1H); 3.76 (m, 1H); 3.55 (dd, 1H); 3.44-3.28 (m, 2H); 2.24 (m, 1H); 1.98 (m, 1H); 1.81 (m, 1H); 1.67 (m, 1H).

### Example 5

### (3,4-Difluoro-phenyl)-{3-[5-(1H-pyrrol-2-yl)-[1,2,4]oxadiazol-3-yl]-piperidin-1-yl}-methanone

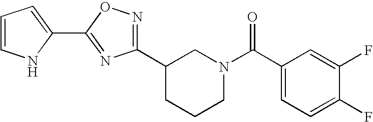

### 5(A) 3-Carbamoyl-piperidine-1-carboxylic Acid Tert-Butyl Ester

Triethylamine (0.96 mL, 6.89 mmol) and then ethyl chloroformate (0.69 mL, 7.23 mmol) were added dropwise at 0° C. to a solution of 1-Boc-piperidine-3-carboxylic acid (1.58 g, 6.89 mmol) in chloroform (10 mL), under nitrogen atmosphere. After stirring 10 min at 0° C., NH₃ (gas) was bubbled into the solution for 1 h. The reaction mixture was then stirred at room temperature for 3 h, 5% NaHCO₃ (aq) was added and the phases were separated. The organic layer was dried over sodium sulphate and evaporated under reduced pressure to afford the title compound, which was used for the next step without further purification.

Yield: quantitative; LCMS (RT): 3.31 min (Method A); MS (ES+) gave m/z: 229.0

### 5(B) 3-Cyano-piperidine-1-carboxylic Acid tert-butyl Ester

Phosphorus oxychloride (0.64 mL, 6.89 mmol) was added dropwise at 0° C. to a solution of 3-carbamoyl-piperidine-1-carboxylic acid tert-butyl ester (1.58 g, 6.89 mmol) in pyridine (15 mL), under nitrogen atmosphere. After stirring overnight at room temperature, ethyl acetate was added and the solution was washed with 10% HCl (2 times). The phases were separated and the organics were dried over sodium sulphate and evaporated to dryness under reduced pressure.

The title compound was used for the next step without further purification. Yield: quantitative; LCMS (RT): 4.48 min (Method A); MS (ES+) gave m/z: 211.1 (MH+).

### 5(C) 3-(N-Hydroxycarbamimidoyl)-piperidine-1-carboxylic Acid tert-butyl Ester

A solution of 3-cyano-piperidine-1-carboxylic acid tert-butyl ester (1.4 g, 6.89 mmol) and aqueous hydroxylamine (50% in water, 1.7 mL, 27.5 mmol) in ethanol (15 mL) was refluxed for 2 h. The solvent was evaporated under reduced pressure to afford the title compound that was used for the next step without further purification.

Yield: quantitative; LCMS (RT): 2.71 min (Method A); MS (ES+) gave m/z: 244.0 (MH+).

### 5(D) 3-[5-(1H-Pyrrol-2-yl)-[1,2,4]oxadiazol-3-yl]-piperidine-1-carboxylic Acid tert-butyl Ester

A mixture of 3-N-hydroxycarbamimidoyl)-piperidine-1-carboxylic acid tert-butyl ester (0.4 g, 1.6 mmol), 1H-pyrrole-2-carboxylic acid (182 mg, 1.6 mmol), HOBT (248 mg, 1.6 mmol), EDCI.HCl (0.47 g, 2.5 mmol) and dry triethylamine (0.461 mL, 3.29 mmol) in dry dioxane (4 mL) was kept under stirring at ambient temperature for 20 h, under nitrogen atmosphere. The reaction mixture was then refluxed for 5 h and the solvent was evaporated under reduced pressure. The residue was diluted with water (15 mL) and ethyl acetate (15 mL), the phases were separated and the organic layer was washed sequentially with water (10 mL, twice), Na₂CO₃ 1N (10 mL, twice) and with brine. The organic layer was dried over sodium sulphate and the solvent was removed under vacuum to give a residue that was purified by flash chromatography (silica gel, eluent: petroleum ether/ethyl acetate 4:1) to give the pure title compound (110 mg).

Yield: 38%; LCMS (RT): 5.54 min (Method A); MS (ES+) gave m/z: 319.1 (MH+).

### 5(E) 3-[5-(1H-Pyrrol-2-yl)-[1,2,4]oxadiazol-3-yl]-piperidine Hydrochloride

To a solution of 3-[5-(1H-Pyrrol-2-yl)-[1,2,4]oxadiazol-3-yl]-piperidine-1-carboxylic acid tert-butyl ester (0.110 g, 0.35 mmol) in dichloromethane (5 mL), 1.5 mL of HCl 4N (dioxane solution) were added at 0° C. and the reaction mixture was allowed to warm at room temperature and stirred for 20 h. The solvent was evaporated under reduced pressure to give the title compound as a white solid, which was used for the next step without further purification.

Yield: quantitative; LCMS (RT): 2.25 min (Method A); MS (ES+) gave m/z: 219.1 (MH+).

### 5(F) (3,4-Difluoro-phenyl)-{3-[5-(1H-pyrrol-2-yl)-[1,2,4]oxadiazol-3-yl]-piperidin-1-yl}-methanone

To a suspension of 3-[5-(1H-Pyrrol-2-yl)-[1,2,4]oxadiazol-3-yl]-piperidinehydrochloride (88 mg, 0.35 mmol) in dry dichloromethane (5 mL), triethylamine (145 μL, 1 mmol) and 3,4-difluorobenzoyl chloride (52 μL, 0.4 mmol) were added dropwise at 0° C. The reaction mixture was allowed to warm at room temperature and stirred for 30 minutes under nitrogen atmosphere. The solution was then treated with water (5 mL) and the phases were separated. The organic layer was washed subsequently with HCl 0.5 N (10 mL, 2 times), 5% NaHCO₃ (10 mL, twice), then was dried over Na₂SO₄ and evaporated under reduced pressure. The crude was purified by flash chromatography (silica gel, eluent petroleum ether:AcOEt 1:1) to afford 49 mg of the title compound.

Yield: 70% (white solid); mp=177° C.; LCMS (RT): 6.88 min (Method Q); MS (ES+) gave m/z: 359.1 (MH+).

H-NMR (DMSO-d₆), δ (ppm): 12.02 (s br, 1H); 7.44 (m, 2H); 7.26 (m, 1H); 7.12 (dd, 1H); 6.96 (dd, 1H); 6.30 (dd, 1H); 4.22 (m, 1H); 3.80 (m, 1H); 3.34 (dd, 1H); 3.22 (ddd, 1H); 3.10 (m, 1H); 2.19 (m, 1H); 1.96-1.76 (m, 2H); 1.64 (m, 1H).

### Example 6

### (2,4-Difluoro-phenyl)-{3-[5-(1H-pyrrol-2-yl)-[1,2,4]oxadiazol-3-yl]-piperidin-1-yl}-methanone

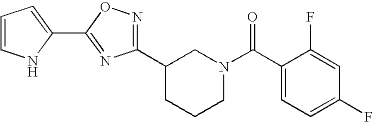

The compound was prepared following the procedure described in the Example 5(F), starting from 3-[5-(1H-pyrrol-2-yl)-[1,2,4]oxadiazol-3-yl]-piperidine hydrochloride (prepared as described in the Example 5(E)). Purification of the final compound was performed by flash chromatography on silica gel (eluent: Hexane:AcOEt 1:1)

Yield: 61% (white solid); mp=151° C.; LCMS (RT): 7.11 min (Method Q); MS (ES+) gave m/z: 359.1.

¹H-NMR (DMSO-d₆), δ (ppm): 12.02 (s br, 1H); 7.45 (m, 1H); 7.22 (m, 1H); 7.12 (m, 2H); 6.96 (d, 1H); 6.30 (dd, 1H); 4.57 (m br, 1H); 3.95 (m br, 1H); 3.44-3.13 (m, 2H); 3.05 (m, 1H); 2.19 (m, 1H); 1.96-1.74 (m, 2H); 1.59 (m, 1H).

### Example 7

### (4-Fluoro-phenyl)-{3-[5-(1H-pyrrol-2-yl)-[1,2,4]oxadiazol-3-yl]-piperidin-1-yl}-methanone

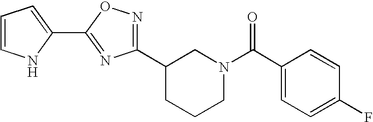

The compound was prepared following the procedure described in the Example 5(F), starting from 3-[5-(1H-pyrrol-2-yl)-[1,2,4]oxadiazol-3-yl]-piperidine hydrochloride (prepared as described in the Example 5(E)). Purification of the final compound was performed by flash chromatography on silica gel (eluent: Hexane:AcOEt 1:1)

Yield: 52% (white solid); mp=158° C.; LCMS (RT): 6.88 min (Method Q); MS (ES+) gave m/z: 341.2 (MH+).

¹H-NMR (DMSO-d₆), δ (ppm): 12.03 (s br, 1H); 7.47 (dd, 2H); 7.22 (dd, 2H); 7.12 (dd, 1H); 6.96 (dd, 1H); 6.30 (dd, 1H); 4.26 (m, 1H); 3.83 (m, 1H); 3.32 (dd, 1H); 3.19 (ddd, 1H); 3.08 (m, 1H); 2.19 (m, 1H); 1.96-1.76 (m, 2H); 1.63 (m, 1H).

### Example 8

### (6-Fluoro-pyridin-3-yl)-{3-[5-(1H-pyrrol-2-yl)-[1,2,4]oxadiazol-3-yl]-piperidin-1-yl}-methanone

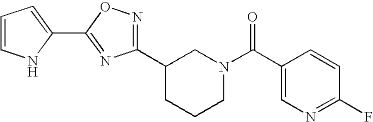

A mixture of 3-[5-(1H-pyrrol-2-yl)-[1,2,4]oxadiazol-3-yl]-piperidine hydrochloride (50 mg, 0.2 mmol; prepared as described in the Example 5(E)), 6-Fluoro-nicotinic acid (32 mg, 0.23 mmol), EDCI.HCl (56 mg, 0.3 mmol), HOBT (44 mg, 0.3 mmol) and TEA (0.083 mL, 0.59 mmol) in DCM (3 mL) was stirred overnight at room temperature, under nitrogen atmosphere. The solvent was evaporated under reduced pressure. The residue was diluted with water (5 mL) and ethyl acetate (10 mL), the phases were separated and the organic layer was washed with Na₂CO₃ 2N (5 mL×2 times) and dried over Na₂SO₄. Evaporation of the solvent under reduced pressure gave a crude solid that was purified by flash chromatography on silica gel eluent petroleum ether/ethyl acetate 1:1).

Yield: 56% (white solid); mp=143° C.; LCMS (RT): 6.44 min (Method Q); MS (ES+) gave m/z: 342.1 (MH+).

¹H-NMR (DMSO-d₆), δ (ppm): 12.03 (s br, 1H); 8.31 (m, 1H); 8.02 (ddd, 1H); 7.21 (ddd, 1H); 7.13 (dd, 1H); 6.96 (dd, 1H); 6.30 (dd, 1H); 4.24 (m, 1H); 3.81 (m, 1H); 3.46-3.21 (m, 2H); 3.13 (m, 1H); 2.19 (m, 1H); 1.97-1.76 (m, 2H); 1.65 (m, 1H).

### Example 9

### (4-Fluoro-2-methyl-phenyl)-{3-[5-(1H-pyrrol-2-yl)-[1,2,4]oxadiazol-3-yl]-piperidin-1-yl}-methanone

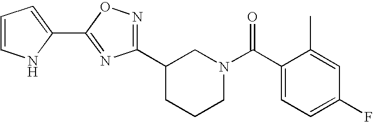

The compound was prepared following the procedure described in the Example 8, using 4-fluoro-2-methyl-benzoic acid as acid of choice and starting from 3-[5-(1H-pyrrol-2-yl)-[1,2,4]oxadiazol-3-yl]-piperidine hydrochloride (prepared as described in the Example 5(E)). Purification of the final compound was performed by flash chromatography on silica gel (eluent petroleum ether/ethyl acetate 1:1)

Yield: 43% (white solid); mp=203° C.; LCMS (RT): 6.68 min (Method Q); MS (ES+) gave m/z: 355.2 (MH+).

¹H-NMR (DMSO-d₆), δ (ppm): 12.02 (s br, 1H); 7.22 (m, 1H); 7.15-6.92 (m, 4H); 6.30 (dd, 1H); 4.56 (m br, 1H); 3.79 (m br, 1H); 3.32 (dd, 1H); 3.21-2.99 (m, 2H); 2.24 (s, 3H); 2.19 (m, 1H); 1.96-1.72 (m, 2H); 1.58 (m, 1H).

### Example 10

### (3,4-Difluorophenyl)-{3-[5-(1H-pyrrol-2-yl)-[1,2,4]oxadiazol-3-yl]-piperidin-1-yl}-methanone

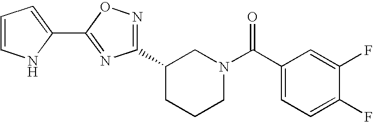

### 10(A) (S)-3-Carbamoyl-piperidine-1-carboxylic Acid tert-butyl Ester

Triethylamine (1.21 mL, 8.72 mmol) and then ethyl chloroformate (0.8 mL, 8.30 mmol) were added dropwise at 0° C. to a solution of (S)-1-Boc-piperidine-3-carboxylic acid (2 g, 8.72 mmol) in chloroform (40 mL), under nitrogen atmosphere. After stirring 10 min at 0° C., NH₃ (gas) was bubbled into the solution for 1 h. The reaction mixture was then stirred at room temperature for 3 h, 5% NaHCO₃ (aq) was added and the phases were separated. The organic layer was dried over sodium sulphate and evaporated under reduced pressure to afford the title compound, which was used for the next step without further purification.

Yield: quantitative; LCMS (RT): 3.31 min (Method A); MS (ES+) gave m/z: 229.0 (MH+).

### 10(B) (S)-3-Cyano-piperidine-1-carboxylic Acid tert-butyl Ester

Phosphorus oxychloride (812 μL, 8.72 mmol) was added dropwise at 0° C. to a solution of (S)-3-carbamoyl-piperidine-1-carboxylic acid tert-butyl ester (2 g, 8.72 mmol) in pyridine (20 mL), under nitrogen atmosphere. After stirring overnight at room temperature, ethyl acetate was added and the solution was washed with 10% HCl (2 times). The phases were separated and the organics were dried over sodium sulphate and evaporated to dryness under reduced pressure. The title compound was used for the next step without further purification.

Yield: quantitative; LCMS (RT): 4.48 min (Method A); MS (ES+) gave m/z: 211.1 (MH+).

### 10(C) (S)-3-(N-Hydroxycarbamimidoyl)-piperidine-1-carboxylic Acid tert-butyl Ester

A solution of (S)-3-cyano-piperidine-1-carboxylic acid tert-butyl ester (1.8 g, 8.72 mmol) and aqueous hydroxylamine (50% in water, 2.1 mL, 34.88 mmol) in ethanol (20 mL) was refluxed for 2 h. The solvent was evaporated under reduced pressure to afford the title compound that was used for the next step without further purification.

Yield: quantitative; LCMS (RT): 2.71 min (Method A); MS (ES+) gave m/z: 244.0 (MH+).

### 10(D) (S)-3-[5-(1H-Pyrrol-2-yl)-[1,2,4]oxadiazol-3-yl]-piperidine-1-carboxylic Acid tert-butyl Ester

A mixture of (S)-3-(N-hydroxycarbamimidoyl)-piperidine-1-carboxylic acid tert-butyl ester (0.4 g, 1.6 mmol), prepared as described in Example 10(C), 1H-pyrrole-2-carboxylic acid (182 mg, 1.6 mmol), HOBT (248 mg, 1.6 mmol), EDCI.HCl (0.47 g, 2.5 mmol) and dry triethylamine (0.461 mL, 3.29 mmol) in dry dioxane (4 mL) was kept under stirring at ambient temperature for 20 h, under nitrogen atmosphere. The reaction mixture was then refluxed for 5 h and the solvent was evaporated under reduced pressure. The residue was diluted with water (15 mL) and ethyl acetate (15 mL), the phases were separated and the organic layer was washed sequentially with water (10 mL, twice), 1N Na₂CO₃ (10 mL, twice) and with brine. The organic layer was dried over sodium sulphate and the solvent was removed under vacuum to give a residue that was purified by flash chromatography (silica gel, eluent: petroleum ether/ethyl acetate 4:1) to give the pure title compound (110 mg).

Yield: 35%; LCMS (RT): 5.55 min (Method A); MS (ES+) gave m/z: 319.1 (MH+).

### 10E) (S)-3-[5-(1H-Pyrrol-2-yl)-[1,2,4]oxadiazol-3-yl]-piperidine Hydrochloride

To a solution of (S)-3-[5-(1H-pyrrol-2-yl)-[1,2,4]oxadiazol-3-yl]-piperidine-1-carboxylic acid tert-butyl ester (0.110 g, 0.35 mmol) in dichloromethane (5 mL), 1.5 mL of 4N HCl (dioxane solution) were added at 0° C. and the reaction mixture was allowed to warm at room temperature and stirred for 20 h. The solvent was evaporated under reduced pressure to give the title compound as a white solid, which was used for the next step without further purification.

Yield: quantitative; LCMS (RT): 2.25 min (Method A); MS (ES+) gave m/z: 219.1 (MH+).

### 10(F) (3,4-Difluoro-phenyl)-{(S)-3-[5-(1H-pyrrol-2-yl)-[1,2,4]oxadiazol-3-yl]-piperidin-1-yl}-methanone

To a suspension of (S)-3-[5-(1H-pyrrol-2-yl)-[1,2,4]oxadiazol-3-yl]-piperidine hydrochloride (88 mg, 0.35 mmol) in dry dichloromethane (5 mL), triethylamine (145 μL, 1 mmol) and 3,4-difluorobenzoyl chloride (52 μL, 0.4 mmol) were added dropwise at 0° C. The reaction mixture was allowed to warm at room temperature and stirred for 30 minutes under nitrogen atmosphere. The solution was then treated with water (5 mL) and the phases were separated. The organic layer was washed subsequently with 0.5 N HCl (10 mL, 2 times), 5% NaHCO₃ (10 mL, twice), then was dried over Na₂SO₄ and evaporated under reduced pressure. The crude was purified by flash chromatography (silica gel, eluent petroleum ether:AcOEt 1′:1) to afford 49 mg of the title compound.

Yield: 48% (white solid); mp=168° C.; LCMS (RT): 6.42 min (Method Q); MS (ES+) gave m/z: 359.2 (MH+).

H-NMR (DMSO-d₆), δ (ppm): 12.02 (s br, 1H); 7.50-7.38 (m, 2H); 7.27 (m, 1H); 7.12 (dd, 1H); 6.96 (dd, 1H); 6.30 (dd, 1H); 4.22 (m, 1H); 3.80 (m, 1H); 3.34 (dd, 1H); 3.22 (ddd, 1H); 3.10 (ddd, 1H); 2.19 (m, 1H); 1.97-1.76 (m, 2H); 1.63 (m, 1H).

### Example 11

### (4-Fluoro-phenyl)-{3-[5-(1H-indol-2-yl)-[1,2,4]oxadiazol-3-yl]-piperidin-1-yl}-methanone

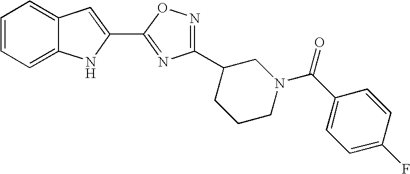

### 11(A) 3-[5-(1H-Indol-2-yl)-[1,2,4]oxadiazol-3-yl]-piperidine-1-carboxylic Acid tert-butyl Ester

A mixture of 3-(N-hydroxycarbamimidoyl)-piperidine-1-carboxylic acid tert-butyl ester (0.3 g, 1.2 mmol, prepared as described in Example 5(C)), 1H-indole-2-carboxylic acid (0.2 g, 1.2 mmol), HOBT (0.17 g, 1.2 mmol), EDCI.HCl (0.71 g, 3.7 mmol) and dry DIEA (0.631 mL, 3.7 mmol) in dry acetonitrile (10 mL) was warmed at 130° C. for 30 minutes in a microwave oven. The solvent was evaporated under reduced pressure and then the residue was diluted with water (15 mL) and ethyl acetate (15 mL), the phases were separated and the organic layer was washed sequentially with water (10 mL, twice), 1N Na₂CO₃ (10 mL, twice) and with brine. The organic layer was dried over sodium sulphate and the solvent was removed under vacuum to give a residue that was purified by flash chromatography (silica gel, eluent: petroleum ether:ethyl acetate 4:1) to give the pure title compound (120 mg).

Yield: 27%; LCMS (RT): 6.47 min (Method A); MS (ES+) gave m/z: 369.1 (MH+).

### 11(B) 2-(3-Piperidin-3-yl-[1,2,4]oxadiazol-5-yl)-1H-indole Hydrochloride

The compound was prepared following the procedure described in the Example 10(E) starting from 3-[5-(1H-indol-2-yl)-[1,2,4]oxadiazol-3-yl]-piperidine-1-carboxylic acid tert-butyl ester (prepared as described in Example 11 (A)).

Yield: quantitative (white powder); LCMS (RT): 3.06 min (Method A); MS (ES+) gave m/z: 269.1 (MH+).

### 11(C) (4-Fluoro-phenyl)-{3-[5-(1H-indol-2-yl)-[1,2,4]oxadiazol-3-yl]-piperidin-1-yl}-methanone

The compound was prepared following the procedure described in the Example 10(F), using 2-(3-piperidin-3-yl-[1,2,4]oxadiazol-5-yl)-1H-indole hydrochloride (prepared as described in the Example 11(B)). Purification of the final compound was performed by flash chromatography on silica gel (eluent: Hexane:AcOEt 6:4)

Yield: 64% (white solid); mp=199-201° C.; LCMS (RT): 7.28 min (Method Q); MS (ES+) gave m/z: 391.2 (MH+).

¹H-NMR (DMSO-d₆), δ (ppm): 12.04 (s br, 1H); 7.70 (dd, 1H); 7.53 (dd, 1H); 7.48 (dd, 2H); 7.34 (dd, 1H); 7.30 (ddd, 1H); 7.23 (dd, 2H); 7.13 (ddd, 1H); 4.31 (m, 1H); 3.85 (m, 1H); 3.38 (dd, 1H); 3.27-3.11 (m, 2H); 2.25 (m, 1H); 2.00-1.78 (m, 2H); 1.65 (m, 1H).

### Example 12

### (2,4-Difluoro-phenyl)-{(S)-3-[3-(1H-indol-2-yl)-[1,2,4]oxadiazol-5-yl]-piperidin-1-yl}-methanone

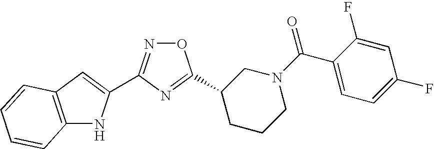

### 12 (A) (S)-3-[3-(1H-Indol-2-yl)-[1,2,4]oxadiazol-5-yl]-piperidine-1-carboxylic Acid tert-butyl Ester

The compound was prepared following the procedure described in the Example 1 (A), starting from 1H-indole-2-carbonitrile. (S)-3-[3-(1H-Indol-2-yl)-[1,2,4]oxadiazol-5-yl]-piperidine-1-carboxylic acid tert-butyl ester was used without further purification.

Yield: quantitative (brown oil); LCMS (RT): 6.41 min (Method A); MS (ES+) gave m/z: 369.1 (MH+).

### 12(B) 2-((S)-5-Piperidin-3-yl-[1,2,4]oxadiazol-3-yl)-1H-indole Hydrochloride

The compound was prepared following the procedure described in the Example 1 (B), starting from (S)-3-[3-(1H-indol-2-yl)-[1,2,4]oxadiazol-5-yl]-piperidine-1-carboxylic acid tert-butyl ester.

Yield: quantitative (brown solid); LCMS (RT): 2.63 min (Method B); MS (ES+) gave m/z: 269.1 (MH+).

### 12 (C) (2,4-Difluoro-phenyl)-f{(S)-3-[3-(1H-indol-2-yl)-[1,2,4]oxadiazol-5-yl]-piperidin-1-yl}-methanone

The compound was prepared following the procedure described in the Example 1 (C), starting from 2-((S)-5-piperidin-3-yl-[1,2,4]oxadiazol-3-yl)-1H-indole hydrochloride. (2,4-difluoro-phenyl)-{(S)-3-[3-(1H-indol-2-yl)-[1,2,4]oxadiazol-5-yl]-piperidin-1-yl}-methanone was obtained pure after flash column chromatography (silica gel, eluent: AcOEt:petroleum ether 3:7).

Yield: 3% (white solid); LCMS (RT): 7.13 min (Method Q); MS (ES+) gave m/z: 409.3 (MH+).

¹H-NMR (DMSO-d₆), δ (ppm): 11.67 (s br, 1H); 7.65 (d, 1H); 7.52-7.43 (m, 2H); 7.30-7.03 (m, 5H); 4.41 (m, 1H); 3.98 (m, 1H); 3.58 (dd, 1H); 3.45-3.19 (m, 2H); 2.29 (m, 1H); 2.02 (m, 1H); 1.84 (m, 1H); 1.65 (m, 1H).

### Example 13

### (4-Fluoro-phenyl)-{3-[5-(2H-pyrazol-3-yl)-[1,2,4]oxadiazol-3-yl]-piperidin-1-yl}-methanone

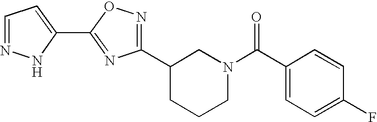

### 13(A) 3-[5-(2H-Pyrazol-3-yl)-[1,2,4]oxadiazol-3-yl]-piperidine-1-carboxylic Acid tert-butyl Ester

A mixture of 3-(N-hydroxycarbamimidoyl)-piperidine-1-carboxylic acid tert-butyl ester (0.5 g, 2.05 mmol, prepared as described in 5 (C)), 2H-pyrazole-3-carboxylic acid (0.23 mg, 2.05 mmol), HOBT (0.31 mg, 2.05 mmol), EDCI.HCl (0.59 g, 3.08 mmol) and dry triethylamine (1.1 mL, 4 mmol) in dry dioxane (8 mL) was kept under stirring at ambient temperature for 5 h, under nitrogen atmosphere. The reaction mixture was then diluted with DCM and washed with 5% NaHCO₃ and brine. The organic layer was dried over Na₂SO₄ and concentrated. The crude was purified on silica gel (eluent: DCM:MeOH 20:1.5) to afford 520 mg of 3-{[(hydroxyimino]-[(2H-pyrazole-3-carbonyl)-amino]-methyl}-piperidine-1-carboxylic acid tert-butyl ester (yield: 75%; LCMS (RT): 3.18 min (Method A); MS (ES+) gave m/z: 338.06).

A solution of 3-{[(hydroxyimino)]-[(2H-pyrazole-3-carbonyl)-amino]-methyl}-piperidine-1-carboxylic acid tert-butyl ester (0.52 g, 1.54 mmol) and triethylamine (0.43 mL, 3.086 mmol) in dioxane (4 mL) was refluxed for 14 h and then the solvent was partially removed under vacuo. The solid precipitated was filtered to afford 360 mg of the title compound

Yield: 73% (white solid); LCMS (RT): 3.5 min (Method A); MS (ES+) gave m/z: 320.1 (MH+).

### 13(B) 3-[5-(2H-Pyrazol-3-yl)-[1,2,4]oxadiazol-3-yl]-piperidine Hydrochloride

The compound was prepared following the procedure described in the Example 5(E) starting from 3-[5-(2H-pyrazol-3-yl)-[1,2,4]oxadiazol-3-yl]-piperidine-1-carboxylic acid tert-butyl ester (prepared as described in Example 13(A))

Yield: quantitative (white powder); LCMS (RT): 1.1 min (Method C); MS (ES+) gave m/z: 220.1 (MH+).

### 13(C) (4-Fluoro-phenyl)-{3-[5-(2H-pyrazol-3-yl)-[1,2,4]oxadiazol-3-yl]-piperidin-1-yl}-methanone

The compound was prepared following the procedure described in the Example 5(F), using 3-[5-(2H-pyrazol-3-yl)-[1,2,4]oxadiazol-3-yl]-piperidine hydrochloride (prepared as described in the Example 13(B)). Purification of the final compound was performed by flash chromatography on silica gel (eluent: AcOEt:Hexane 3:1)

Yield: 62% (amorphous white solid); LCMS (T.R.): 6.90 min (Method Q); MS (ES+) gave m/z: 342.2 (MH+).

¹H-NMR (DMSO-d₆), δ (ppm): 13.60 (s br, 1H); 7.93 (d, 1H); 7.47 (dd, 2H); 7.23 (dd, 2H); 6.92 (d, 1H); 4.23 (m, 1H); 3.83 (m, 1H); 3.37 (dd, 1H); 3.27-3.09 (m, 2H); 2.20 (m, 1H); 1.98-1.76 (m, 2H); 1.63 (m, 1H).

### Example 14

### (3,4-Difluoro-phenyl)-{3-[5-(2H-pyrazol-3-yl)-[1,2,4]oxadiazol-3-yl]-piperidin-1-yl}-methanone

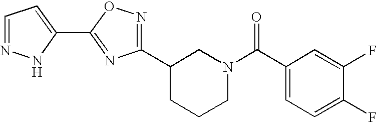

The compound was prepared following the procedure described in the Example 5(F), using 3-[5-(2H-pyrazol-3-yl)-[1,2,4]oxadiazol-3-yl]-piperidine hydrochloride (prepared as described in the Example 13(B)). Purification of the final compound was performed by flash chromatography on silica gel (eluent: AcOEt:petroleum ether 3:1)

Yield: 54% (amorphous white solid); LCMS (RT): 7.07 min (Method Q); MS (ES+) gave m/z: 360.2 (MH+).

¹H-NMR (DMSO-d₆), δ (ppm): 13.61 (s br, 1H); 7.93 (d, 1H); 7.51-7.39 (m, 2H); 7.27 (m, 1H); 6.92 (d, 1H); 4.19 (m, 1H); 3.79 (m, 1H); 3.39 (dd, 1H); 3.30-3.11 (m, 2H); 2.19 (m, 1H); 2.00-1.76 (m, 2H); 1.64 (m, 1H).

### Example 15

### (3,4-Difluoro-phenyl)-{(S)-3-[3-(1H-indol-2-yl)-[1,2,4]oxadiazol-5-yl]-piperidin-1-yl}-methanone

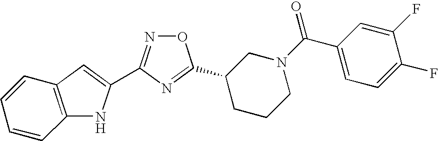

The compound was prepared following the procedure described in the Example 1 (C), starting from 2-((S)-5-piperidin-3-yl-[1,2,4]oxadiazol-3-yl)-1H-indole hydrochloride (prepared as described in Example 12 (B)). (3,4-Difluoro-phenyl)-{(S)-3-[3-(1H-indol-2-yl)-[1,2,4]oxadiazol-5-yl]-piperidin-1-yl}-methanone was obtained pure after flash column chromatography (silica gel, eluent: AcOEt:petroleum ether 3:7).

Yield: 13% (white solid); mp=93-94° C.; LCMS (RT): 7.11 min (Method Q); MS (ES+) gave m/z: 409.2 (MH+).

¹H-NMR (DMSO-d₆), δ (ppm): 11.70 (s br, 1H); 7.64 (d, 1H); 7.53-7.42 (m, 3H); 7.28 (m, 1H); 7.22 (dd, 1H); 7.12 (s br, 1H); 7.07 (dd, 1H); 4.24 (m, 1H); 3.73 (m, 1H); 3.57 (dd, 1H); 3.45 (m, 1H); 3.31 (m, 1H); 2.26 (m, 1H); 2.02 (m, 1H); 1.82 (m, 1H); 1.67 (m, 1H).

### Example 16

### (4-Fluoro-phenyl)-{(S)-3-[3-(1H-indol-2-yl)-[1,2,4]oxadiazol-5-yl]-piperidin-1-yl}-methanone

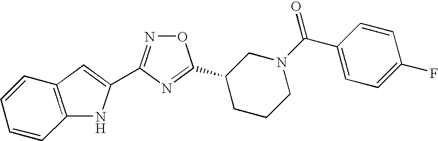

The compound was prepared following the procedure described in the Example 1 (C), starting from 2-((S)-5-piperidin-3-yl-[1,2,4]oxadiazol-3-yl)-1H-indole hydrochloride (prepared as described in Example 12 (B)). (4-Fluoro-phenyl)-{(S)-3-[3-(1H-indol-2-yl)-[1,2,4]oxadiazol-5-yl]-piperidin-1-yl}-methanone was obtained pure after flash column chromatography (silica gel, eluent: AcOEt:petroleum ether 3:7).

Yield: 18% (white solid); LCMS (RT): 6.99 min (Method Q); MS (ES+) gave m/z: 391.2 (MH+).

¹H-NMR (DMSO-d₆), δ (ppm): 11.70 (s, 1H); 7.64 (d, 1H); 7.48 (m, 3H); 7.28-7.18 (m, 3H); 7.12 (m, 1H); 7.07 (dd, 1H); 4.27 (m, 1H); 3.78 (m, 1H); 3.56 (dd, 1H); 3.43 (m, 1H); 3.30 (ddd, 1H); 2.30 (m, 1H); 2.01 (m, 1H); 1.84 (m, 1H); 1.67 (m, 1H).

### Example 17

### (4-Fluoro-phenyl)-{3-[5-(1H-imidazol-2-yl)-[1,2,4]oxadiazol-3-yl]-piperidin-1-yl}-methanone

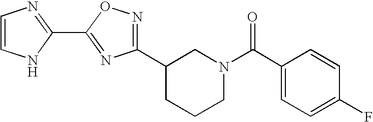

### 17(A) 3-[5-(1H-Imidazol-2-yl)-[1,2,4]oxadiazol-3-yl]-piperidine-1-carboxylic Acid tert-butyl Ester

A mixture of 3-(N-hydroxycarbamidoyl)-piperidine-1-carboxylic acid tert-butyl ester (0.25 g, 1.03 mmol, prepared as described in Example 5 (C)), 1H-imidazole-2-carboxylic acid (116 mg, 1.03 mmol), HOBT (161 mg, 1.05 mmol), EDCI.HCl (0.3 g, 1.55 mmol) and dry triethylamine (0.29 mL, 2.05 mmol) in dry DCM (10 mL) was stirred for 4 h at ambient temperature, under nitrogen atmosphere. The solution was then concentrated under vacuum and the crude was purified on silica gel (eluent: DCM:MeOH 20:1) to afford 100 mg of 3-{[(hydroxyimino]-[(1H-imidazole-2-carbonyl)-amino]-methyl}-piperidine-1-carboxylic acid tert-butyl ester (yield: 29%; LCMS (RT): 2.54 min (Method B); MS (ES+) gave m/z: 357.95 (MH+).).

A solution of 3-{[(hydroxyimino]-[(1H-imidazole-2-carbonyl)-amino]-methyl}-piperidine-1-carboxylic acid tert-butyl ester (0.1 g, 0.3 mmol) and DIEA (0.043 mL, 0.3 mmol) in MeCN (4 mL) was heated for 30 min at 150° in a sealed tube under microwave irradiation. Upon cooling a white solid precipitated which was collected by filtration to afford 43 mg of the title compound.

Yield: 45% (white solid); LCMS (RT): 2.97 min (Method B); MS (ES+) gave m/z: 320.1 (MH+).

### 17(B) 3-[5-(1H-imidazol-2-yl)-[1,2,4]oxadiazol-3-yl]-piperidine Trifluoroacetate

3-[5-(1H-Imidazole-2-yl)-[1,2,4]oxadiazol-3-yl]-piperidine-1-carboxylic acid tert-butyl ester (40 mg, 0.125 mmol), prepared as described in Example 17 (A), was dissolved in DCM (1 mL) and TFA (1 mL) was added. The solution was stirred for 30 min and then the solvent was removed under vacuum to give the title compound as a colourless gum, which was used without further purification.

Yield: quantitative (colourless gum); LCMS (RT): 0.65 min (Method B); MS (ES+) gave m/z: 220.1 (MH+).

### 17(C) (4-Fluoro-phenyl)-{3-[1H-imidazol-2-yl)-[1,2,4]oxadiazol-3-yl]-piperidin-1-yl}-methanone

4-Fluorobenzoyl chloride (16 μL, 0-13 mmol) was added to a stirred solution of 3-[5-(1H-imidazol-2-yl)-[1,2,4]oxadiazol-3-yl]-piperidine trifluoroacetate (prepared as described in example 19 (B)) and triethylamine (35 μL, 0.25 mmol) in dry DCM (2 mL). The solution was stirred under nitrogen atmosphere for 2 h and then concentrated under vacuum. Purification of the crude was performed by flash chromatography on silica gel (eluent: DCM:MeOH 20:1). The title compound was obtained as a white solid (35 mg)

Yield: 81% (amorphous white solid); LCMS (RT): 5.58 min (Method Q); MS (ES+) gave m/z: 342.1 (MH+).

¹H-NMR (DMSO-d₆, 343K), δ (ppm): 8.80 (s br, 1H); 7.47 (dd, 2H); 7.36 (s, 2H); 7.23 (dd, 2H); 4.29 (m, 1H); 3.83 (m, 1H); 3.34 (dd, 1H); 3.25-3.08 (m, 2H); 2.22 (m, 1H); 1.98-1.77 (m, 2H); 1.64 (m, 1H).

### Example 18

### (3,4-Difluoro-phenyl)-{3-[5-(1H-imidazol-2-yl)-[1,2,4]oxadiazol-3-yl]-piperidin-1-yl}-methanone

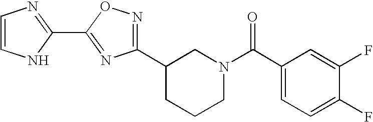

The title compound was obtained following the experimental procedure described in Example 17(C), starting from 3-[5-(1H-imidazol-2-yl)-[1,2,4]oxadiazol-3-yl]-piperidine trifluoroacetate (prepared as described in Example 17(B)) and 3,4-difluorobenzoyl chloride. Purification was performed by trituration from diethyl ether to afford (3,4-difluoro-phenyl)-{3-[5-(1H-imidazol-2-yl)-[1,2,4]oxadiazol-3-yl]-piperidin-1-yl}-methanone as a white solid.

Yield: 60% (white solid); mp=148.5-148.9° C.; LCMS (RT): 6.73 min (Method Q); MS (ES+) gave m/z: 360.2 (MH+).

¹H-NMR (DMSO-d₆, 343K), δ (ppm): 13.51 (s br, 1H); 7.52-7.38 (m, 3H); 7.32-7.20 (m, 2H); 4.21 (m, 1H); 3.79 (m, 1H); 3.45-3.08 (m, 3H); 2.29-2.14 (m, 1H); 2.12-1.46 (m, 3H).

### Example 19

### {(S)-3-[3-(1H-Indol-2-yl)-[1,2,4]oxadiazol-5-yl]-piperidin-1-yl}-(5-methyl-isoxazol-4-yl)-methanone

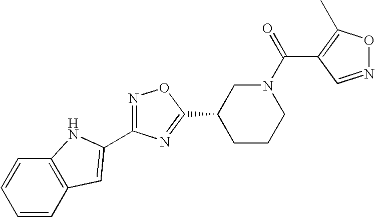

The compound was prepared following the procedure described in the Example 4, starting from 2-((S)-5-Piperidin-3-yl-[1,2,4]oxadiazol-3-yl)-1H-indole hydrochloride (prepared as described in Example 12 (B)) and using 5-Methyl-isoxazole-4-carboxylic acid as the acid of choice. {(S)-3-[3-(1H-Indol-2-yl)-[1,2,4]oxadiazol-5-yl]-piperidin-1-yl}-(5-methyl-isoxazol-4-yl)-methanone was obtained pure after flash column chromatography (silica gel, eluent: DCM).

Yield: 5% (White powder); mp=163-164° C.; LCMS (RT): 6.63 min (Method Q); MS (ES+) gave m/z: 378.2 (MH+).

¹H-NMR (DMSO-d_{6,} 373K), δ (ppm): 11.48 (s br, 1H); 8.54 (s, 1H); 7.64 (d, 1H); 7.51 (d, 1H); 7.22 (dd, 1H); 7.12 (m, 1H); 7.07 (dd, 1H); 4.27 (dd, 1H); 3.80 (ddd, 1H); 3.63 (dd, 1H); 3.48-3.33 (m, 2H); 2.48 (s, 3H); 2.30 (m, 1H); 2.04 (m, 1H); 1.88 (m, 1H); 1.68 (m, 1H).

### Example 20

### (5-Methyl-isoxazol-4-yl)-{(S)-3-[3-(1H-pyrrol-2-yl)-[1,2,4]oxadiazol-5-yl]-piperidin-1-yl}-methanone

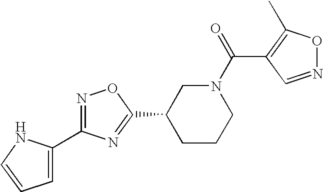

The compound was prepared following the procedure described in the Example 4, starting from (S)-3-[3-(1H-Pyrrol-2-yl)-[1,2,4]oxadiazol-5-yl]-piperidine hydrochloride (prepared as described in Example 1 (B)) and using 5-Methyl-isoxazole-4-carboxylic acid as the acid of choice. (5-Methyl-isoxazol-4-yl)-{(S)-3-[3-(1H-pyrrol-2-yl)-[1,2,4]oxadiazol-5-yl]-piperidin-1-yl}-methanone was obtained pure after flash column chromatography (silica gel, eluent: hexane/ethyl acetate 3:7).

Yield: 60% (White powder); mp=125-127° C.; [α]_{D}²⁰=+47.8 (c=0.68, MeOH); LCMS (RT): 6.01 min (Method Q); MS (ES+) gave m/z: 328.1 (MH+).

¹H-NMR (DMSO-d₆, 373K), δ (ppm): 11.32 (s br, 1H); 8.52 (s, 1H); 6.97 (m, 1H); 6.74 (m, 1H); 6.22 (m, 1H); 4.22 (dd, 1H); 3.78 (ddd, 1H); 3.58 (dd, 1H); 3.36 (m, 2H); 2.46 (s, 3H); 2.24 (m, 1H); 2.06-1.79 (m, 2H); 1.66 (m, 1H).

### Example 21

### (6-Fluoro-pyridin-3-yl)-{(S)-3-[5-(1H-pyrrol-2-yl)-[1,2,4]oxadiazol-3-yl]-piperidin-1-yl}-methanone

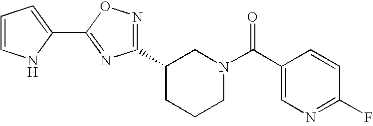

The compound was prepared following the procedure described in the Example 8, starting from ((S)-3-[5-(1H-pyrrol-2-yl)-[1,2,4]oxadiazol-3-yl]-piperidine hydrochloride (prepared as described in Example 10 (E)) and using 6-fluoro-pyridine-3-carboxylic acid as the acid of choice. (6-Fluoro-pyridin-3-yl)-{(S)-3-[5-(1H-pyrrol-2-yl)-[1,2,4]oxadiazol-3-yl]-piperidin-1-yl}-methanone was obtained pure after flash column chromatography (silica gel, eluent: petroleum ether/ethyl acetate 1:1).

Yield: 49% (white solid); mp=147° C.; [α]_{D}²⁰=+118.45 (c=1.005, MeOH); LCMS (RT): 6.03 min (Method Q); MS (ES+) gave m/z: 342.1 (MH+).

¹H-NMR (DMSO-d_{6,} 343K), δ (ppm): 12.05 (s br, 1H); 8.32 (m, 1H); 8.03 (ddd, 1H); 7.21 (ddd, 1H); 7.13 (dd, 1H); 6.96 (dd, 1H); 6.30 (dd, 1H); 4.23 (m, 1H); 3.81 (m, 1H); 3.37 (dd, 1H); 3.26 (ddd, 1H); 3.13 (m, 1H); 2.19 (m, 1H); 1.97-1.76 (m, 2H); 1.66 (m, 1H).

### Example 22

### (4-Fluoro-phenyl)-{(S)-3-[5-(1H-pyrrol-2-yl)-[1,2,4]oxadiazol-3-yl]-piperidin-1-yl}-methanone

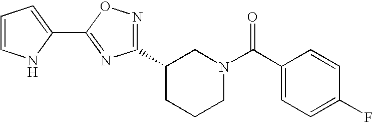

The compound was prepared following the procedure described in the Example 1(C), starting from ((S)-3-[5-(1H-pyrrol-2-yl)-[1,2,4]oxadiazol-3-yl]-piperidine hydrochloride (prepared as described in Example 10 (E)) and using 4-fluorobenzoyl chloride as the acylating agent. (4-Fluoro-phenyl)-{(S)-3-[5-(1H-pyrrol-2-yl)-[1,2,4]oxadiazol-3-yl]-piperidin-1-yl}-methanone was obtained pure after flash column chromatography (silica gel, eluent: petroleum ether/ethyl acetate 1:1).

Yield: 54% (white solid); mp=181° C.; [α]_{D}²⁰=+108.05 (c=0.975, MeOH); LCMS (RT): 6.41 min (Method Q); MS (ES+) gave m/z: 341.2 (MH+).

¹H-NMR (DMSO-d₆, 343K), δ (ppm): 12.04 (s br, 1H); 7.47 (dd, 2H); 7.22 (dd, 2H); 7.12 (m, 1H); 6.96 (m, 1H); 6.30 (dd, 1H); 4.26 (m, 1H); 3.83 (m, 1H); 3.31 (dd, 1H); 3.19 (ddd, 1H); 3.08 (m, 1H); 2.19 (m, 1H); 1.95-1.76 (m, 2H); 1.62 (m, 1H).

### Example 23

### (6-Fluoro-pyridin-3-yl)-{3-[5-(1H-indol-2-yl)-[1,2,4]oxadiazol-3-yl]-piperidin-1-yl}-methanone

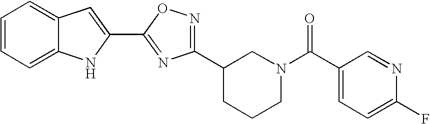

The compound was prepared following the procedure described in the Example 8, starting from 2-(3-piperidin-3-yl-[1,2,4]oxadiazol-5-yl)-1H-indole hydrochloride (prepared as described in Example 11 (B)) and using 6-fluoro-pyridine-3-carboxylic acid as the acid of choice.

Yield: 51% (white solid); mp=163.1-164.3° C.; LCMS (RT): 7.52 min (Method Q); MS (ES+) gave m/z: 392.2 (MH+).

¹H-NMR (DMSO-d_{6,} 343K), δ (ppm): 12.07 (s br, 1H); 8.33 (m, 1H); 8.05 (ddd, 1H); 7.70 (d, 1H); 7.53 (dd, 1H); 7.34 (d, 1H); 7.30 (ddd, 1H); 7.22 (dd, 1H); 7.12 (dd, 1H); 4.28 (m, 1H); 3.83 (m, 1H); 3.43 (dd, 1H); 3.34-3.16 (m, 2H); 2.24 (m, 1H); 1.94 (m, 1H); 1.85 (m, 1H); 1.70 (m, 1H).

### Example 24

### (4-Fluoro-phenyl)-{(S)-3-[3-(1H-imidazol-2-yl)-[1,2,4]oxadiazol-5-yl]-piperidin-1-yl}-methanone

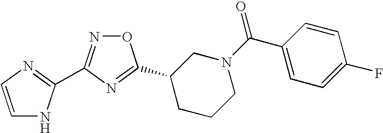

### 24(A) 1H-Imidazole-2-carboxylic Acid Amide

A solution of 1H-imidazole-2-carboxylic acid (200 mg, 1.78 mmol) and thionyl chloride (3 mL) was refluxed for 2 h. The reaction mixture was cooled at room temperature and poured into toluene (5 mL), the resulting precipitate was collected by filtration and then washed with diethyl ether. The solid was dissolved in conc. NH₄OH (aq) (3 mL) and stirred at 10° C. for 1 h, then the mixture was allowed to warm at RT. A solid precipitated out and was filtered, washed with water and dried in a vacuum oven at 40° C. for 1 night to afford 72 mg of 1H-imidazole-2-carboxylic acid amide.

Yield: 36%; LCMS (RT): 0.62 min (Method D); MS (ES+) gave m/z: 112.0 (MH+).

### 24(B) N-Hydroxy-1H-imidazole-2-carboxamidine

A solution of 1H-imidazole-2-carboxylic acid amide (360 mg, 3.24 mmol) and phenyl dichlorophosphate (2 mL) was heated at 170° C. for 8 min, in a microwaves oven. The reaction mixture was cooled at room temperature and poured into water (50 mL). The solution was cooled at 0° C. and the pH was adjusted to 11 by addition of NaOH 10 M. Ethyl acetate was added and the phases were separated. The organic layer was dried over sodium sulphate and evaporated in vacuo to provide 1H-Imidazole-2-carbonitrile. A solution of 1H-imidazole-2-carbonitrile and hydroxylamine (50% sol. in water, 794 μL, 13 mmol) in ethanol (15 mL) was refluxed for 4 h. The solvent was removed and the crude N-hydroxy-1H-imidazole-2-carboxamidine was used for the next step without further purification.

Yield: quantitative; LCMS (RT): 0.62 min (Method D); MS (ES+) gave m/z: 127.0 (MH+).

### 24(C) (S)-3-[3-(1H-Imidazol-2-yl)-[1,2,4]oxadiazol-5-yl]-piperidine-1-carboxylic Acid tert-butyl Ester

A mixture of N-hydroxy-1H-imidazole-2-carboxamidine (3.24 mmol), S-1-Boc-piperidine-3-carboxylic acid (0.743 g, 3.24 mmol), EDCI.HCl (0.932 g, 4.86 mmol) and HOBT (0.438 g, 3.24 mmol) in DCM (10 mL) was stirred overnight at room temperature, under nitrogen atmosphere. The mixture was washed with NaHCO₃ (aq), the phases were separated and the organic layer was dried over Na₂SO₄ and concentrated under reduced pressure. Purification of the crude by flash chromatography (silica gel, eluent: DCM/MeOH 98/2) gave a solid that was dissolved in CH₃CN (5 mL), triethylamine (450 μL, 3.24 mmol) was added and the resulting solution was heated at 150° C. for 1 h, in a microwaves oven. The solvent was removed and the crude was purified by flash chromatography (silica gel, eluent: DCM/MeOH 98/2) to give (S)-3-[3-(1H-imidazol-2-yl)-[1,2,4]oxadiazol-5-yl]-piperidine-1-carboxylic acid tert-butyl ester (50 mg).

Yield: 5%; LCMS (RT): 3.21 min (Method D); MS (ES+) gave m/z: 342.11 (MH+).

### 24(D) (S)-3-[3-(1H-Imidazol-2-yl)-[1,2,4]oxadiazol-5-yl]-piperidine Hydrochloride

To a solution of (S)-3-[3-(1H-imidazol-2-yl)-[1,2,4]oxadiazol-5-yl]-piperidine-1-carboxylic acid tert-butyl ester (50 mg, 0.157 mmol) in dichloromethane (1 mL), 1 mL of HCl 4N (dioxane solution) was added at 0° C. and the reaction mixture was allowed to warm at room temperature and stirred for 2 h. The solvent was evaporated under reduced pressure to give the title compound as a white solid, which was used for the next step without further purification.

Yield: quantitative.

### 24(E) (4-Fluoro-phenyl)-{(S)-3-[3-(1H-imidazol-2-yl)-[1,2,4]oxadiazol-5-yl]-piperidin-1-yl}-methanone

The title compound was obtained following the experimental procedure described in Example 1(C), starting from (S)-3-[3-(1H-imidazol-2-yl)-[1,2,4]oxadiazol-5-yl]-piperidine hydrochloride and using 4-fluorobenzoyl chloride as the acylating agent. Purification by preparative HPLC gave (4-fluoro-phenyl)-{(S)-3-[3-(1H-imidazol-2-yl)-[1,2,4]oxadiazol-5-yl]-piperidin-1-yl}-methanone as a colourless oil.

Yield: 12% (colourless oil); LCMS (RT): 5.34 min (Method Q); MS (ES+) gave m/z: 342.2 (MH+).

¹H-NMR (DMSO-d₆ 343K), δ (ppm): 7.48 (dd, 2H); 7.30 (s, 2H); 7.24 (dd, 2H); 4.27 (m, 1H); 3.79 (m, 1H); 3.51 (dd, 1H); 3.42 (ddd, 1H); 3.26 (ddd, 1H); 2.27 (m, 1H); 2.05-1.78 (m, 2H); 1.66 (m, 1H).

### Example 25

### (3,4-Difluoro-phenyl)-{(S)-3-[3-(1H-imidazol-2-yl)-[1,2,4]oxadiazol-5-yl]-piperidin-1-yl}-methanone

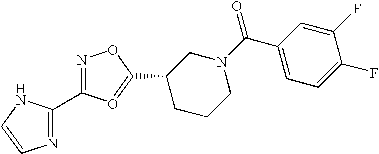

The title compound was obtained following the same experimental procedure described in Example 4, starting from (S)-3-[3-(1H-imidazol-2-yl)-[1,2,4]oxadiazol-5-yl]-piperidine hydrochloride (prepared as described in Example 24 (D)) and using 3,4-difluorobenzoic acid as the acid of choice.

Purification was performed by flash chromatography (silica gel, eluent: DCM/MeOH 98:2).

Yield: 19% (White powder); mp=156-157° C.; [α]_{D}²⁰=+90.0 (c=0.50, MeOH).

LCMS (RT): 5.31 min (Method Q); MS (ES+) gave m/z: 360.2 (MH+).

¹H-NMR (DMSO-d_{6,} 343K), δ (ppm): 12.91 (s br, 1H); 7.53-7.40 (m, 2H); 7.34-7.13 (m, 3H); 4.23 (m, 1H); 3.76 (m, 1H); 3.53 (dd, 1H); 3.43 (ddd, 1H); 3.29 (ddd, 1H); 2.29 (m, 1H); 1.98 (m, 1H); 1.83 (m, 1H); 1.66 (m, 1H).

### Example 26

### {3-[5-(1H-Indol-2-yl)-[1,2,4]oxadiazol-3-yl]-piperidin-1-yl}-(5-methyl-isoxazol-4-yl)-methanone

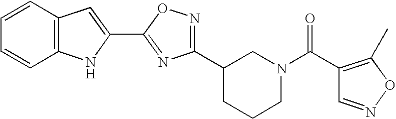

The compound was prepared following the procedure described in the Example 8, starting from 2-(3-piperidin-3-yl-[1,2,4]oxadiazol-5-yl)-1H-indole hydrochloride (prepared as described in Example 11 (B)) and using 5-methyl-isoxazole-4-carboxylic acid as the acid of choice.

Yield: 97% (white solid); mp=175.6-177.2° C.; LCMS (RT): 8.01 min (Method Q); MS (ES+) gave m/z: 378.2 (MH+).

¹H-NMR (DMSO-d_{6,} 343K), δ (ppm): 12.08 (s br, 1H); 8.60 (s, 1H); 7.70 (d, 1H); 7.53 (dd, 1H); 7.35 (dd, 1H); 7.30 (ddd, 1H); 7.13 (ddd, 1H); 4.31 (m, 1H); 3.87 (m, 1H); 3.42 (dd, 1H); 3.28 (ddd, 1H); 3.17 (m, 1H); 2.48 (d, 3H); 2.23 (m, 1H); 2.03-1.79 (m, 2H); 1.66 (m, 1H).

### Example 27

### (4-Fluoro-phenyl)-{(S)-3-[5-(4-methyl-1H-pyrrol-2-yl)-[1,2,4]oxadiazol-3-yl]-piperidin-1-yl}-methanone

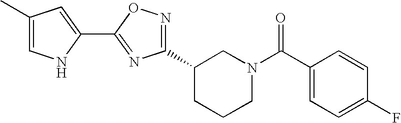

### 27 (A) (S)-3-Carbamoyl-piperidine-1-carboxylic Acid tert-butyl Ester

Triethylamine (1.21 mL, 8.72 mmol) and then ethyl chloroformate (0.8 mL, 8.30 mmol) were added dropwise at 0° C. to a solution of (S)-1-Boc-piperidine-3-carboxylic acid (2 g, 8.72 mmol) in chloroform (40 mL), under nitrogen atmosphere. After stirring 10 min at 0° C., NH₃ (gas) was bubbled into the solution for 1 h. The reaction mixture was then stirred at room temperature for 3 h, 5% NaHCO₃ (aq) was added and the phases were separated. The organic layer was dried over sodium sulphate and evaporated under reduced pressure to afford the title compound, which was used for the next step without further purification.

Yield: quantitative; LCMS (RT): 3.31 min (Method A); MS (ES+) gave m/z: 229.0 (MH+).

### 27 (B) (S)-3-Cyano-piperidine-1-carboxylic Acid tert-butyl Ester

Phosphorus oxychloride (812 μL, 8.72 mmol) was added dropwise at 0° C. to a solution of (S)-3-carbamoyl-piperidine-1-carboxylic acid tert-butyl ester (2 g, 8.72 mmol) in pyridine (20 mL), under nitrogen atmosphere. After stirring overnight at room temperature, ethyl acetate was added and the solution was washed with 10% HCl (2 times). The phases were separated and the organics were dried over sodium sulphate and evaporated to dryness under reduced pressure.

The title compound was used for the next step without further purification.

Yield: quantitative; LCMS (RT): 4.48 min (Method A); MS (ES+) gave m/z: 211.1 (MH+).

### 27 (C) (S)-1-(4-Fluoro-benzoyl)-piperidine-3-carbonitrile

(S)-3-Cyano-piperidine-1-carboxylic acid tert-butyl ester (1.5 g, 7.14 mmol), was dissolved in dioxane (15 mL) and 10 mL of 4N HCl (dioxane solution) were added dropwise at 0° C. The resulting mixture was stirred at room temperature for 5 h. The solvent was evaporated under reduced pressure to afford (S)-piperidine-3-carbonitrile hydrochloride as a white solid, that was used for the next step without further purification.

To a suspension of (S)-piperidine-3-carbonitrile hydrochloride (7.14 mmol) in dry dichloromethane (100 mL), triethylamine (3 mL, 21.4 mmol) and 4-fluorobenzoyl chloride (930 μL, 7.85 mmol) were added dropwise at 0° C. The reaction mixture was allowed to warm at room temperature and stirred for 3 h under nitrogen atmosphere. The solution was then treated with 5% NaHCO₃ (50 mL, twice) and the phases were separated. The organic layer was washed with 1N HCl (50 mL) and with brine (50 mL), then was dried over Na₂SO₄ and evaporated under reduced pressure. The crude was purified by flash chromatography (silica gel, eluent gradient: from petroleum ether/ethyl acetate 7:3 to petroleum ether/ethyl acetate 1:1) to give 1.01 g of the title compound.

Yield: 61% (yellow oil); LCMS (T): 3.7 min (Method D); MS (ES+) gave m/z: 233.1 (MH+).

### 27 (D) (S)-1-(4-Fluoro-benzoyl)-N-hydroxy-piperidine-3-carboxamidine

A solution of (S)-1-(4-fluoro-benzoyl)-piperidine-3-carbonitrile (1.01 g, 4.35 mmol) and aqueous hydroxylamine (50% in water, 1.1 mL, 17.4 mmol) in ethanol (10 mL) was refluxed for 4 h. The solvent was evaporated under reduced pressure to afford the title compound (1.15 g) that was used for the next step without further purification.

Yield: quantitative; ¹H-NMR (DMSO-d₆, 343K), δ (ppm): 8.61 (s br, 1H); 7.44 (dd, 2H); 7.22 (dd, 2H); 5.12 (s br, 2H); 4.00 (m, 2H); 3.17-2.82 (m, 3H); 2.23 (m, 1H); 1.98 (m, 1H); 1.78-1.55 (m, 2H).

### 27 (E) (4-Fluoro-phenyl)-{(S)-3-[5-(4-methyl-1H-pyrrol-2-yl)-[1,2,4]oxadiazol-3-yl]-piperidin-1-yl}-methanone

A mixture of (S)-1-(4-fluoro-benzoyl)-N-hydroxy-piperidine-3-carboxamidine (150 mg, 0.56 mmol), 4-methyl-pyrrole-2-carboxylic acid (70 mg, 0.56 mmol), EDCI.HCl (162 mg, 0.85 mmol) and HOBT (85 mg, 0.56 mmol) in dioxane (2 mL) was stirred at 40° C. for 2 h, then at 90° C. for 20 h, then under reflux for 24 h, under nitrogen atmosphere. The mixture was diluted with ethyl acetate and washed with 1N Na₂CO₃ (aq), the phases were separated and the organic layer was dried over Na₂SO₄ and concentrated under reduced pressure. Purification of the crude by flash chromatography (silica gel, eluent: petroleum ether/ethyl acetate 3:2) gave a solid that was triturated from ethyl acetate/diethyl ether 1:1. (4-Fluoro-phenyl)-{(S)-3-[5-(4-methyl-1H-pyrrol-2-yl)-[1,2,4]oxadiazol-3-yl]-piperidin-1-yl}-methanone was obtained (20 mg).

Yield: 10% (White solid); mp=183° C.; LCMS (RT): 6.69 min (Method Q); MS (ES+) gave m/z: 355.1 (MH+).

¹H-NMR (DMSO-d₆, 343K), δ (ppm): 11.61 (s br, 1H); 7.46 (dd, 2H); 7.21 (dd, 2H); 6.89 (m, 1H); 6.76 (m, 1H); 4.24 (m, 1H); 3.84 (m, 1H); 3.31 (dd, 1H); 3.18 (ddd, 1H); 3.05 (m, 1H); 2.18 (m, 1H); 2.09 (s, 3H); 1.95-1.73 (m, 2H); 1.70-1.51 (m, 1H).

### Example 28

### (6-Fluoro-pyridin-3-yl)-{(S)-3-[5-(4-methyl-1H-pyrrol-2-yl)-[1,2,4]oxadiazol-3-yl]-piperidin-1-yl}-methanone

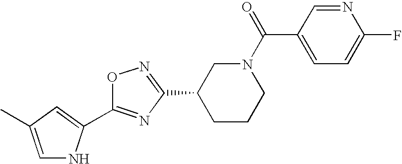

### 28(A) (S)-3-[5-(4-Methyl-1H-pyrrol-2-yl)-[1,2,4]oxadiazol-3-yl]-piperidine-1-carboxylic Acid tert-butyl Ester

A mixture of 4-methyl-pyrrole-2-carboxylic acid (412 mg, 3.28 mmol), HOAT (448 mg, 3.28 mmol), EDCI.HCl (948 mg, 4.92 mmol) in dry dioxane (12 mL) was kept under stirring at 50° C. for 1 h, under nitrogen atmosphere, then (S)-3-(N-hydroxycarbamimidoyl)-piperidine-1-carboxylic acid tert-butyl ester (0.8 g, 3.28 mmol), prepared as described in Example 10(C), was added and the reaction mixture was stirred at 50° C. for 2 h. The solvent was evaporated under reduced pressure. The residue was diluted with water (15 mL) and ethyl acetate (15 mL), the phases were separated and the organic layer was washed sequentially 5% NaHCO₃ (aq) (10 mL, twice) and with brine. The organic layer was dried over sodium sulphate and the solvent was removed under vacuum to give a residue that was purified by flash chromatography (silica gel, eluent: petroleum ether/ethyl acetate 1:1) to give 950 mg of a solid. The solid was dissolved in acetonitrile (10 mL), activated 4 A molecular sieves were added and the mixture was heated at 120° C. for 2 h in a microwaves oven. Ethyl acetate was added and the molecular sieves were filtered off. The filtrate was evaporated under reduced pressure and the crude was purified by flash chromatography (silica gel, eluent: petroleum ether/ethyl acetate 2:1) to give (S)-3-[5-(4-methyl-1H-pyrrol-2-yl)-[1,2,4]oxadiazol-3-yl]-piperidine-1-carboxylic acid tert-butyl ester (464 mg) as a yellow oil.

Yield: 43% (yellow oil); LCMS (RT): 5.3 min (Method E); MS (ES+) gave m/z: 333.2 (MH+).

### 28(B) (S)-3-[5-(4-Methyl-1H-pyrrol-2-yl)-[1,2,4]oxadiazol-3-yl]-piperidine Hydrochloride

To a solution of (S)-3-[5-(4-methyl-1H-pyrrol-2-yl)-[1,2,4]oxadiazol-3-yl]-piperidine-1-carboxylic acid tert-butyl ester (0.46 g, 1.38 mmol) in dichloromethane (20 mL), 3.45 mL of HCl 4N (dioxane solution) were added at 0° C. and the reaction mixture was allowed to warm at room temperature and stirred for 3 h. The solvent was evaporated under reduced pressure to give the title compound as a brown solid, which was used for the next step without further purification.

Yield: quantitative.

### 28(C) (6-Fluoro-pyridin-3-yl)-{(S)-3-[5-(4-methyl-1H-pyrrol-2-yl)-[1,2,4]oxadiazol-3-yl]-piperidin-1-yl}-methanone

A mixture of 6-fluoro-nicotinic acid (63 mg, 0.44 mmol), HOAT (76 mg, 0.55 mmol), EDCI.HCl (107 mg, 0.55 mmol) and triethylamine (156 μL, 1.11 mmol) in dry DCM (10 mL) was kept under stirring at RT for 15 min, under nitrogen atmosphere, then (S)-3-[5-(4-methyl-1H-pyrrol-2-yl)-[1,2,4]oxadiazol-3-yl]-piperidine hydrochloride (0.1 g, 0.37 mmol), was added and the reaction mixture was stirred at RT for 2 h. The mixture was diluted with DCM and was washed sequentially with 5% NaHCO₃ (aq) (10 mL, twice) and with brine. The organic layer was dried over sodium sulphate and the solvent was removed under vacuum to give a residue that was purified by flash chromatography (silica gel, eluent: petroleum ether/ethyl acetate 1.5:1) to give 59 mg of a solid. The solid was then crystallised from EtOH/iPrOH to give 44 mg of (6-fluoro-pyridin-3-yl)-{(S)-3-[5-(4-methyl-1H-pyrrol-2-yl)-[1,2,4]oxadiazol-3-yl]-piperidin-1-yl}-methanone.

Yield: 33% (White solid); [α]_{D}²⁰=+124.5° (c=0.90, MeOH); LCMS (RT): 2.61 min (Method N); MS (ES+) gave m/z: 356.4 (MH+).

¹H-NMR (DMSO-d₆, 353K), δ (ppm): 11.70 (s br, 1H); 8.31 (m, 1H); 8.02 (ddd, 1H); 7.20 (dd, 1H); 6.90 (m, 1H); 6.77 (m, 1H); 4.23 (m, 1H); 3.81 (m, 1H); 3.37 (dd, 1H); 3.26 (ddd, 1H); 3.12 (m, 1H); 2.18 (m, 1H); 2.09 (s, 3H); 1.96-1.76 (m, 2H); 1.65 (m, 1H).

### Example 29

### (5-Methyl-isoxazol-4-yl)-{(S)-3-[5-(4-methyl-1H-pyrrol-2-yl)-[1,2,4]oxadiazol-3-yl]-piperidin-1-yl}-methanone

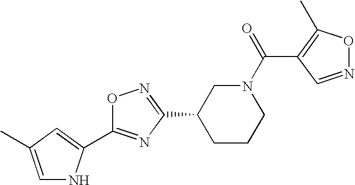

The title compound was prepared following the experimental procedure described in Example 28(C), starting from (S)-3-[5-(4-methyl-1H-pyrrol-2-yl)-[1,2,4]oxadiazol-3-yl]-piperidine hydrochloride, prepared as described in Example 28 (B), and using 5-methyl-isoxazole-4-carboxylic acid as the acid of choice.

Purification was performed by flash chromatography (silica gel, eluent: petroleum ether/ethyl acetate 1.5:1).

Yield: 36% (White solid); [α]_{D}²⁰=+95.0 (c=1.01; MeOH); LCMS (RT): 2.56 min (Method N); MS (ES+) gave m/z: 342.4 (MH+).

¹H-NMR (DMSO-d₆ 353K), δ (ppm): 11.69 (s br, 1H); 8.57 (m, 1H); 6.90 (m, 1H); 6.77 (m, 1H); 4.24 (m, 1H); 3.85 (m, 1H); 3.36 (dd, 1H); 3.26 (ddd, 1H); 3.07 (m, 1H); 2.47 (d, 3H); 2.18 (m, 1H); 2.09 (m, 3H); 1.97-1.77 (m, 2H); 1.63 (m, 1H).

### Example 30

### (2-Fluoro-pyridin-4-yl)-{(S)-3-[5-(4-methyl-1H-pyrrol-2-yl)-[1,2,4]oxadiazol-3-yl]-piperidin-1-yl}-methanone

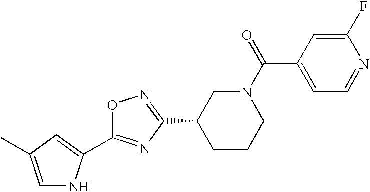

The title compound was prepared following the experimental procedure described in Example 28(C), starting from (S)-3-[5-(4-methyl-1H-pyrrol-2-yl)-[1,2,4]oxadiazol-3-yl]-piperidine hydrochloride, prepared as described in Example 28 (B), and using 2-fluoro-pyridine-4-carboxylic acid as the acid of choice.

Purification was performed by flash chromatography (silica gel, eluent: petroleum ether/ethyl acetate 2:1).

Yield: 49% (White solid); [α]_{D}²⁰=+100.1 (c=0.82, MeOH); LCMS (RT): 2.64 mm (Method N); MS (ES+) gave m/z: 356.4 (MH+).

¹H-NMR (DMSO-d₆, 353K), δ (ppm): 11.69 (s br, 1H); 8.31 (d, 1H); 7.34 (ddd, 1H); 7.16 (m, 1H); 6.90 (m, 1H); 6.77 (m, 1H); 4.60-3.53 (m br, 2H); 3.41-3.07 (m, 3H); 2.18 (m, 1H); 2.10 (s, 3H); 1.96-1.74 (m, 2H); 1.65 (m, 1H).

### Example 31

### (4-Fluoro-phenyl)-{(S)-3-[3-(4-methyl-1H-pyrrol-2-yl)-[1,2,4]oxadiazol-5-yl]-piperidin-1-yl}-methanone

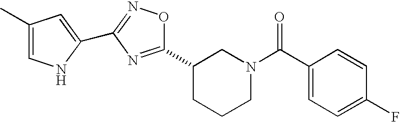

### 31(A) 4-Methyl-1H-pyrrole-2-carboxylic Acid Amide

A solution of 4-methyl-pyrrole-2-carboxylic acid (250 mg, 2 mmol) and carbonyl-diimidazole (356 mg, 2.2 mmol) in acetonitrile (10 mL) was stirred at room temperature for 2 h, then conc. NH₄OH (2 mL) was added and the mixture was heated at 80° C. for 3 h. The solvent was removed, the residue was dissolved in water and treated with 1N HCl to adjust the pH to 1. Ethyl acetate was then added, the phases were separated and the organic layer was dried over magnesium sulphate and evaporated under vacuum. The crude residue was purified by flash chromatography (silica gel cartridge, eluent gradient: from hexane/ethyl acetate 100:0 to hexane/ethyl acetate 0:100) to give 215 mg.

Yield: 87%; LCMS (RT): 2.01 min (Method D); MS (ES+) gave m/z: 125.1 (MH+).

### 31(B) 4-Methyl-1H-pyrrole-2-carbonitrile

A solution of 4-methyl-1H-pyrrole-2-carboxylic acid amide (210 mg, 1.7 mmol) in phosphorus oxychloride (5 mL) was heated at 100° C. for 5 minutes, then the mixture was cooled, ice was added and conc. NH₄OH was added to adjust the pH to 10. Extraction with ethyl acetate was performed, the organic layer was dried over magnesium sulphate and evaporated under vacuum. The crude residue was purified by flash chromatography (silica gel cartridge, eluent gradient: from hexane/ethyl acetate 100:0 to hexane/ethyl acetate 60:40) to give 180 mg.

Yield: 100%; LCMS (RT): 2.74 min (Method B); MS (ES+) gave m/z: 107.0 (MH+).

### 31(C)N-Hydroxy-4-methyl-1H-pyrrole-2-carboxamidine

A solution of 4-methyl-1H-pyrrole-2-carbonitrile (180 mg, 1.7 mmol) and aqueous hydroxylamine (50% in water, 460 μL, 7 mmol) in ethanol (10 mL) was refluxed for 1 h. The solvent was evaporated under reduced pressure and the crude residue was purified by flash chromatography (silica gel cartridge, eluent gradient: from hexane/ethyl acetate 100:0 to hexane/ethyl acetate 0:100) to give 240 mg.

Yield: 100%; LCMS (RT): 0.63 min (Method B); MS (ES+) gave m/z: 140.1 (MH+).

### 31(D) (S)-3-[3-(4-Methyl-1H-pyrrol-2-yl)-[1,2,4]oxadiazol-5-yl]-piperidine-1-carboxylic Acid tert-butyl Ester

A mixture of (S)-N-Boc-nipecotic acid (460 mg, 2 mmol), HOAT (272 mg, 2 mmol), EDCI.HCl (480 mg, 2.5 mmol) in dry DCM (10 mL) was kept under stirring at ambient temperature for 10 minutes, under nitrogen atmosphere, then N-hydroxy-4-methyl-1H-pyrrole-2-carboxamidine (240 mg, 1.7 mmol) was added and stirring at RT was maintained overnight. The solvent was removed under vacuum to give a residue that was purified by flash chromatography (silica gel cartridge, eluent gradient: from hexane/ethyl acetate 100:0 to hexane/ethyl acetate 60:40). The solid thus obtained was dissolved in acetonitrile (2 mL) and heated in a sealed tube at 80° C. for 2 h20, in a microwaves oven. Solvent was removed and the crude residue was purified by flash chromatography (silica gel cartridge, eluent gradient: from hexane/ethyl acetate 100:0 to hexane/ethyl acetate 80:20) to give (S)-3-[3-(4-methyl-1H-pyrrol-2-yl)-[1,2,4]oxadiazol-5-yl]-piperidine-1-carboxylic acid tert-butyl ester.

Yield: 12%; LCMS (RT): 5.84 min (Method D); MS (ES+) gave m/z: 333.1 (MH+).

### 31 (E) (S)-3-[3-(4-Methyl-1H-pyrrol-2-yl)-[1,2,4]oxadiazol-5-yl]-piperidine Trifluoroacetate

To a solution of (S)-3-[3-(4-methyl-1H-pyrrol-2-yl)-[1,2,4]oxadiazol-5-yl]-piperidine-1-carboxylic acid tert-butyl ester (50 mg, 0.15 mmol) in dichloromethane (2 mL), 0.5 mL of TFA were added at 0° C. and the reaction mixture was stirred at 0° C. for 1 h, in the dark. The solvent was evaporated under reduced pressure to give the title compound, which was used for the next step without further purification.

Yield: quantitative; LCMS (RT): 2.6 min (Method D); MS (ES+) gave m/z: 233.2 (MH+).

### 31(F) (4-Fluoro-phenyl)-{(S)-3-[3-(4-methyl-1H-pyrrol-2-yl)-[1,2,4]oxadiazol-5-yl]-piperidin-1-yl}-methanone

The compound was prepared following the procedure described in the Example 1(C), starting from (S)-3-[3-(4-methyl-1H-pyrrol-2-yl)-[1,2,4]oxadiazol-5-yl]-piperidine trifluoroacetate and using 4-fluorobenzoyl chloride as the acylating agent. The final compound was purified by flash chromatography (silica gel cartridge, eluent gradient: from hexane/ethyl acetate 100:0 to hexane/ethyl acetate 60:40).

Yield: 60% (off-white solid); [α]_{D}²⁰=+114 (c=0.4, MeOH); mp=188-190° C.; LCMS (RT): 7.01 min (Method C); MS (ES+) gave m/z: 355.2 (MH+).

¹H-NMR (DMSO-d₆, 343K), δ (ppm): 11.15 (s br, 1H); 7.46 (dd, 2H); 7.23 (dd, 2H); 6.73 (m, 1H); 6.55 (m, 1H); 4.21 (m, 1H); 3.76 (m, 1H); 3.48 (dd, 1H); 3.38-3.19 (m, 2H); 2.23 (m, 1H); 2.07 (s, 3H); 2.01-1.76 (m, 2H); 1.64 (m, 1H).

### Example 32

### (3,4-Difluoro-phenyl)-{(S)-3-[3-(4-methyl-1H-pyrrol-2-yl)-[1,2,4]oxadiazol-5-yl]-piperidin-1-yl}-methanone

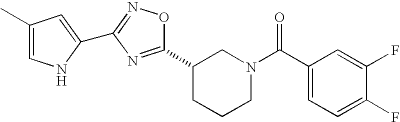

The compound was prepared following the procedure described in the Example 1 (C), starting from (S)-3-[3-(4-methyl-1H-pyrrol-2-yl)-[1,2,4]oxadiazol-5-yl]-piperidine trifluoroacetate, prepared as described in Example 31 (E), and using 3,4-difluorobenzoyl chloride as the acylating agent. The final compound was purified by flash chromatography (silica gel, eluent gradient: from hexane/ethyl acetate 100:0 to hexane/ethyl acetate 40:60).

Yield: 77% (white solid); [α]_{D}²⁰=+107 (c=0.5, MeOH); mp=166-167° C.; LCMS (RT): 3.02 min (Method N); MS (ES+) gave m/z: 373.1 (MH+).

¹H-NMR (DMSO-d₆, 353K), δ (ppm): 11.09 (s br, 1H); 7.51-7.38 (m, 2H); 7.26 (m, 1H); 6.73 (m, 1H); 6.56 (m, 1H); 4.18 (m, 1H); 3.73 (dt, 1H); 3.51 (dd, 1H); 3.40-3.24 (m, 2H); 2.23 (m, 1H); 2.08 (s, 3H); 2.02-1.75 (m, 2H); 1.65 (m, 1H).

### Example 33

### (6-Fluoro-pyridin-3-yl)-{(S)-3-[3-(4-methyl-1H-pyrrol-2-yl)-[1,2,4]oxadiazol-5-yl]-piperidin-1-yl}-methanone

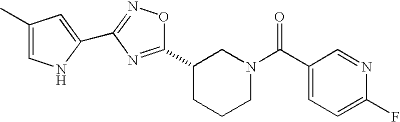

The compound was prepared following the procedure described in the Example 28 (C), starting from (S)-3-[3-(4-methyl-1H-pyrrol-2-yl)-[1,2,4]oxadiazol-5-yl]-piperidine trifluoroacetate, prepared as described in Example 31 (E), and using 6-fluoro-nicotinic acid as the acid of choice. The final compound was purified by flash chromatography (silica gel, eluent gradient: from hexane/ethyl acetate 100:0 to hexane/ethyl acetate 0:100).

Yield: 93% (white solid); [α]_{D}²⁰=+131 (c=0.5, MeOH); LCMS (RT): 2.58 min (Method N); MS (ES+) gave m/z: 356.1 (MH+).

¹H-NMR (DMSO-d₆, 353K), (ppm): 11.16 (s br, 1H); 8.31 (m, 1H); 8.02 (ddd, 1H); 7.22 (dd, 1H); 6.74 (m, 1H); 6.56 (m, 1H); 4.21 (m, 1H); 3.76 (m, 1H); 3.54 (dd, 1H); 3.43-3.27 (m, 2H); 2.22 (m, 1H); 2.08 (s, 3H); 2.03-1.75 (m, 2H); 1.66 (m, 1H).

### Example 34

### (2-Fluoro-pyridin-4-yl)-{(S)-3-[3-(4-methyl-1H-pyrrol-2-yl)-[1,2,4]oxadiazol-5-yl]-piperidin-1-yl}-methanone

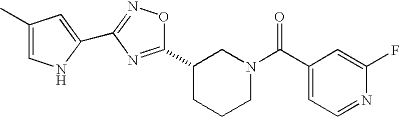

The compound was prepared following the procedure described in the Example 28 (C), starting from (S)-3-[3-(4-methyl-1H-pyrrol-2-yl)-[1,2,4]oxadiazol-5-yl]-piperidine trifluoroacetate, prepared as described in Example 31 (E), and using 2-fluoro-isonicotinic acid as the acid of choice. The final compound was purified by flash chromatography (silica gel, eluent gradient: from hexane/ethyl acetate 100:0 to hexane/ethyl acetate 1:1).

Yield: 49% (white glass); [α]_{D}²⁰1=+113 (c=0.67, MeOH); LCMS (RT): 3.68 min (Method P); MS (ES+) gave m/z: 356.4 (MH+).

¹H-NMR (DMSO-d_{6,} 353K), δ (ppm): 11.15 (s br, 1H); 8.32 (m, 1H); 7.34 (ddd, 1H); 7.16 (m, 1H); 6.74 (m, 1H); 6.56 (m, 1H); 4.18 (m br, 1H); 3.69 (m br, 1H); 3.53 (dd, 1H); 3.43-3.24 (m, 2H); 2.22 (m, 1H); 2.08 (s, 3H); 2.03-1.75 (m, 2H); 1.67 (m, 1H).

### Example 35

### (5-Methyl-isoxazol-4-yl)-{(S)-3-[3-(4-methyl-1H-pyrrol-2-yl)-[1,2,4]oxadiazol-5-yl]-piperidin-1-yl}-methanone

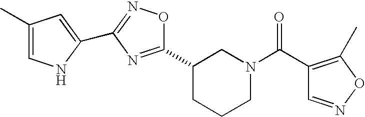

The compound was prepared following the procedure described in the Example 28 (C), starting from (S)-3-[3-(4-methyl-1H-pyrrol-2-yl)-[1,2,4]oxadiazol-5-yl]-piperidine trifluoroacetate, prepared as described in Example 31 (E), and using 5-methyl-isoxazole-4-carboxylic acid as the acid of choice. The final compound was purified by flash chromatography (silica gel, eluent gradient: from hexane/ethyl acetate 100:0 to hexane/ethyl acetate 1:1).

Yield: 68% (colourless gum); [α]_{D}²⁰=+102.5 (c=0.62, MeOH); LCMS (RT): 2.5 min (Method N); MS (ES+) gave m/z: 342.3 (MH+).

¹H-NMR (DMSO-d_{6,} 353K), δ (ppm): 11.15 (s br, 1H); 8.58 (m, 1H); 6.74 (m, 1H); 6.56 (m, 1H); 4.22 (m, 1H); 3.78 (dt, 1H); 3.54 (dd, 1H); 3.42-3.27 (m, 2H); 2.46 (d, 3H); 2.22 (m, 1H); 2.08 (m, 3H); 2.03-1.76 (m, 2H); 1.65 (m, 1H).

### Example 36

### (4-Fluoro-phenyl)-{(S)-3-[5-(4-nitro-1H-pyrrol-2-yl)-[1,2,4]oxadiazol-3-yl]-piperidin-1-yl}-methanone

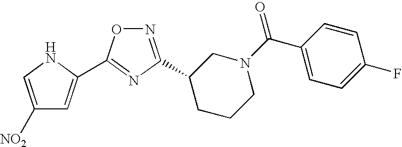

A mixture of 4-nitro-pyrrole-2-carboxylic acid (200 mg, 1.28 mmol), EDCI.HCl (370 mg, 1.92 mmol) and HOAT (175 mg, 1.28 mmol) in dioxane (70 mL) was stirred at 50° C. for 1 h, then (S)-1-(4-fluoro-benzoyl)-N-hydroxy-piperidine-3-carboxamidine (340 mg, 1.28 mmol), prepared as described in Example 27 (D), was added and the mixture was stirred at 80° C. overnight, then for a weekend at room temperature and then under reflux for 20 h. Solvent was removed. The residue was diluted with ethyl acetate and water, the phases were separated and the organic layer was washed with Na₂CO₃ (aq), dried over Na₂SO₄ and concentrated under reduced pressure. Purification of the crude by flash chromatography (silica gel, eluent gradient: from DCM/MeOH 99:1 to DCM/MeOH 97:3) gave a solid that was triturated from diisopropylether.

Yield: 34% (White powder); [α]_{D}²⁰=+92.8 (c=0.91 MeOH); mp=157-158° C.; LCMS (RT): 6.47 min (Method Q); MS (ES+) gave m/z: 386.1 (MH+).

¹H-NMR (DMSO-d_{6,} 368K), δ (ppm): 13.10 (s br, 1H); 8.02 (d, 1H); 7.45 (dd, 2H); 7.43 (m, 1H); 7.20 (dd, 2H); 4.26 (m, 1H); 3.82 (m, 1H); 3.38 (dd, 1H); 3.23 (ddd, 1H); 3.14 (m, 1H); 2.27-2.16 (m, 1H); 1.99-1.77 (m, 2H); 1.71-1.55 (m, 1H).

### Example 37

### (4-Fluoro-phenyl)-{(R)-3-[3-(1H-pyrrol-2-yl)-[1,2,4]oxadiazol-5-yl]-piperidin-1-yl}-methanone

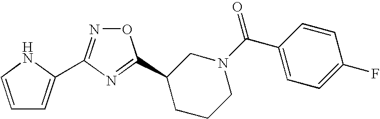

The title compound was prepared following the experimental procedure described in Example 1, starting from 1H-pyrrole-2-carbonitrile and using (R)-N-Boc-nipecotic acid. Purification of the final compound was performed by flash chromatography (silica gel, eluent gradient: from hexane/ethyl acetate 7:3 to hexane/ethyl acetate 1:1). The resulting colourless oil was triturated with diisopropylether to give (4-fluoro-phenyl)-{(R)-3-[3-(1H-pyrrol-2-yl)-[1,2,4]oxadiazol-5-yl]-piperidin-1-yl}-methanone as a white solid.

Yield: 47% (White powder); [α]_{D}²⁰=−125.7 (c=0.98, MeOH); mp=132-133° C.;

LCMS (RT): 6.71 min (Method C); MS (ES+) gave m/z: 341.1 (MH+).

¹H-NMR (DMSO-d₆), δ (ppm): 11.54 (s br, 1H); 7.46 (dd, 2H); 7.23 (dd, 2H); 6.97 (m, 1H); 6.74 (m, 1H); 6.21 (m, 1H); 4.22 (m, 1H); 3.77 (m, 1H); 3.50 (dd, 1H); 3.39-3.21 (m, 2H); 2.24 (m, 1H); 2.02-1.75 (m, 2H); 1.63 (m, 1H).

### Example 38

### (4-Fluoro-phenyl)-{(S)-3-[5-(5-methyl-1H-pyrrol-2-yl)-[1,2,4]oxadiazol-3-yl]-piperidin-1-yl}-methanone

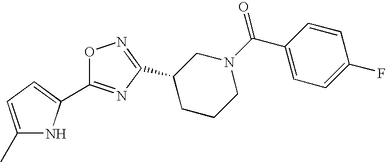

### 38(A) 5-Methyl-1H-pyrrole-2-carboxylic Acid

A solution of 5-methyl-1H-pyrrole-2-carboxylic acid ethyl ester (400 mg, 2.61 mmol), prepared as described in Curran, T.; Keaney, M.; J. Org. Chem., 61 (25), 1996, 9068-9069, and sodium hydroxide (520 mg, 13 mmol) in dioxane/water/ethanol (10 mL/1 mL/2 mL) was refluxed for 3 h. The solvent was removed and the crude was partitioned between water and DCM. 1N HCl was added to adjust the pH to 1 and the phases were separated. The organic layer was dried over sodium sulphate and evaporated under vacuum to give a solid that was used for the next step without further purification.

Yield: quantitative; LCMS (RT): 2.51 min (Method D); MS (ES+) gave m/z: 126.03 (MH+).

### 38(B) (4-Fluoro-phenyl)-{(S)-3-[5-(5-methyl-1H-pyrrol-2-yl)-[1,2,4]oxadiazol-3-yl]-piperidin-1-yl}-methanone

A mixture of 5-methyl-1H-pyrrole-2-carboxylic acid (236 mg, 1.89 mmol), (S)-1-(4-fluoro-benzoyl)-N-hydroxy-piperidine-3-carboxamidine (500 mg, 1.89 mmol), prepared as described in Example 27 (D), EDCI.HCl (543 mg, 2.84 mmol) and HOAT (257 mg, 1.89 mmol) in DCM (15 mL) was stirred at room temperature overnight, then the solvent was removed and the residue was dissolved in dioxane and refluxed for 24 h. Solvent was removed and the residue was diluted with ethyl acetate and water, the phases were separated and the organic layer was washed with Na₂CO₃ (aq), then with 1N HCl, dried over Na₂SO₄ and concentrated under reduced pressure. Purification of the crude was performed by preparative HPLC.

Yield: 1% (black oil); LCMS (RT): 7.41 min (Method C); MS (ES+) gave m/z: 355.2° (MH+).

¹H-NMR (DMSO-d₆ 343K), δ (ppm): 10.86 (s br, 1H); 8.15 (dd, 2H); 7.44 (dd, 2H); 6.39 (m, 1H); 5.82 (m, 1H); 4.56 (m, 1H); 4.23 (m, 1H); 3.44-3.18 (m, 2H); 3.09 (m, 1H); 2.24 (m, 1H); 2.20 (s, 3H); 1.99-1.80 (m, 2H); 1.61 (m, 1H).

### Example 39

### {(S)-3-[5-(4-Chloro-1H-pyrrol-2-yl)-[1,2,4]oxadiazol-3-yl]-piperidin-1-yl}-(4-fluoro-phenyl)-methanone

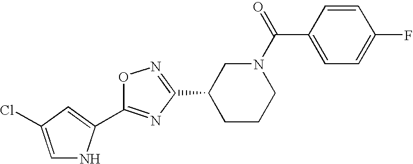

### 39(A) 4-Chloro-1H-pyrrole-2-carboxylic Acid

A mixture of 2,2,2-trichloro-1-(4-chloro-1H-pyrrol-2-yl)-ethanone (14.12 mmol), prepared as described in Belanger; Tetrahedron Lett.; 1979; 2505-2508, and 5 mL of 10% NaOH (aq) in THF (10 mL) was stirred at room temperature for 1 h. The solvent was removed and the crude was partitioned between water and ethyl acetate, then 10% HCl was added to adjust the pH to 5. The phases were separated, the aqueous layer was re-extracted with ethyl acetate, the combined organics were dried over magnesium sulphate. After evaporation, 4-Chloro-1H-pyrrole-2-carboxylic acid was obtained as a solid, which was used for the next step without further purification.

Yield: quantitative; LCMS (RT): 3.3 min (Method D); MS (ES+) gave m/z: 145.9 and 147.9 (MH+).

### 39(B) (S)-3-[5-(4-Chloro-1H-pyrrol-2-yl)-[1,2,4]oxadiazol-3-yl]-piperidine-1-carboxylic Acid tert-butyl Ester

A mixture of 4-chloro-1H-pyrrole-2-carboxylic acid (769 mg, 5.28 mmol), (S)-3-(N-hydroxycarbamimidoyl)-piperidine-1-carboxylic acid tert-butyl ester (4.8 mmol), prepared as described in Example 10 (C), EDCI.HCl (1.38 g, 7.2 mmol) and HOAT (653 mg, 4.8 mmol) in dioxane (15 mL) was stirred at room temperature overnight. Solvent was removed and the residue was diluted with ethyl acetate and water, the phases were separated and the organic layer was washed with 1M NaOH (aq), then dried over Na₂SO₄ and concentrated under reduced pressure.

The residue was dissolved in acetonitrile (2 mL), in the presence of few 4 A molecular sieves, and heated at 100° C. for 50 min, in a sealed tube, in a microwaves oven. The solvent was removed and the crude was passed through a silica gel short pad (eluent: petroleum ether/ethyl acetate 2:1) to afford (S)-3-[5-(4-chloro-1H-pyrrol-2-yl)-[1,2,4]oxadiazol-3-yl]-piperidine-1-carboxylic acid tert-butyl ester (250 mg).

Yield: 73% (yellow oil); LCMS (RT): 5.42 min (Method E); MS (ES+) gave m/z: 353.08 (MH+).

### 39(C) (S)-3-[5-(4-Chloro-1H-pyrrol-2-yl)-[1,2,4]oxadiazol-3-yl]-piperidine Hydrochloride

To a solution of (S)-3-[5-(4-methyl-1H-pyrrol-2-yl)-[1,2,4]oxadiazol-3-yl]-piperidine-1-carboxylic acid tert-butyl ester (0.25 g, 0.71 mmol) in dichloromethane (10 mL), 1.7 mL of 4N HCl (dioxane solution) were added at 0° C. and the reaction mixture was allowed to warm at room temperature and stirred for 3 h. The solvent was evaporated under reduced pressure to give the title compound, which was used for the next step without further purification.

Yield: 92%; LCMS (RT): 3.0 min (Method D); MS (ES+) gave m/z: 253.1 (MH+).

### 39(D) {(S)-3-[5-(4-Chloro-1H-pyrrol-2-yl)-[1,2,4]oxadiazol-3-yl]-piperidin-1-yl}-(4-fluoro-phenyl)-methanone

The compound was prepared following the procedure described in the Example 1(C), starting from (S)-3-[5-(4-chloro-1H-pyrrol-2-yl)-[1,2,4]oxadiazol-3-yl]-piperidine hydrochloride, prepared as described in Example 39 (C), and using 4-fluorobenzoyl chloride as the acylating agent. The final compound was purified by flash chromatography (silica gel, eluent: petroleum ether/ethyl acetate 1:2).

Yield: 79% (white solid); LCMS (RT): 3.00 min (Method N); MS (ES+) gave m/z: 375.2 (MH+).

¹H-NMR (DMSO-d₆ 353K), δ (ppm): 7.46 (dd, 2H); 7.22 (dd, 2H); 7.20 (m, 1H); 6.94 (d, 1H); 4.25 (m, 1H); 3.83 (m, 1H); 3.33 (dd, 1H); 3.20 (ddd, 1H); 3.09 (m, 1H); 2.19 (m, 1H); 1.96-1.76 (m, 2H); 1.62 (m, 1H).

### Example 40

{(S)-3-[5-(4-Chloro-1H-pyrrol-2-yl)-[1,2,4]oxadiazol-3-yl]-piperidin-1-yl}-(6-fluoro-pyridin-3-yl)-methanone

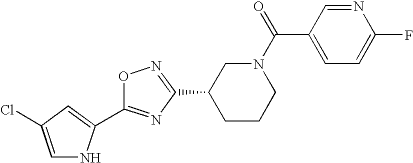

The title compound was prepared following the experimental procedure described in Example 28(C), starting from (S)-3-[5-(4-chloro-1H-pyrrol-2-yl)-[1,2,4]oxadiazol-3-yl]-piperidine hydrochloride, prepared as described in Example 39 (C), and using 6-fluoro-nicotinic acid as the acid of choice.

Purification was performed by flash chromatography (silica gel, eluent: petroleum ether/ethyl acetate 1:2).

Yield: 82% (White solid); [α]_{D}²⁰=+109.8 (c=1.08, MeOH); LCMS (RT): 2.69 min (Method N); MS (ES+) gave m/z: 376.3 (MH+).

¹H-NMR (DMSO-d₆ 353K), δ (ppm): 12.37 (s br, 1H); 8.31 (m, 1H); 8.02 (ddd, 1H); 7.23-7.18 (m, 2H); 6.94 (d, 1H); 4.24 (m, 1H); 3.81 (m, 1H); 3.38 (dd, 1H); 3.27 (ddd, 1H); 3.14 (m, 1H); 2.20 (m, 1H); 1.98-1.76 (m, 2H); 1.66 (m, 1H).

### Example 41

### {(S)-3-[5-(4-Chloro-1H-pyrrol-2-yl)-[1,2,4]oxadiazol-3-yl]-piperidin-1-yl}-(2-fluoro-pyridin-4-yl)-methanone

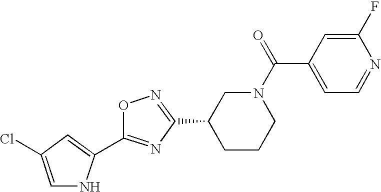

The title compound was prepared following the experimental procedure described in Example 28(C), starting from (S)-3-[5-(4-chloro-1H-pyrrol-2-yl)-[1,2,4]oxadiazol-3-yl]-piperidine hydrochloride, prepared as described in Example 39 (C), and using 2-fluoro-pyridine-4-carboxylic acid as the acid of choice.

Purification was performed by flash chromatography (silica gel, eluent: petroleum ether/ethyl acetate 1:2).

Yield: 86% (White solid); [α]_{D}²⁰=+94.5 (c=0.92, MeOH); LCMS (RT): 2.69 min (Method N); MS (ES+) gave m/z: 376.2 (MH+).

¹H-NMR (DMSO-d₆ 373K), δ (ppm): 12.24 (s br, 1H); 8.31 (m, 1H); 7.32 (ddd, 1H); 7.18 (d, 1H); 7.13 (m, 1H); 6.93 (d, 1H); 4.19 (m, 1H); 3.74 (m, 1H); 3.39 (dd, 1H); 3.26 (ddd, 1H); 3.15 (m, 1H); 2.20 (m, 1H); 1.98-1.76 (m, 2H); 1.67 (m, 1H).

### Example 42

### {(S)-3-[5-(4-Chloro-1H-pyrrol-2-yl)-[1,2,4]oxadiazol-3-yl]-piperidin-1-yl}-(5-methyl-isoxazol-4-yl)-methanone

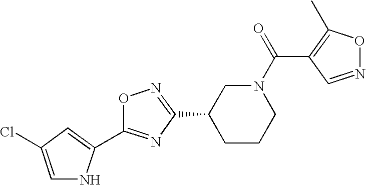

The title compound was prepared following the experimental procedure described in Example 28(C), starting from (S)-3-[5-(4-chloro-1H-pyrrol-2-yl)-[1,2,4]oxadiazol-3-yl]-piperidine hydrochloride, prepared as described in Example 39 (C), and using 5-methyl-isoxazole-4-carboxylic acid as the acid of choice.

Purification was performed by flash chromatography (silica gel, eluent: petroleum ether/ethyl acetate 1:2).

Yield: 91% (White solid); [α]_{D}²⁰=+90.2 (c=1.05, MeOH); LCMS (RT): 2.63 min (Method N); MS (ES+) gave m/z: 362.2 (MH+).

¹H-NMR (DMSO-d₆ 373K), δ (ppm): 12.27 (s br, 1H); 8.53 (m, 1H); 7.18 (d, 1H); 6.94 (d, 1H); 4.25 (m, 1H); 3.84 (m, 1H); 3.39 (dd, 1H); 3.28 (ddd, 1H); 3.10 (m, 1H); 2.47 (d, 3H); 2.20 (m, 1H); 1.98-1.79 (m, 2H); 1.64 (m, 1H).

### Example 43

### {(S)-3-[3-(4-Chloro-1H-pyrrol-2-yl)-[1,2,4]oxadiazol-5-yl]-piperidin-1-yl}-(4-fluoro-phenyl)-methanone

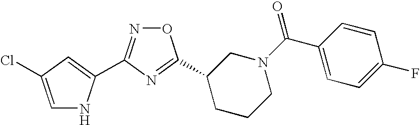

### 43(A) 4-Chloro-1H-pyrrole-2-carboxylic Acid Amide

A solution of 2,2,2-trichloro-1-(4-chloro-1H-pyrrol-2-yl)-ethanone (7.6 mmol), prepared as described in Belanger; Tetrahedron Lett.; 1979; 2505-2508, and conc. NH₄OH (15 mL) in acetonitrile (15 mL) was refluxed for 10 min. The solvent was removed and the crude was partitioned between water and ethyl acetate, the organic layer was then dried over sodium sulphate and evaporated under reduced pressure. The crude was purified by flash chromatography (silica gel, eluent: petroleum ether/ethyl acetate 4:6).

Yield: 100%; LCMS (RT): 2.37 min (Method D); MS (ES+) gave m/z: 145.17 (MH+).

### 43(B) 4-Chloro-1H-pyrrole-2-carbonitrile

A solution of 4-chloro-1H-pyrrole-2-carboxylic acid amide (570 mg, 3.94 mmol) and phosphorus oxychloride (370 μL, 3.94 mmol) in pyridine (10 mL) was stirred at room temperature overnight, then the mixture was diluted with ethyl acetate and washed with 10% HCl (twice). The organic layer was dried over sodium sulphate and evaporated under reduced pressure to give a crude that was purified by flash chromatography (silica gel, eluent: petroleum ether/ethyl acetate 9:1).

Yield: 22%; LCMS (RT): 3.97 min (Method D); MS (ES+) gave m/z: 127.13 (MH+).

### 43(C) 4-Chloro-N-hydroxy-1H-pyrrole-2-carboxamidine

The compound was prepared following the same experimental procedure described in Example 31 (C), starting from 4-chloro-1H-pyrrole-2-carbonitrile.

Yield: 100%; LCMS (RT): 0.71 min (Method D); MS (ES+) gave m/z: 160.21 (MH+).

### 43(D) (S)-3-[3-(4-Chloro-1H-pyrrol-2-yl)-[1,2,4]oxadiazol-5-yl]-piperidine-1-carboxylic Acid tert-butyl Ester

A mixture of (S)-N-Boc-nipecotic acid (199 mg, 0.87 mmol), 4-chloro-N-hydroxy-1H-pyrrole-2-carboxamidine (0.87 mmol), HOAT (119 mg, 0.87 mmol), EDCI.HCl (250 mg, 1.305 mmol) in dry dioxane (10 mL) was heated at 80° C. for 16 h, under nitrogen atmosphere. The solvent was removed under vacuum, the residue was partitioned between water and ethyl acetate, the phases were separated. The organic layer was dried over sodium sulphate to give a residue that was purified by flash chromatography (silica gel, eluent: petroleum ether/ethyl acetate 8:2).

Yield: 20%; LCMS (RT): 6.03 min (Method D); MS (ES+) gave m/z: 353.0 (MH+).

### 43(E) (S)-3-[3-(4-Chloro-1H-pyrrol-2-yl)-[1,2,4]oxadiazol-5-yl]-piperidine Hydrochloride

To a solution of (S)-3-[3-(4-chloro-1H-pyrrol-2-yl)-[1,2,4]oxadiazol-5-yl]-piperidine-1-carboxylic acid tert-butyl ester (60 mg, 0.17 mmol) in dichloromethane (2 mL), 1.0 mL of 4N HCl (dioxane solution) was added at 0° C. and the reaction mixture was allowed to warm at room temperature and stirred for 1 h. The solvent was evaporated under reduced pressure to give the title compound, which was used for the next step without further purification.

Yield: quantitative; LCMS (RT): 2.68 min (Method D); MS (ES+) gave m/z: 253.28 (MH+).

### 43(F) {(S)-3-[3-(4-Chloro-1H-pyrrol-2-yl)-[1,2,4]oxadiazol-5-yl]-piperidin-1-yl}-(4-fluoro-phenyl)-methanone

The compound was prepared following the procedure described in the Example 1(C), starting from (S)-3-[3-(4-chloro-1H-pyrrol-2-yl)-[1,2,4]oxadiazol-5-yl]-piperidine hydrochloride, prepared as described in Example 43 (E), and using 4-fluorobenzoyl chloride as the acylating agent. The final compound was purified by preparative HPLC.

Yield: 31% (pink solid); [α]_{D}²⁰=+114.1 (c=0.80, CH₃OH); LCMS (RT): 6.01 min (Method R); MS (ES+) gave m/z: 375.1 (MH+).

¹H-NMR (DMSO-d₆ 353K), δ (ppm): 11.83 (s br, 1H); 7.45 (dd, 2H); 7.22 (dd, 2H); 7.03 (dd, 1H); 6.69 (dd, 1H); 4.22 (m, 1H); 3.75 (m, 1H); 3.51 (dd, 1H); 3.41-3.19 (m, 2H); 2.24 (m, 1H); 2.04-1.75 (m, 2H); 1.64 (m, 1H).

### Example 44

### {(S)-3-[5-(4-Bromo-1H-pyrrol-2-yl)-[1,2,4]oxadiazol-3-yl]-piperidin-1-yl}-(6-fluoro-pyridin-3-yl)-methanone

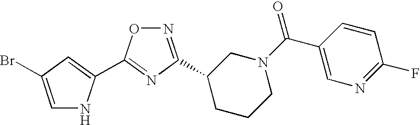

### 44 (A) (S)-1-(6-Fluoro-pyridine-3-carbonyl)-N-hydroxy-piperidine-3-carboxamidine

(S)-3-Cyano-piperidine-1-carboxylic acid tert-butyl ester (2.33 g, 11.1 mmol), prepared as described in Example 27 (B), was dissolved in DCM (15 mL) and 9 mL of HCl 4N (dioxane solution) were added dropwise at 0° C. The resulting mixture was stirred at room temperature for 1.5 h. The solvent was evaporated under reduced pressure to afford (S)-piperidine-3-carbonitrile hydrochloride as a white solid, that was used for the next step without further purification.

A mixture of (S)-piperidine-3-carbonitrile hydrochloride (11.1 mmol) 6-fluoro-nicotinic acid (1.6 g, 11.1 mmol), HOBT (2.24 g, 16.6 mmol), EDCI.HCl (2.13 g, 11.1 mmol) and triethylamine (3.1 mL, 22.2 mmol) in dry DCM (20 mL) was kept under stirring at RT overnight, under nitrogen atmosphere. The mixture was diluted with DCM and was washed sequentially with 5% Na₂CO₃ (aq) (10 mL, twice) and with brine. The organic layer was dried over sodium sulphate and the solvent was removed under vacuum to give a residue that was purified by flash chromatography (silica gel, eluent: DCM/MeOH 98:2) to give 1.36 g of (S)-1-(6-fluoro-pyridine-3-carbonyl)-piperidine-3-carbonitrile.

A solution of (S)-1-(6-fluoro-pyridine-3-carbonyl)-piperidine-3-carbonitrile (150 mg, 0.64 mmol) and aqueous hydroxylamine (50% in water, 160 uL, 2.6 mmol) in ethanol (5 mL) was refluxed for 4 h. The solvent was evaporated under reduced pressure to afford the title compound that was used for the next step without further purification.

Yield: quantitative; HPLC (RT): 1.48 min (Method F).

### 44(B) 4-Bromo-1H-pyrrole-2-carboxylic Acid

A solution of 1-(4-bromo-1H-pyrrol-2-yl)-2,2,2-trichloro-ethanone (4.7 mmol), prepared as described in Belanger; Tetrahedron Lett.; 1979; 2505-2508, and 1 mL of 10% NaOH (aq) in THF (5 mL) was stirred at room temperature for 1 h. The solvent was removed and the crude was partitioned between water and ethyl acetate, then 10% HCl was added to adjust the pH to 5. The phases were separated, the aqueous layer was re-extracted with ethyl acetate, the combined organics were dried over magnesium sulphate. After evaporation, 4-bromo-1H-pyrrole-2-carboxylic acid was obtained as a solid, which was used for the next step without further purification.

Yield: 64%; LCMS (RT): 2.74 min (Method B); MS (ES+) gave m/z: 191 and 193.

### 44 (C) {(S)-3-[5-(4-Bromo-1H-pyrrol-2-yl)-[1,2,4]oxadiazol-3-yl]-piperidin-1-yl}-(6-fluoro-pyridin-3-yl)-methanone

A solution of 4-bromo-1H-pyrrole-2-carboxylic acid (134 mg, 0.704 mmol), (S)-1-(6-fluoro-pyridine-3-carbonyl)-N-hydroxy-piperidine-3-carboxamidine (0.64 mmol), EDC (184 mg, 0.96 mmol), HOAT (87 mg, 0.64 mmol) in dioxane (5 mL) was stirred at room temperature overnight. The solvent was removed, the crude was diluted with DCM and washed with 1N NaOH, the organic layer was dried over sodium sulphate and evaporated under reduced pressure to give a solid that was purified by flash chromatography (silica gel, eluent: DCM/MeOH 9:1). The solid obtained after this purification was dissolved in acetonitrile and heated at 110° C. for 6 h, in a sealed tube, in a microwaves oven, then another heating cycle was performed (6 h, 130° C., microwaves). The solvent was evaporated under reduced pressure and the crude was purified by preparative HPLC.

Yield: 11% (yellow oil); [α]_{D}²⁰=+95.19 (c=1.2, CH₃OH); LCMS (RT): 2.80 min (Method N); MS (ES+) gave m/z: 420.0 (MH+).

¹H-NMR (DMSO-d₆ 353K), δ (ppm): 12.36 (s br, 1H); 8.30 (m, 1H); 8.01 (ddd, 1H); 7.22 (d, 1H); 7.19 (dd, 1H); 6.99 (d, 1H); 4.23 (m, 1H); 3.80 (m, 1H); 3.39 (dd, 1H); 3.27 (ddd, 1H); 3.14 (m, 1H); 2.20 (m, 1H); 1.98-1.76 (m, 2H); 1.66 (m, 1H).

### Example 45

### {(S)-3-[3-(4-Bromo-1H-pyrrol-2-yl)-[1,2,4]oxadiazol-5-yl]-piperidin-1-yl}-(4-fluoro-phenyl)-methanone

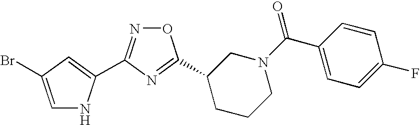

### 45 (A) (S)-3-[3-(4-Bromo-1H-pyrrol-2-yl)-[1,2,4]oxadiazol-5-yl]-piperidine Hydrochloride

The compound was prepared starting from 1-(4-bromo-1H-pyrrol-2-yl)-2,2,2-trichloro-ethanone (prepared as described in Belanger; Tetrahedron Lett.; 1979; 2505-2508) according to the experimental procedures described in Examples 43 (A), 43 (B). 43 (C). 43 (D) and 43 (E).

LCMS (RT): 2.93 min (Method D); MS (ES+) gave m/z: 297.17 (MH+).

### 45 (B) {(S)-3-[3-(4-Bromo-1H-pyrrol-2-yl)-[1,2,4]oxadiazol-5-yl]-piperidin-1-yl}-(4-fluoro-phenyl)-methanone

The compound was prepared following the procedure described in the Example 1(C), starting from (S)-3-[3-(4-bromo-1H-pyrrol-2-yl)-[1,2,4]oxadiazol-5-yl]-piperidine hydrochloride, prepared as described in Example 45 (A), and using 4-fluorobenzoyl chloride as the acylating agent. The final compound was purified by flash chromatography (silica gel, eluent: petroleum ether/ethyl acetate 7:3) and then by preparative HPLC.

Yield: 26% (white solid); [α]_{D}²⁰=+123.3 (c—0.73, CH₃OH); LCMS (RT): 6.08 min (Method R); MS (ES+) gave m/z: 419.1 (MH+).

¹H-NMR (DMSO-d₆ 353K), δ (ppm): 11.89 (s br, 1H); 7.45 (dd, 2H); 7.22 (dd, 2H); 7.06 (d, 1H); 6.75 (d, 1H); 4.22 (m, 1H); 3.75 (m, 1H); 3.51 (dd, 1H); 3.41-3.21 (m, 2H); 2.24 (m, 1H); 2.04-1.76 (m, 2H); 1.63 (m, 1H).

### Example 46

### {(S)-3-[3-(4-Bromo-1H-pyrrol-2-yl)-[1,2,4]oxadiazol-5-yl]-piperidin-1-yl}-(6-fluoro-pyridin-3-yl)-methanone

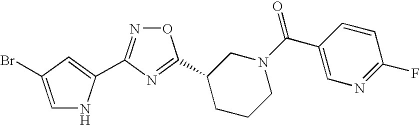

The title compound was prepared following the experimental procedure described in Example 28(C), starting from (S)-3-[3-(4-bromo-1H-pyrrol-2-yl)-[1,2,4]oxadiazol-5-yl]-piperidine hydrochloride, prepared as described in Example 45 (A), and using 6-fluoro-nicotinic acid as the acid of choice.

The final compound was purified by flash chromatography (silica gel, eluent: DCM/MeOH 99:1) and then by preparative HPLC.

Yield: 30% (white gummy solid); LCMS (RT): 2.72 min (Method N); MS (ES+) gave m/z: 419.9 (MH+).

¹H-NMR (DMSO-d₆ 353K), δ (ppm): 11.91 (s br, 1H); 8.30 (m, 1H); 8.01 (dd, 1H); 7.21 (dd, 1H); 7.06 (dd, 1H); 6.75 (dd, 1H); 4.23 (m, 1H); 3.76 (m, 1H); 3.55 (dd, 1H); 3.45-3.27 (m, 2H); 2.25 (m, 1H); 2.05-1.76 (m, 2H); 1.67 (m, 1H).

### Example 47

### (4-Fluoro-phenyl)-{3-fluoro-3-[3-(1H-pyrrol-2-yl)-[1,2,4]oxadiazol-5-yl]-piperidin-1-yl}-methanone

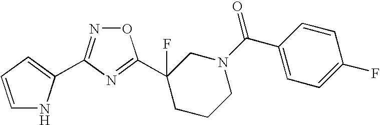

### 47(A) 1-(4-Fluoro-benzoyl)-piperidine-3-carboxylic Acid Ethyl Ester

To a cooled solution of ethyl nipecotate (0.5 mL, 3.21 mmol) in dry DCM (10 mL), 4-fluorobenzoyl chloride (380 μL, 3.21 mmol) and then triethylamine (496 μL, 3.54 mmol) were slowly added. After stirring 2 h at room temperature, solvent was removed and the residue was treated with water and ethyl acetate. The phases were separated, the organic layer was washed with 1N NaOH (twice), with 1N HCl (twice), and then with brine. The organic layer was dried over sodium sulphate and evaporated under reduced pressure to give 881 mg of an oil which was used for the next step without further purification.

Yield: 98% (oil); LCMS (RT): 4.57 min (Method D); MS (ES+) gave m/z: 280.3 (MH+).

### 47(B) 3-Fluoro-1-(4-fluoro-benzoyl)-piperidine-3-carboxylic Acid Ethyl Ester

LHMDS (1N solution in THF, 3.5 mL, 3.48 mmol) was slowly added into a solution of 1-(4-fluoro-benzoyl)-piperidine-3-carboxylic acid ethyl ester (881 mg, 3.16 mmol) in dry THF (20 mL), cooled at −78° C., under nitrogen atmosphere. The solution was stirred at −78° C. for 1 h, then a solution of N-fluoro-dibenzenesulphonimide (997 mg, 3.16 mmol) in dry THF (10 mL) was slowly added. After stirring 3 h at −78° C., the mixture was allowed to warm to room temperature and stirred at room temperature overnight. 1N HCl was slowly dropped at 0° C. Solvent was removed and the residue was treated with 1N HCl and ethyl acetate. The phases were separated and the organics were washed with 1N HCl (3 times) and with brine, then the organic layer was dried over sodium sulphate and evaporated under vacuum to give a crude oil. The oil was used for the next step without further purification.

Yield: quantitative (oil); LCMS (RT): 4.59 min (Method D); MS (ES+) gave m/z: 298.2 (MH+).

### 47(C) 3-Fluoro-1-(4-fluoro-benzoyl)-piperidine-3-carboxylic Acid

A solution of 3-fluoro-1-(4-fluoro-benzoyl)-piperidine-3-carboxylic acid ethyl ester (3.16 mmol) and NaOH (126 mg, 3.16 mmol) in water (10 mL) and ethanol (10 mL) was refluxed for 3 h. Solvent was removed. The residue aqueous layer was diluted with water, washed twice with DCM and then acidified with 6N HCl to adjust the pH to 1. The aqueous layer was extracted with DCM. The organics were washed with water, dried over sodium sulphate and evaporated under reduced pressure to give 0.3 g of yellow solid.

Yield: quantitative; LCMS (RT): 3.34 min (Method D); MS (ES+) gave m/z: 270.26 (MH+).

### 47 (D) (4-Fluoro-phenyl)-{3-fluoro-3-[3-(1H-pyrrol-2-yl)-[1,2,4]oxadiazol-5-yl]-piperidin-1-yl}-methanone

A solution of 3-fluoro-1-(4-fluoro-benzoyl)-piperidine-3-carboxylic acid (450 mg, 1.67 mmol), N-hydroxy-1H-pyrrole-2-carboxamidine (209 mg, 1.67 mmol), prepared as described in Example 1(A), HOBT (225 mg, 1.67 mmol), EDCI.HCl (480 mg, 2.5 mmol) and triethylamine (470 μL, 3.34 mmol) in dioxane (25 mL) was stirred at RT for 2 h, then was refluxed for 3 h. Solvent was removed, the crude residue was purified by flash chromatography (silica gel, eluent: DCM/ethyl acetate 20:1) to afford 135 mg of (4-fluoro-phenyl)-{3-fluoro-3-[3-(1H-pyrrol-2-yl)-[1,2,4]oxadiazol-5-yl]-piperidin-1-yl}-methanone.

Yield: 23% (white solid); mp=114.8-118° C.; LCMS (RT): 2.82 min (Method N); MS (ES+) gave m/z: 359.1 (MH+).

¹H-NMR (DMSO-d₆ 353K), δ (ppm): 11.60 (s br, 1H); 7.46 (dd, 2H); 7.25 (dd, 2H); 7.01 (ddd, 1H); 6.79 (ddd, 1H); 6.24 (ddd, 1H); 4.42 (m, 1H); 4.02-3.78 (m, 2H); 3.27 (m, 1H); 2.47-2.24 (m, 2H); 1.96-1.74 (m, 2H).

### Example 48

### {3,3-Difluoro-5-[3-(1H-pyrrol-2-yl)-[1,2,4]oxadiazol-5-yl]-piperidin-1-yl}-(4-fluoro-phenyl)-methanone

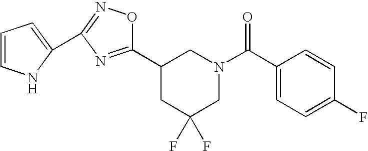

### 48(A) 5-Hydroxy-piperidine-3-carboxylic Acid Ethyl Ester

A solution of 5-hydroxy-piperidine-3-carboxylic acid (900 mg, 6.2 mmol) and

H₂SO₄ (1.5 mL) in absolute ethanol (80 mL) was stirred at room temperature overnight. The solvent was removed under reduced pressure and the crude 5-hydroxy-piperidine-3-carboxylic acid ethyl ester was used in the next step without farther purification.

Yield: 100%; LCMS (RT): 0.63 min (Method D); MS (ES+) gave m/z: 174.32 (MH+).

### 48(B) 1-(4-Fluoro-benzoyl)-5-hydroxy-piperidine-3-carboxylic Acid Ethyl Ester

A mixture of 5-hydroxy-piperidine-3-carboxylic acid ethyl ester (1.08 g, 6.2 mmol), 4-fluorobenzoic acid (870 mg, 6.2 mmol), HOAt (850 mg, 6.2 mmol), EDCI.HCl (1.78 g, 9.3 mmol) and triethylamine (8.7 mL, 62 mmol) in dry DCM (70 mL) was kept under stirring at room temperature for 3 days, under nitrogen atmosphere. The organic layer was washed with 2N HCl (1×40 mL), 5% Na₂CO₃ (aq) (1×40 mL), brine (1×40 mL) and then was dried over Na₂SO₄. The solvent was removed under vacuum to give 1-(4-fluoro-benzoyl)-5-hydroxy-piperidine-3-carboxylic acid ethyl ester that was used in the next step without further purification

Yield: 100%; LCMS (RT): 2.69 min (Method B); MS (ES+) gave m/z: 296.24 (MH+).

### 48(C) 1-(4-Fluoro-benzoyl)-5-oxo-piperidine-3-carboxylic Acid Ethyl Ester

A solution of DMSO (120 μL, 1.65 mmol) in dry DCM (15 mL) was cooled at −78° C. under nitrogen atmosphere. Oxalyl chloride (140 μL, 1.5 mmol) was added and the mixture was stirred at −78° C. for 15 min, then 1-(4-fluoro-benzoyl)-5-hydroxy-piperidine-3-carboxylic acid ethyl ester (300 mg, 1.02 mmol) was added. The mixture was stirred at −78° C. for 3 h then triethylamine (425 μL, 3.05 mmol) was added. Stirring at −78° C. was maintained for 30 min then the reaction was allowed to warm to room temperature. DCM (30 mL) was added and the solution was washed with 5% citric acid solution (2×40 mL), then solvent was removed under reduced pressure and the crude 1-(4-fluoro-benzoyl)-5-oxo-piperidine-3-carboxylic acid ethyl ester was used in the next step without further purification.

Yield: 63%; LCMS (RT): 2.72 min (Method B); MS (ES+) gave m/z: 294.24 (MH+).

### 48(D) 5,5-Difluoro-1-(4-fluoro-benzoyl)-piperidine-3-carboxylic Acid Ethyl Ester

A solution of 1-(4-fluoro-benzoyl)-5-oxo-piperidine-3-carboxylic acid ethyl ester (189 mg, 0.64 mmol) in dry DCM (15 mL) was cooled at −78° C. under nitrogen atmosphere. DAST (700 μL, 5.2 mmol) was added, the reaction was allowed to warm to room temperature, then stirring was maintained overnight. DCM (30 mL) was added and the solution was washed with 5% NaHCO₃ (aq) (2×40 mL). The organic layer was dried over Na₂SO₄, then solvent was removed under reduced pressure and the crude 5,5-difluoro-1-(4-fluoro-benzoyl)-piperidine-3-carboxylic acid ethyl ester was used in the next step without further purification.

Yield: 96%; LCMS (RT): 3.29 min (Method B); MS (ES+) gave m/z: 316.22 (MH+).

### 48(E) 5,5-Difluoro-1-(4-fluoro-benzoyl)-piperidine-3-carboxylic Acid

A solution of 5,5-difluoro-1-(4-fluoro-benzoyl)-piperidine-3-carboxylic acid ethyl ester (194 mg, 0.61 mmol) and NaOH (50 mg, 1.22 mmol) in dioxane/H₂O 10/1 (33 mL) was stirred at room temperature for 3 h, then the solvent was removed under reduced pressure. The crude residue was dissolved in H₂O then 5% HCl was added to adjust the pH to 2. The aqueous phase was extracted with AcOEt (3×10 mL), then the combined organic layers were dried over Na₂SO₄ and the solvent was removed under reduced pressure. The crude 5,5-difluoro-1-(4-fluoro-benzoyl)-piperidine-3-carboxylic acid was used in the next step without further purification.

Yield: 95%; LCMS (RT): 2.81 min (Method B); MS (ES+) gave m/z: 288.18 (MH+).

### 48 (F) {3,3′-Difluoro-5-[3-(1H-pyrrol-2-yl)-[1,2,4]oxadiazol-5-yl]-piperidin-1-yl}-(4-fluoro-phenyl)-methanone

A solution of 1H-pyrrole-2-carbonitrile (4.6 mL, 54.3 mmol) and hydroxylamine (50% aq. sol., 13.3 mL, 217.2 mmol) in ethanol (150 mL) was refluxed for 4 h, then the solvent was removed under reduced pressure to give N-Hydroxy-1H-pyrrole-2-carboxamidine. A mixture of 5,5-difluoro-1-(4-fluoro-benzoyl)-piperidine-3-carboxylic acid (167 mg, 0.58 mmol), HOAT (80 mg, 0.58 mmol) and EDCI.HCl (165 mg, 0.87 mmol) in dioxane (60 mL) was stirred at 50° C. for 2 h, then N-hydroxy-1H-pyrrole-2-carboxamidine (80 mg, 0.58 mmol) was added and the mixture was stirred at room temperature for 3 days, then at 80° C. overnight.

The solvent was removed under reduced pressure then the crude was partitioned between AcOEt and H₂O. The two layers were separated and the organic layer was washed with 5% Na₂CO₃ (aq) (2×10 mL), with brine (1×10 mL) and then was dried over Na₂SO₄. The solvent was removed under reduced pressure, then the crude was purified by flash chromatography (silica gel, eluent: hexane/ethyl acetate 70:30) and by preparative HPLC.

Yield: 14% (White powder); LCMS (RT): 2.9 min (Method N); MS (ES+) gave m/z: 377.0 (MH+).

¹H-NMR (DMSO-d₆ 353K), δ (ppm): 11.48 (s br, 1H); 7.54 (dd, 2H); 7.28 (dd, 2H); 6.96 (ddd, 1H); 6.75 (ddd, 1H); 6.22 (ddd, 1H); 4.40 (m, 1H); 4.15 (m, 1H); 3.77-3.50 (m, 3H); 2.80-2.56 (m, 2H).

### Example 49

### {3,3-Dimethyl-5-[3-(1H-pyrrol-2-yl)-[1,2,4]oxadiazol-5-yl]-piperidin-1-yl}-(4-fluoro-phenyl)-methanone

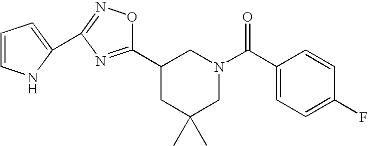

### 49(A) 3,3-Dimethyl-4-oxo-piperidine-1-carboxylic Acid tert-butyl Ester

A solution of 4-oxo-piperidine-1-carboxylic acid tert-butyl ester (500 mg, 4.2 mmol) in dry THF (10 mL) was cooled to 10° C. under nitrogen atmosphere. NaH (403 mg, 9.2 mmol) and CH₃I (664 μL, 10.5 mmol) were added and the mixture was stirred at 10° C. for 30 min. The solvent was removed under reduced pressure and the crude was partitioned between diethyl ether and brine. The two layers were separated and the organic layer was dried over Na₂SO₄. The solvent was removed under reduced pressure and the crude 3,3-dimethyl-4-oxo-piperidine-1-carboxylic acid tert-butyl ester was used in the next step without further purification

Yield: 73%; ¹H-NMR (CDCl₃, 300 MHz): 1.05 (s, 6H), 1.45 (s, 9H), 2.50 (t, 2H), 3.40 (s, 2H), 3.75 (t, 2H).

### 49(B) 5,5-Dimethyl-4-oxo-piperidine-1,3-dicarboxylic Acid 1-tert-butyl Ester 3-methyl Ester

A solution of 3,3-dimethyl-4-oxo-piperidine-1-carboxylic acid tert-butyl ester (1.8 g, 7.9 mmol) in dry THF (30 mL) was cooled to −78° C. under nitrogen atmosphere. LHMDS (1M in THF, 9.5 mL, 9.5 mmol) was added, stirring was maintained at −78° C. for 1 h, then CNCO₂Me (752 μL, 9.5 mmol) was slowly added. The mixture was stirred at −78° C. for 10 min, then H₂O (30 mL) was added. The reaction was allowed to warm to room temperature. THF was removed under reduced pressure, then the aqueous phase was extracted with ethyl acetate (3×30 mL). The combined organic layers were dried over Na₂SO₄, then the solvent was removed under reduced pressure and the crude 5,5-dimethyl-4-oxo-piperidine-1,3-dicarboxylic acid 1-tert-butyl ester 3-methyl ester was used in the next step without further purification

Yield: 100%; LCMS (RT): 6.39 min (Method D); MS (ES+) gave m/z: 286.2 (MH+).

### 49(C) 1-(4-Fluoro-benzoyl)-5,5-dimethyl-4-oxo-piperidine-3-carboxylic Acid Methyl Ester

A solution of 5,5-dimethyl-4-oxo-piperidine-1,3-dicarboxylic acid-1-tert-butyl ester-3-methyl ester (200 mg, 0.70 mmol) in DCM (5 mL) was cooled at 0° C. HCl (4M in dioxane, 1.5 mL, 6 mmol) was added and the mixture was stirred at room temperature for 1 h. The solvent was removed under reduced pressure and the crude was dissolved in DCM (5 mL). Triethylamine (293 μL, 2.1 mmol) and 4-fluorobenzoyl chloride (99 μL, 0.84 mmol) were added and the mixture was stirred at room temperature for 2 h. The organic layer was washed with 1M HCl (2×5 mL), with NaHCO₃ (2×5 mL), then it was dried over Na₂SO₄. The solvent was removed under reduced pressure and the crude was purified by flash chromatography (silica gel, eluent: hexane/ethyl acetate 10:1) to yield 1-(4-fluoro-benzoyl)-5,5-dimethyl-4-oxo-piperidine-3-carboxylic acid methyl ester.

Yield: 21%; LCMS (RT): 5.28 min (Method D); MS (ES+) gave m/z: 308.16 (MH+).

### 49(D) 1-(4-Fluoro-benzoyl)-4-hydroxy-5,5-dimethyl-piperidine-3-carboxylic Acid Methyl Ester

To a solution of 1-(4-fluoro-benzoyl)-5,5-dimethyl-4-oxo-piperidine-3-carboxylic acid methyl ester (80 mg, 0.26 mmol) in MeOH (1 mL), NaBH₄ (10 mg, 0.26 mmol) was added. The mixture was stirred at room temperature for 15 min, then acetone (5 mL) was added. The solvent was removed under reduced pressure, the crude was dissolved in ethyl acetate and washed with 1M HCl (2×5 mL). The crude 1-(4-fluoro-benzoyl)-4-hydroxy-5,5-dimethyl-piperidine-3-carboxylic acid methyl ester was used in the next step without further purification.

Yield: 100%; LCMS (RT): 3.73 min (Method D); MS (ES+) gave m/z: 310.29 (MH+).

### 49(E) 1-(4-Fluoro-benzoyl)-5,5-dimethyl-1,2,5,6-tetrahydro-pyridine-3-carboxylic Acid Methyl Ester

A solution of 1-(4-fluoro-benzoyl)-4-hydroxy-5,5-dimethyl-piperidine-3-carboxylic acid methyl ester (280 mg, 0.91 mmol) in DCM (10 mL) was cooled at 0° C., then triethylamine (380 μL, 2.73 mmol) and MsCl (106 μL, 1.37 mmol) were added. The mixture was stirred at room temperature for 3 h, then the solution was washed with H₂O (2×10 mL) and dried over Na₂SO₄. The solvent was removed under reduced pressure and the crude was dissolved in toluene (5 mL). DBU (272 μL, 1.82 mmol) was added and the mixture was heated at 80° C. for 30 min. The solution was diluted with DCM and washed with 1M HCl (2×15 mL). The organic layer was dried over Na₂SO₄ then the solvent was removed under reduced pressure. The crude was purified by flash chromatography (silica gel, eluent: DCM/Methanol 100:1) to yield 1-(4-fluoro-benzoyl)-5,5-dimethyl-1,2,5,6-tetrahydro-pyridine-3-carboxylic acid methyl ester.

Yield: 48%; LCMS (RT): 4.86 min (Method D) MS (ES+) gave m/z: 292.24 (MH+).

### 49(F) 1-(4-Fluoro-benzoyl)-5,5-dimethyl-piperidine-3-carboxylic Acid Methyl Ester

To a suspension of 10% Pd/C (20 mg) in EtOH (10 mL), 1-(4-fluoro-benzoyl)-5,5-dimethyl-1,2,5,6-tetrahydro-pyridine-3-carboxylic acid methyl ester (125 mg, 0.43 mmol) was added. The mixture was hydrogenated (40 psi, room temperature) overnight. The mixture was then filtered over a celite pad, the solvent was removed under reduced pressure and the crude was purified by flash chromatography (silica gel, eluent: hexane/ethyl acetate 80:20) to yield 1-(4-fluoro-benzoyl)-5,5-dimethyl-piperidine-3-carboxylic acid methyl ester.

Yield: 37%; LCMS (RT): 4.88 min (Method D); MS (ES+) gave m/z: 294.25 (MH+).

### 49 (G) Lithium 1-(4-fluoro-benzoyl)-5,5-dimethyl-piperidine-3-carboxylate

To a solution of 1-(4-fluoro-benzoyl)-5,5-dimethyl-piperidine-3-carboxylic acid methyl ester (43 mg, 0.15 mmol) in THF/MeOH 1:1 (5 mL), LiOH (4 mg, 0.15 mmol) and H₂O (100 μL) were added. The mixture was stirred overnight at room temperature, then the solvent was removed under reduced pressure and the crude Lithium 1-(4-fluoro-benzoyl)-5,5-dimethyl-piperidine-3-carboxylate was used in the next step without further purification.

Yield: 100%; LCMS (RT): 4.02 min (Method D); MS (ES+) gave m/z: 280.26, (MH+).

### 49 (H) (3,3-Dimethyl-5-[3-(1H-pyrrol-2-yl)-[1,2,4]oxadiazol-5-yl]-piperidin-1-yl)-(4-fluoro-phenyl)-methanone

A mixture of lithium 1-(4-fluoro-benzoyl)-5,5-dimethyl-piperidine-3-carboxylate (42 mg, 0.15 mmol), HOAT (20 mg, 0.15 mmol) and EDCI.HCl (43 mg, 0.23 mmol) in dioxane (2 mL) was stirred at room temperature for 10 min. N-Hydroxy-1H-pyrrole-2-carboxamidine (19 mg, 0.15 mmol, prepared as described in Example 1(A)) and triethylamine (41 μL, 0.30 mmol) were added. The mixture was stirred for 3 days at room temperature, then at 80° C. for 4 h. The solvent was removed under reduced pressure then the crude was dissolved in DCM and washed with 5% Na₂CO₃ (aq) (2×5 mL). The organic layer was dried over Na₂SO₄ then the solvent was removed under reduced pressure and the crude was purified by flash chromatography (silica gel; eluent: DCM/methanol 98:2).

Yield: 60% (white solid); LCMS (RT): 3.09 min (Method N); MS (ES+) gave m/z: 369.2 (MH+).

¹H-NMR (DMSO-d₆ 353K), δ (ppm): 11.42 (s br, 1H); 7.50 (dd, 2H); 7.25 (dd, 2H); 6.96 (dd, 1H); 6.73 (dd, 1H); 6.21 (dd, 1H); 4.47 (m, 1H); 3.71 (m, 1H); 3.46 (m, 1H); 3.21-3.04 (m, 2H); 2.00 (m, 1H); 1.74 (dd, 1H); 0.99 (s, 3H); 0.96 (s, 3H).

### Example 50

### (4-Fluoro-phenyl)-{(S)-3-[3-(4-fluoro-1H-pyrrol-2-yl)-[1,2,4]oxadiazol-5-yl]-piperidin-1-yl}-methanone

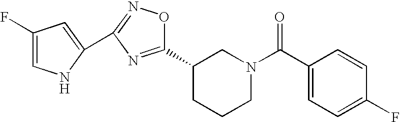

### 50(A) (S)-4-Oxo-N-Boc-pyrrolidine-2-carboxylic Acid Methyl Ester

A solution of DMSO (1.38 mL, 19.5 mmol) in dry DCM (30 mL) was cooled to −78° C. and oxalyl chloride (1.65 mL, 18 mmol) was added. After stirring at −78° C. under N₂ for 15 min, N-Boc-trans-4-hydroxy proline methyl ester (3.07 g, 12.5 mmol) was added and the resulting solution stirred for 4 hours at −50° C. under N₂. Triethylamine (5 mL, 36 mmol) was added, and the solution allowed to warm slowly to room temperature, then stirred overnight. The solution was diluted with approx 50 mL of DCM then washed twice with 10% citric acid aqueous solution, then with water and with brine. The solution was dried over sodium sulphate and the solvent removed to give the product as a pale yellow oil.

Yield: 100%; LCMS (RT): 3.53 min (Method A); MS (ES+) gave m/z: 244 (MH+).

### 50(B) (S)-4,4-Difluoro-N-Boc-pyrrolidine-2-carboxylic Acid Methyl Ester

A solution of (S)-4-oxo-N-Boc-pyrrolidine-2-carboxylic acid methyl ester (1 g, 4.1 mmol) in dry DCM (10 mL) was cooled to −78° C. under N₂, and then diethylamino sulfur trifluoride (1.95 mL, 16 mmol) was added. The mixture was stirred at −78° C. for 10 minutes then allowed to warm to room temperature and stirred under N₂ for 2 hours. Ice was added and the solution was then basified with 5% NaHCO₃ (aq) and extracted three times with DCM. The combined organic extracts were washed with 5% NaHCO₃ (aq) solution, water and brine, dried over sodium sulphate and the solvent removed to give the required product as a yellow oil.

Yield: 99%; LCMS (RT): 5.03 min (Method D); MS (ES+) gave m/z: 266 (MH+).

### 50(C) (S)-4,4-Difluoropyrrolidine-2-carboxylic Acid Methyl Ester Trifluoroacetate

(S)-4,4-Difluoro-N-Boc-pyrrolidine-2-carboxylic acid methyl ester (1.08 g, 4.07 mmol) was dissolved in TFA (5 mL) and stirred under N₂ for 30 min. The solvent was removed under vacuum and the residue dissolved in MeOH, loaded onto an SCX ion exchange column, washed with MeOH and DCM then eluted with 5% NH₃ in MeOH. The solvent was removed to give the product as a pale brown oil.

Yield: 77%; LCMS (RT): 0.63 min (Method A); MS (ES+) gave m/z: 166 (MH+).

### 50(D) (S)-4,4-Difluoro-N-tosyl-pyrrolidine-2-carboxylic Acid Methyl Ester

Tosyl chloride (667 mg, 3.5 mmol) and triethylamine (550 μL, 4 mmol) were added to a solution of (S)-4,4-difluoropyrrolidine-2-carboxylic acid methyl ester trifluoroacetate (520 mg, 3.15 mmol) in DCM and the resulting mixture was stirred for two days. The solution was washed twice with 10% citric acid solution, then with 5% NaHCO₃ solution and with brine, dried and the solvent removed. The residue was purified by flash chromatography (silica gel cartridge, eluent gradient: from hexane/ethyl acetate 100:0 to hexane/ethyl acetate 70:30) to give the product as a colourless oil which solidified on standing.

Yield: 76%; LCMS (RT): 5.2 min (Method D); MS (ES+) gave m/z: 320 (MH+).

### 50(E) 4-Fluoro-1H-pyrrole-2-carboxylic Acid Methyl Ester

Sodium (830 mg, 35 mmol) was dissolved in dry MeOH (10 mL) under N₂ and then added to a solution of (S)-4,4-difluoro-N-tosyl-pyrrolidine-2-carboxylic acid methyl ester (765 mg, 2.4 mmol) in dry MeOH (10 mL). The solution was stirred under N₂ for 2 hours and then the solvent was removed under vacuum. 10% Citric acid aqueous solution (30 mL) was added and the solution extracted three times with EtOAc. The combined organic extracts were dried over sodium sulphate and the solvent removed. The residue was purified by flash chromatography (silica gel cartridge, eluent gradient: from hexane/ethyl acetate 100:0 to hexane/ethyl acetate 75:25) to give the product as a white solid.

Yield: 77%; LCMS (RT): 3.7 min (Method D); MS (ES+) gave m/z: 112 [M-OMe]⁺

### 50(F) 4-Fluoro-1H-pyrrole-2-carboxylic Acid

4-Fluoro-1H-pyrrole-2-carboxylic acid methyl ester (264 mg, 1.85 mmol) and NaOH (75 mg, 1.9 mmol) were dissolved in 1:1 dioxane/water (10 mL) and stirred overnight. The solvent was removed, 10% citric acid aqueous solution (20 mL) added and the solution extracted three times with EtOAc. The combined organic extracts were washed with brine, dried over sodium sulphate and the solvent removed to give the product as a white solid.

Yield: 97% LCMS (RT): 2.7 min (Method D); MS (ES+) gave m/z: 130 (MH+).

### 50(G) 4-Fluoro-1H-pyrrole-2-carboxylic Acid Amide

Carbonyl diimidazole (340 mg, 2.1 mmol) was added to a solution of 4-fluoro-1H-pyrrole-2-carboxylic acid (230 mg, 1.78 mmol) in MeCN (10 mL) and stirred for 90 min. Concentrated NH₄OH solution (2 mL) was added and the resulting mixture refluxed for 90 min. The solvent was removed, 10% citric acid solution (10 mL) was added and the solution extracted three times with EtOAc. The organic extracts were combined, dried over sodium sulphate and the solvent removed to give the product as a white solid.

Yield: 100% LCMS (RT): 2.1 min (Method G); MS (ES+) gave m/z: 129 (MH+).

### 50(H) 4-Fluoro-1H-pyrrole-2-carbonitrile

A solution of 4-fluoro-1H-pyrrole-2-carboxylic acid amide (210 mg, 1.7 mmol) in phosphorus oxychloride (5 mL) was heated at 100° C. for 5 minutes, cooled, ice was added, basified with conc. NH₄OH solution then extracted three times with EtOAc. The organic extracts were combined, dried and the solvent removed to give the product as a pale brown oil

Yield: 90% LCMS (RT): 3.5 min (Method G); MS (ES+) gave m/z: 111 (MH+).

### 50(I) 4-Fluoro-N-hydroxy-1H-pyrrole-2-carboxamidine

50% Hydroxylamine solution in water (1.2 mL, 20 mmol) was added to a solution of 4-fluoro-1H-pyrrole-2-carbonitrile (176 mg, 1.6 mmol) in ethanol (3 mL) and heated under reflux for 1 h. The solvent was removed under vacuum and the residue purified by flash chromatography (silica gel cartridge, eluent gradient: from hexane/ethyl acetate 100:0 to hexane/ethyl acetate 0:100) to give the product as a white solid.

Yield: 95% LCMS (RT): 1.4 min (Method G); MS (ES+) gave m/z: 144 (MH+).

### 50(J) (S)-3-[3-(4-Fluoro-1H-pyrrol-2-yl)-[1,2,4]oxadiazol-5-yl]-piperidine-1-carboxylic Acid tert-butyl Ester

A mixture of (S)-N-Boc-nipecotic acid (229 mg, 1 mmol), HOAT (163 mg, 1.2 mmol), EDCI.HCl (230 mg, 1.2 mmol) in dry DCM (10 mL) was stirred under N₂ for 10 minutes, then 4-fluoro-N-hydroxy-1H-pyrrole-2-carboxamidine (131 mg, 0.92 mmol) was added and the solution stirred overnight. The solution was washed with water, 10% citric acid solution and 5% NaHCO₃ solution, dried over sodium sulphate and the solvent removed to give a residue that was purified by flash chromatography (silica gel cartridge, eluent gradient: from hexane/ethyl acetate 100:0 to hexane/ethyl acetate 80:20). The solid thus obtained was dissolved in acetonitrile (2 mL) and heated in a sealed tube at 75° C. for 90 min in a microwaves reactor. The solvent was removed and the crude residue was purified by flash chromatography (silica gel cartridge, eluent gradient: from hexane/ethyl acetate 100:0 to hexane/ethyl acetate 80:20) to give the product as a white solid.

Yield: 64%; LCMS (RT): 5.8 min (Method D); MS (ES+) gave m/z: 337 (MH+).

### 50(K) (S)-3-[3-(4-Fluoro-1H-pyrrol-2-yl)-[1,2,4]oxadiazol-5-yl]-piperidine Trifluoroacetate Salt

(S)-3-[3-(4-Fluoro-1H-pyrrol-2-yl)-[1,2,4]oxadiazol-5-yl]-piperidine-1-carboxylic acid tert-butyl ester (200 mg, 0.59 mmol) was dissolved in DCM (5 mL) and trifluoroacetic acid (2 mL) added. The solution was stirred for 30 min and then the solvent removed and dried under high vacuum.

Yield: 100%; LCMS (RT): 2.6 min (Method D); MS (ES+) gave m/z: 237 (MH+).

### 50(L) (4-Fluoro-phenyl)-{(S)-3-[3-(4-fluoro-1H-pyrrol-2-yl)-[1,2,4]oxadiazol-5-yl]-piperidin-1-yl}-methanone

(S)-3-[3-(4-Fluoro-1H-pyrrol-2-yl)-[1,2,4]oxadiazol-5-yl]-piperidine trifluoroacetate salt (104 mg, 0.3 mmol) was dissolved in DCM (5 mL) and 4-fluoro-benzoyl chloride (49 μL, 0.4 mmol) was added followed by triethylamine (125 μL, 0.9 mmol). The solution was stirred for 1 hour, then washed with 0.1 M HCl solution, with 0.1 M NaOH and the solvent removed. The residue was purified by flash chromatography (silica gel cartridge, eluent gradient: from hexane/ethyl acetate 100:0 to hexane/ethyl acetate 30:70) to give the product as a white solid.

Yield: 68%; [α]_{D}²⁰=+116.6 (c=0.5, MeOH); mp=146.5-147.2° C.; LCMS (RT): 2.84 min (Method N); MS (ES+) gave m/z: 359.1 (MH+).

¹H-NMR (DMSO-d₆ 353K), δ (ppm): 11.38 (s br, 1H); 7.46 (dd, 2H); 7.42 (dd, 2H); 6.83 (m, 1H); 6.53 (m, 1H); 4.22 (dd, 1H); 3.76 (dt, 1H); 3.50 (dd, 1H); 3.40-3.21 (m, 2H); 2.24 (m, 1H); 2.03-1.76 (m, 2H); 1.64 (m, 1H).

### Example 51

### (3,4-Difluoro-phenyl)-{(S)-3-[3-(4-fluoro-1H-pyrrol-2-yl)-[1,2,4]oxadiazol-5-yl]-piperidin-1-yl}-methanone

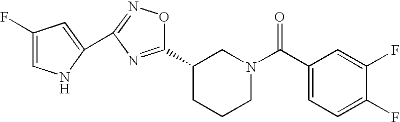

(S)-3-[3-(4-Fluoro-1H-pyrrol-2-yl)-[1,2,4]oxadiazol-5-yl]-piperidine trifluoroacetate salt (104 mg, 0.3 mmol) (prepared as described in example 50(K)) was dissolved in DCM (5 mL) and 3,4-difluorobenzoyl chloride (50 μL, 0.4 mmol) was added followed by triethylamine (125 μL, 0.9 mmol). The solution was stirred for 1 hour then washed with 0.1 M HCl solution, with 0.1 M NaOH and then the solvent was removed. The residue was purified by flash chromatography (silica gel cartridge, eluent gradient: from hexane/ethyl acetate 100:0 to hexane/ethyl acetate 30:70) to give the product as a white solid.

Yield: 63%; [α]_{D}²⁰=+111.2 (c=0.5, MeOH); mp=147.5-148.2° C.; LCMS (RT): 2.91 min (Method N); MS (ES+) gave m/z: 377.0 (MH+).

¹H-NMR (DMSO-d₆ 353K), δ (ppm): 11.39 (s br, 1H); 7.50-7.39 (m, 2H); 7.25 (m, 1H); 6.84 (m, 1H); 6.53 (m, 1H); 4.20 (dd, 1H); 3.74 (dt, 1H); 3.51 (dd, 1H); 3.42-3.23 (m, 2H); 2.23 (m, 1H); 2.02-1.75 (m, 2H); 1.65 (m, 1H).

### Example 52

### (6-Fluoro-pyridin-3-yl)-{(S)-3-[3-(4-fluoro-1H-pyrrol-2-yl)-[1,2,4]oxadiazol-5-yl]-piperidin-1-yl}-methanone

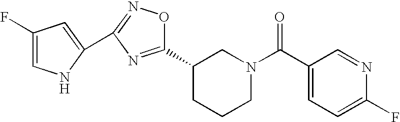

### 52(A) (S)-3-[3-(4-Fluoro-1H-pyrrol-2-yl)-[1,2,4]oxadiazol-5-yl]-piperidine Hydrochloride Salt

(S)-3-[3-(4-Fluoro-1H-pyrrol-2-yl)-[1,2,4]oxadiazol-5-yl]-piperidine-1-carboxylic acid tert-butyl ester (120 mg, 0.36 mmol) (prepared as described in example 50(J)) was dissolved in DCM (1 mL) and 4M HCl in dioxane (2 mL) added. The solution was stirred for 30 min at room temperature and then the solvent removed and dried under high vacuum.

Yield: 100%; LCMS (RT): 2.6 min (Method D); MS (ES+) gave m/z: 237 (MH+).

### 52(B) (6-Fluoro-pyridin-3-yl)-{(S)-3-[3-(4-fluoro-1H-pyrrol-2-yl)-[1,2,4]oxadiazol-5-yl]-piperidin-1-yl}-methanone

A mixture of 6-fluoro nicotinic acid (56 mg, 0.4 mmol), HOAT (68 mg, 0.5 mmol), EDCI.HCl (96 mg, 0.5 mmol) in dry DCM (10 mL) was stirred under N₂ for 10 minutes at room temperature, then (S)-3-[3-(4-fluoro-1H-pyrrol-2-yl)-[1,2,4]oxadiazol-5-yl]-piperidine hydrochloride salt (98 mg, 0.36 mmol) and triethylamine (83 μL, 0.6 mmol) were added and the solution stirred for 1 hour at room temperature. The solution was washed with water and with 0.2M NaOH solution, dried and the solvent removed to give a residue that was purified by flash chromatography (silica gel cartridge, eluent gradient: from hexane/ethyl acetate 100:0 to hexane/ethyl acetate 30:70) to give the product as a colourless gum.

Yield: 77%; [α]_{D}²⁰=+72 (c=0.3, MeOH); LCMS (RT): 3.27 min (Method P); MS (ES+) gave m/z: 360.1 (MH+).

¹H-NMR (DMSO-d₆ 353K), δ (ppm): 11.45 (s br, 1H); 8.31 (m, 1H); 8.02 (ddd, 1H); 7.22 (dd, 1H); 6.85 (dd, 1H); 6.54 (d, 1H); 4.23 (m, 1H); 3.77 (m, 1H); 3.55 (dd, 1H); 3.46-3.26 (m, 2H); 2.23 (m, 1H); 2.04-1.75 (m, 2H); 1.67 (m, 1H).

### Example 53

### (2-Fluoro-pyridin-4-yl)-{(S)-3-[3-(4-fluoro-1H-pyrrol-2-yl)-[1,2,4]oxadiazol-5-yl]-piperidin-1-yl}-methanone

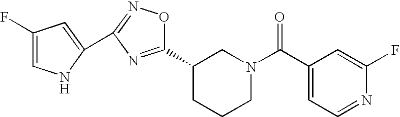

A mixture of 2-fluoro isonicotinic acid (42 mg, 0.3 mmol), HOAT (41 mg, 0.3 mmol), EDCI.HCl (58 mg, 0.3 mmol) in dry DCM (10 mL) was stirred at room temperature under N₂ for 10 minutes, then (S)-3-[3-(4-fluoro-1H-pyrrol-2-yl)-[1,2,4]oxadiazol-5-yl]-piperidine hydrochloride salt (63 mg, 0.23 mmol) (prepared as described in example 52(A)) and triethylamine (83 μL, 0.6 mmol) were added and the solution stirred overnight at room temperature. The solution was washed with water and with 0.2M NaOH solution, dried and the solvent removed to give a residue that was purified by flash chromatography (silica gel cartridge, eluent gradient: from hexane/ethyl acetate 100:0 to hexane/ethyl acetate 0:100) to give the product as a colourless gum.

Yield: 73%; [α]_{D}²⁰=+110 (c=0.7, MeOH); LCMS (RT): 2.50 min (Method N); MS (ES+) gave m/z: 360.3 (MH+).

¹H-NMR (DMSO-d₆ 353K), δ (ppm): 11.44 (s br, 1H); 8.32 (d, 1H); 7.33 (ddd, 1H); 7.15 (m, 1H); 6.86 (dd, 1H); 6.54 (d, 1H); 4.18 (m, 1H); 3.71 (m, 1H); 3.53 (dd, 1H); 3.45-3.22 (m, 2H); 2.22 (m, 1H); 2.04-1.75 (m, 2H); 1.67 (m, 1H).

### Example 54

### (4-Fluoro-phenyl)-{(S)-3-[5-(1H-pyrrol-2-yl)-tetrazol-2-yl]-piperidin-1-yl}-methanone

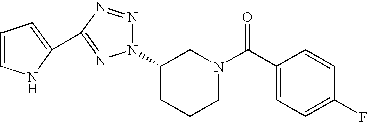

### 54(A) (4-Fluoro-phenyl)-((R)-3-hydroxy-piperidin-1-yl)-methanone

A mixture of (R)-3-hydroxy piperidine hydrochloride (0.2 g, 1.45 mmol), 4-fluoro benzoic acid (0.204 g, 1.45 mmol), EDC.HCl (0.42 g, 2.18 mmol), HOBT (0.196 g, 1.45 mmol), triethylamine (320 μL, 4.36 mmol) in dichloromethane (10 mL) was stirred under nitrogen atmosphere overnight at room temperature. The reaction mixture was diluted with dichloromethane (20 mL) and washed subsequently with 0.1N HCl (2 times), with 0.1N NaOH (2 times) and then with brine. The organic layer was dried over sodium sulphate and evaporated under reduced pressure to give a pale yellow oil (275 mg), which was used for the next step without further purification.

Yield: 85%; [α]_{D}²⁰=−8.7 (c=0.615, CHCl₃); LCMS (RT): 3.1 min (Method D); MS (ES+) gave m/z: 224.0 (MH+).

¹H-NMR (CDCl₃); 5 (ppm): 7.43 (dd, 2H); 7.08 (dd, 2H); 4.00-3.14 (m br, 5H); 2.27 (s br, 1H); 1.98-1.76 (m, 2H); 1.74-1.55 (m, 2H).

### 54(B) 5-(1H-Pyrrol-2-yl)-2H-tetrazole

2-Cyanopyrrole (300 μL, 3.55 mmol), sodium azide (275 mg, 4.25 mmol) and ammonium chloride (134 mg, 4.25 mmol) were dissolved in DMF (1 mL) and heated in a sealed tube in a microwave reactor for 20 min at 120° C., then for 25 min at 160° C. and then for 5 min at 180° C. After cooling, the tube was vented to release the pressure generated during the reaction and water was added. The solution was washed with EtOAc, acidified to about pH 3 with 1M HCl and then extracted three times with DCM. The combined organic extracts were dried and the solvent removed to give the product as a white solid.

Yield: 57%; LCMS (RT): 1.8 min (Method D); MS (ES+) gave m/z: 136 (MH+).

¹H-NMR (DMSO); δ (ppm): 11.92 (s br, 1H); 7.01 (d, 1H); 6.79 (d, 1H); 6.24 (dd, 1H).

### 54(C) (4-Fluoro-phenyl)-{(S)-3-[5-(1H-pyrrol-2-yl)-tetrazol-2-yl]-piperidin-1-yl}-methanone

Diisopropylazadicarboxylate (DIAD, 141 μL, 0.72 mmol) was added dropwise at 0° C. with stirring to a mixture of 5-(1H-pyrrol-2-yl)-2H-tetrazole (95 mg, 0.7 mmol), (4-fluoro-phenyl)-((R)-3-hydroxy-piperidin-1-yl)-methanone (100 mg, 0.36 mmol) and solid supported triphenylphosphine (PS-PPh₃, ex Argonaut Technologies, loading 2.4 mmol/g, 420 mg, 1 mmol) in dichloromethane (4 mL). The mixture was heated in a sealed tube in a microwave reactor at 100° C. for 30 min. The resin was filtered off and washed with DCM and MeOH. The combined solutions were concentrated under vacuum and the residue purified by flash chromatography (silica gel cartridge, eluent gradient: from DCM/MeOH 100:0 to DCM/MeOH 98:2) The crude material thus recovered was then dissolved in toluene and passed through a silica gel cartridge (Isolute Flash II 2 g, eluted with hexane, then with hexane/diethyl ether 75:25, then with hexane/diethyl ether 60:40, then with DCM/MeOH 98:2).

The title compound was obtained pure as a colourless gum.

Yield: 30%; LCMS (RT): 6.28 min (Method Q); MS (ES+) gave m/z: 341.2 (MH+).

¹H-NMR (DMSO-d₆ 368K), δ ppm): 11.31 (s br, 1H); 7.45 (dd, 2H); 7.19 (dd, 2H); 6.93 (m, 1H); 6.70 (m, 1H); 6.21 (m, 1H); 4.99 (dddd, 1H); 4.31 (dd, 1H); 3.77 (dd, 1H); 3.71 (m, 1H); 3.42 (ddd, 1H); 2.47-2.23 (m, 2H); 2.03-1.90 (m, 1H); 1.73 (m, 1H).

### Example 55

### (4-Fluoro-phenyl)-{(S)-3-[5-(4-trifluoromethyl-1H-imidazol-2-yl)-[1,2,4]oxadiazol-3-yl]-piperidin-1-yl}-methanone

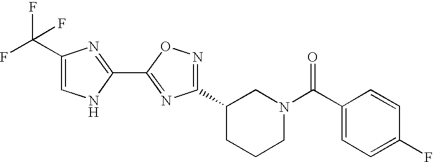

### 55(A) 4-Trifluoromethyl-1H-imidazole-2-carboxylic Acid Ethyl Ester

3,3-Dibromo-1,1,1-trifluoropropanone (1 g, 3.7 mmol) was added to a solution of sodium acetate trihydrate (1 g, 7.4 mmol) in water (5 mL) and the mixture refluxed for 30 min. After cooling, a solution of ethyl glyoxalate (590 μL, 3 mmol) and conc. ammonia solution (500 mL) in MeOH (2 mL) was added and the mixture stirred for 24 hours at room temperature. The pH was adjusted to about 8 and the solution extracted three times with EtOAc. The combined organic extracts were dried and the solvent removed to give the product as a white solid.

Yield: 69%; LCMS (RT): 3.31 min (Method A); MS (ES+) gave m/z: 209 (MH+).

### 55(B) 4-Trifluoromethyl-1H-imidazole-2-carboxylic Acid Sodium Salt

4-Trifluoromethyl-1H-imidazole-2-carboxylic acid ethyl ester (245 mg, 1.18 mmol) was dissolved in 5M NaOH solution (235 μL, 1.18 mmol) and heated for 12 hours at 70° C. The solvent was removed by azeotropic distillation with toluene to give the product as a white solid.

Yield: 100%; LCMS (RT): 2.32 min (Method D); MS (ES+) gave m/z: 181 (MH+).

### 55(C) (S)-3-[5-(4-Trifluoromethyl-1H-imidazol-2-yl)-[1,2,4]oxadiazol-3-yl]-piperidine-1-carboxylic Acid tert-butyl Ester

4-Trifluoromethyl-1H-imidazole-2-carboxylic acid (417 mg, 2.06 mmol) and (S)-3-(N-hydroxycarbamimidoyl)-piperidine-1-carboxylic acid tert-butyl ester (500 mg, 2.06 mmol) (prepared as described in Example 10(C)), were dissolved in dioxane (5 mL). HOAt (561 mg, 4.12 mmol) was added with stirring, followed by EDC.HCl (593 mg, 3.1 mmol). The solution was heated at 70° C. for 9 h, cooled, water was added and the solution was extracted three times with EtOAc. The combined organic extracts were dried and the solvent removed. The solid thus obtained was dissolved in acetonitrile (2 mL) and heated in a sealed tube at 80° C. for 1 hour in a microwave reactor. The solvent was removed, the residue dissolved in EtOAc and washed twice with 5% citric acid solution, with 1M NaOH and with brine and the solvent removed. The residue was purified by flash chromatography (Biotage silica gel, eluted with EtOAc/hexane 10:90) to give the required product.

Yield: 10%; LCMS (RT): 4.18 min (Method A); MS (ES+) gave m/z: 389 (MH+).

### 55(D) (S)-3-[5-(4-Trifluoromethyl-1H-imidazol-2-yl)-[1,2,4]oxadiazol-3-yl]-piperidine Hydrochloride Salt

(S)-3-[5-(4-Trifluoromethyl-1H-imidazol-2-yl)-[1,2,4]oxadiazol-3-yl]-piperidine-1-arboxylic acid tert-butyl ester (83 mg, 0.214 mmol) was dissolved in a 2:1 mixture of DCM/MeOH (3 mL) and 4M HCl in dioxane (1 mL) was added at 0° C. The solution was stirred under N₂ for 2 hours at room temperature, then the solvent was removed to give the product as a white solid.

Yield: 100%; LCMS (RT): 2.80 min (Method A); MS (ES+) gave m/z: 289 (MH+).

### 55(E) (4-Fluoro-phenyl)-{(S)-3-[5-(4-trifluoromethyl-1H-imidazol-2-yl)-[1,2,4]oxadiazol-3-yl]-piperidin-1-yl}-methanone

(S)-3-[5-(4-Trifluoromethyl-1H-imidazol-2-yl)-[1,2,4]oxadiazol-3-yl]-piperidine hydrochloride salt (70 mg, 0.214 mmol) was suspended in dry DCM (7 mL) at 0° C. and triethylamine (63 μL, 0.45 mmol) added, followed by 4-fluorobenzoyl chloride (25 μL, 0.214 mmol). The mixture was stirred under N₂ at room temperature for 3 hours then washed with water, 5% citric acid solution and brine, dried and the solvent removed. The residue was purified by preparative HPLC to give the title compound.

Yield: 13%; LCMS (RT): 2.76 min (Method N); MS (ES+) gave m/z: 410.1 (MH+).

¹H-NMR (DMSO-d₆ 353K), δ (ppm): 7.97 (m, 1H); 7.47 (dd, 2H); 7.21 (dd, 2H); 4.28 (m, 1H); 3.83 (m, 1H); 3.38 (dd, 1H); 3.29-3.12 (m, 2H); 2.24 (m, 1H); 2.00-1.76 (m, 2H); 1.65 (m, 1H).

### Example 56

### (6-Fluoro-pyridin-3-yl)-{(S)-3-[5-(4-isopropyl-1H-pyrrol-2-yl)-[1,2,4]oxadiazol-3-yl]-piperidin-1-yl}-methanone

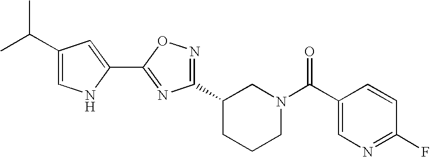

### 56(A) 3-Methyl-2-methylene-butyraldehyde

The compound was prepared as described in Tetrahedron, 1996, 1231-1234.

Yield: 37%; ¹H-NMR (CDCl₃): 9.54 (s, 1H), 6.23 (d, 1H), 5.94 (s, 1H), 2.81 (m, 1H), 1.09 (d, 1H).

### 56 (B) (Toluene-4-sulfonylamino)-acetic Acid Methyl Ester

To a solution of (toluene-4-sulfonylamino)-acetic acid (2 g, 8.72 mmol) in methanol (60 mL), conc. H₂SO₄ (1.5 mL) was added. The mixture was stirred at room temperature for 3 h then the solvent was removed under reduced pressure. The crude was dissolved in DCM (20 mL) and the organic phase was washed with H₂O (1×20 mL), 5% Na₂CO₃ (aq) (1×20 mL) and brine (1×20 mL). The organic layer was dried over Na₂SO₄ and the solvent was removed under reduced pressure. The crude (toluene-4-sulfonylamino)-acetic acid methyl ester was used in the next step without further purification.

Yield: 98%; LCMS (RT): 3.47 min (Method A); MS (ES+) gave m/z: 244.03 (MH+).

### 56(C) 3-Hydroxy-4-isopropyl-1-(toluene-4-sulfonyl)-pyrrolidine-2-carboxylic Acid Methyl Ester

To a solution of 3-methyl-2-methylene-butyraldehyde (850 mg, 8.72 mmol) and (toluene-4-sulfonylamino)-acetic acid methyl ester (2.09 g, 8.59 mmol) in THF (60 mL), DBU (2.90 mL, 19.18 mmol) was added. The mixture was stirred overnight at room temperature, then the solvent was removed under reduced pressure and the crude was dissolved in diethyl ether (50 mL). The organic layer was washed with 1N HCl (1×50 mL), 5% NaHCO₃ (aq) (1×50 mL) and H₂O (1×50 mL), then it was dried over Na₂SO₄ and the solvent was removed under reduced pressure. The crude 3-hydroxy-4-isopropyl-1-(toluene-4-sulfonyl)-pyrrolidine-2-carboxylic acid methyl ester was used in the next step without further purification.

Yield: 99%; LCMS (RT): 3.94 min (Method A); MS (ES+) gave m/z: 341.00 (MH+).

### 56(D) 4-Isopropyl-1-(toluene-4-sulfonyl)-4,5-dihydro-1H-pyrrole-2-carboxylic Acid Methyl Ester

A solution of 3-hydroxy-4-isopropyl-1-(toluene-4-sulfonyl)-pyrrolidine-2-carboxylic acid methyl ester (2.89 g, 8.46 mmol) in pyridine (30 mL) was cooled at 0° C. POCl₃ (2 mL) was added dropwise over 5 min and the mixture was stirred at room temperature for 3 days. The mixture was poured into ice and diluted with diethylether. The two layers were separated and the organic phase was washed with HCl 5% (2×20 mL), 5% NaHCO₃ (aq) (2×20 mL) and brine (1×20 mL). The organic layer was dried over Na₂SO₄ then the solvent was removed under reduced pressure to yield the crude 4-isopropyl-1-(toluene-4-sulfonyl)-4,5-dihydro-1H-pyrrole-2-carboxylic acid methyl ester, that was used in the next step without further purification.

Yield: 68%; LCMS (RT): 4.35 min (Method A); MS (ES+) gave m/z: 324.03 (MH+).

### 56(E) 4-Isopropyl-1H-pyrrole-2-carboxylic Acid Methyl Ester

To a solution of 4-isopropyl-1-(toluene-4-sulfonyl)-4,5-dihydro-1H-pyrrole-2-carboxylic acid (1.86 g, 5.75 mmol) in toluene (100 mL), DBU (1.72 mL, 11.50 mmol) was added. The mixture was refluxed for 4 h, then was cooled to room temperature and diluted with diethyl ether. The organic layer was washed with 10% HCl (2×100 mL), 5% NaHCO₃ (aq) (2×100 mL) and brine (1×100 mL), then it was dried over Na₂SO₄ and the solvent was removed under reduced pressure to yield the crude 4-isopropyl-1H-pyrrole-2-carboxylic acid methyl ester that was used in the next step without further purification.

Yield: 65%; LCMS (RT): 3.94 min (Method A); MS (ES+) gave m/z: 168.05 (MH+).

### 56(F) 4-Isopropyl-1H-pyrrole-2-carboxylic Acid

A mixture of 4-isopropyl-1H-pyrrole-2-carboxylic acid methyl ester (530 mg, 3.17 mmol) and NaOH (400 mg, 9.51 mmol) in dioxane/H₂O 10/1 (110 mL) was refluxed for 4 h, then stirred at room temperature overnight. The solvent was removed under reduced pressure. The crude residue was dissolved in H₂O, then 5% HCl was added to adjust the pH to 2. The aqueous phase was extracted with AcOEt (3×30 mL), then the combined organic layers were dried over Na₂SO₄ and the solvent was removed under reduced pressure. 4-isopropyl-1H-pyrrole-2-carboxylic acid was used in the next step without further purification.

Yield: 97%; LCMS (RT): 1.16 min (Method H); MS (ES+) gave m/z: 154.14 (MH+).

### 56 (G) (S)-3-[5-(4-Isopropyl-1H-pyrrol-2-yl)-[1,2,4]oxadiazol-3-yl]-piperidine-1-carboxylic Acid tert-butyl Ester

A mixture of 4-isopropyl-1H-pyrrole-2-carboxylic acid (200 mg, 1.31 mmol), HOAT (180 mg, 1.31 mmol), EDCI.HCl (380 mg, 1.96 mmol) in dioxane (30 mL) was stirred at 50° C. for 2 h, then (S)-3-(N-hydroxycarbamimidoyl)-piperidine-1-carboxylic acid tert-butyl ester (320 mg, 1.31 mmol, prepared as described in Example 10 (C)) was added. The mixture was stirred overnight at 80° C., then at room temperature for 24 h. The solvent was removed under reduced pressure, the crude was dissolved in ethyl acetate and the organic layer was washed with 5% Na₂CO₃ (aq) (2×30 mL) and with brine (1×30 mL). The organic phase was dried over Na₂SO₄ and the solvent was removed under reduced pressure. The crude was dissolved in CH₃CN, triethylamine (182 μL, 1.3 mmol) was added and the mixture was heated at 130° C. for 5 h, in a sealed tube, in microwaves oven. The solvent was removed and the crude was purified through a silica gel cartridge (eluent: hexane/ethyl acetate 80:20) to yield (S)-3-[5-(4-isopropyl-1H-pyrrol-2-yl)-[1,2,4]oxadiazol-3-yl]-piperidine-1-carboxylic acid tert-butyl ester.

Yield: 100%; LCMS (RT): 4.72 min (Method A); MS (ES+) gave m/z: 261.14 (MH+).

### 56 (H) (6-Fluoro-pyridin-3-yl)-{(S)-3-[5-(4-isopropyl-1H-pyrrol-2-yl)-[1,2,4]oxadiazol-3-yl]-piperidin-1-yl}-methanone

A solution of (S)-3-[5-(4-isopropyl-1H-pyrrol-2-yl)-[1,2,4]oxadiazol-3-yl]-piperidine-1-carboxylic acid tert-butyl ester (500 mg, 1.31 mmol) in DCM (60 mL) was cooled at 0° C., then HCl (4M in dioxane, 2 mL, 8 mmol) was added. The mixture was stirred at room temperature for 15 h then the solvent was removed under reduced pressure. The crude was dissolved in DCM (50 mL), then 6-fluoro-nicotinic acid (185 mg, 1.31 mmol), HOAT (180 mg, 1.31 mmol), EDCI.HCl (380 mg, 1.96 mmol) and triethylamine (580 μL, 3.93 mmol) were added. The mixture was stirred for 3 days at room temperature then the solvent was removed under reduced pressure. The crude was dissolved in ethyl acetate and the organic layer was washed with 5% Na₂CO₃ (aq) (2×20 mL) and brine (1×20 mL). The organic phase was dried over Na₂SO₄ and the solvent was removed under reduced pressure. The crude was purified by flash chromatography (silica gel, eluent: hexane/ethyl acetate 50:50) to yield (6-fluoro-pyridin-3-yl)-{(S)-3-[5-(4-isopropyl-1H-pyrrol-2-yl)-[1,2,4]oxadiazol-3-yl]-piperidin-1-yl}-methanone.

Yield: 26% (brown oil); [α_{D}]=+90.8 (c=0.93, CH₃OH); LCMS (RT): 4.23 min (Method N); MS (ES+) gave m/z: 384.1 (MH+).

¹H-NMR (DMSO-d₆ 353K), δ (ppm): 11.71 (s br, 1H); 8.30 (m, 1H); 8.02 (ddd, 1H); 7.20 (dd, 1H); 6.92 (m, 1H); 6.84 (m, 1H); 4.23 (m, 1H); 3.81 (m, 1H); 3.38 (dd, 1H); 3.27 (ddd, 1H); 3.16-3.06 (m, 1H); 2.84 (sept, 1H); 2.19 (m, 1H); 1.97-1.75 (m, 2H); 1.66 (m, 1H); 1.21 (d, 6H).

### Example 57

### (4-Fluoro-phenyl)-{3-[3-(1H-pyrrol-2-yl)-[1,2,4]oxadiazol-5-yl]-pyrrolidin-1-yl}-methanone

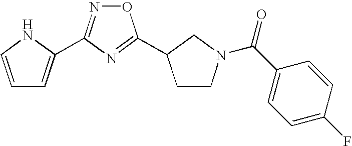

### 57(A) 3-[3-(1H-Pyrrol-2-yl)-[1,2,4]oxadiazol-5-yl]-pyrrolidine-1-carboxylic Acid tert-butyl Ester

To a solution of 1H-pyrrole-2-carbonitrile (0.110 mL, 1.3 mmol) in EtOH (2 mL), hydroxylamine (50% wt. aqueous solution, 0.318 mL, 5.2 mmol) was added at room temperature and the solution was stirred under reflux for 2 hours. The solvent was removed under reduced pressure to afford N-hydroxy-1H-pyrrole-2-carboxamidine that was used immediately for the next step.

A mixture of N-hydroxy-1H-pyrrole-2-carboxamidine (290 mg, 2.32 mmol), Boc-1-pyrrolidine-3-carboxylic acid (0.5 g, 2.32 mmol), EDCI.HCl (0.668 g, 3.48 mmol) and HOBT (0.358 g, 2.32 mmol) and triethylamine (977 μL, 6.96 mmol) in dioxane (40 mL) was stirred for 9 h under reflux, under nitrogen atmosphere. The solvent was evaporated under reduced pressure. The residue was diluted with water (20 mL) and ethyl acetate (20 mL), the phases were separated and the organic layer was washed sequentially with water (20 mL×2 times) and with 1N NaOH (20 mL×2 times), then with 5% citric acid solution. The organic layer was dried over Na₂SO₄ and concentrated under reduced pressure. 647 mg of 3-[3-(1H-pyrrol-2-yl)-[1,2,4]oxadiazol-5-yl]-pyrrolidine-1-carboxylic acid tert-butyl ester were obtained.

Yield: 92%; LCMS (RT): 7.8 min (Method F); MS (ES+) gave m/z: 305.3 (MH+).

### 57(B) 5-Pyrrolidin-3-yl-3-(1H-pyrrol-2-yl)-[1,2,4]oxadiazole Hydrochloride

3-[3-(1H-Pyrrol-2-yl)-[1,2,4]oxadiazol-5-yl]-pyrrolidine-1-carboxylic acid tert-butyl ester (0.64 g, 2.10 mmol) was dissolved in DCM (8 mL) and MeOH (0.5 mL) and 8 mL of 4N HCl (dioxane solution) were added dropwise at 0° C. The resulting mixture was stirred at room temperature for 4 h. The solvent was evaporated under reduced pressure to afford 497 mg (yield: 98%) of 5-pyrrolidin-3-yl-3-(1H-pyrrol-2-yl)-[1,2,4]oxadiazole hydrochloride as a white solid.

Yield: 98%; LCMS (RT): 2.33 min (Method F); MS (ES+) gave m/z: 205.3 (MH+).

### 57(C) (4-Fluoro-phenyl)-{3-[3-(1H-pyrrol-2-yl)-[1,2,4]oxadiazol-5-yl]-pyrrolidin-1-yl}-methanone

To a suspension of 5-pyrrolidin-3-yl-3-(1H-pyrrol-2-yl)-[1,2,4]oxadiazole hydrochloride (500 mg, 2.08 mmol) in dry dichloromethane (20 mL), triethylamine (0.614 mL, 4.37 mmol) and 4-fluorobenzoyl chloride (0.246 mL, 2.08 mmol) were added dropwise at 0° C. The reaction mixture was allowed to warm at room temperature and stirred under nitrogen atmosphere overnight. The solution was then treated with 1N NaOH (10 mL) and the phases were separated. The organic layer was washed with water (5 mL) and with brine (5 mL), then was dried over Na₂SO₄ and evaporated under reduced pressure. The crude was purified by flash chromatography (silica gel, eluent gradient: from petroleum ether/ethyl acetate 6:4 to petroleum ether/ethyl acetate 1:1) to give 213 mg of the title compound.

Yield: 33% (beige gummy solid); LCMS (RT): 5.56 min (Method R); MS (ES+) gave m/z: 327.2 (MH+).

¹H-NMR (DMSO-d₆ 353K), δ (ppm): 11.46 (s br, 1H); 7.60 (dd, 2H); 7.23 (dd, 2H); 6.97 (m, 1H); 6.75 (m, 1H); 6.22 (dd, 1H); 4.01-3.79 (m, 3H); 3.71-3.57 (m, 2H); 2.44 (m, 1H); 2.29 (m, 1H).

### Example 58

### (3-Fluoro-pyridin-4-yl)-{(S)-3-[5-(4-methyl-1H-pyrrol-2-yl)-[1,2,4]oxadiazol-3-yl]-piperidin-1-yl}-methanone

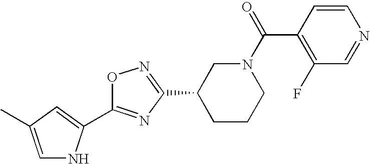

The title compound was prepared following the experimental procedure described in Example 28(C), starting from (S)-3-[5-(4-methyl-1H-pyrrol-2-yl)-[1,2,4]oxadiazol-3-yl]-piperidine hydrochloride, prepared as described in Example 28 (B), and using 3-fluoro-pyridine-4-carboxylic acid as the acid of choice.

Purification was performed by flash chromatography (silica gel, eluent: petroleum ether/ethyl acetate 1:2).

Yield: 98% (White amorphous solid); [α]_{D}²⁰=+101.8 (c=0.94; MeOH); LCMS (RT): 1.91 min (Method S); MS (ES+) gave m/z: 356.1 (MH+).

¹H-NMR (DMSO-d₆ 373 K), δ (ppm): 11.57 (s br 1H); 8.61 (s 1H); 8.50 (dd 1H); 7.43 (dd 1H); 6.89 (s 1H); 6.67 (s 1H); 4.45 (m br 1H); 3.95 (m br 1H); 3.38 (m 1H); 3.30 (m 1H); 3.06 (m 1H); 2.20 (m 1H); 2.11 (s 3H); 1.99-1.79 (m 2H); 1.63 (m 1H).

### Example 59

### {(S)-3-[5-(4-Chloro-1H-pyrrol-2-yl)-[1,2,4]oxadiazol-3-yl]-piperidin-1-yl}-(3-fluoro-pyridin-4-yl)-methanone

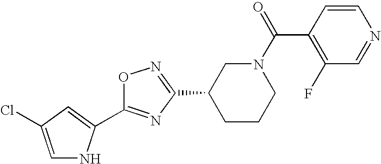

The title compound was prepared following the experimental procedure described in Example 28(C), starting from (S)-3-[5-(4-chloro-1H-pyrrol-2-yl)-[1,2,4]oxadiazol-3-yl]-piperidine hydrochloride, prepared as described in Example 39 (C), and using 3-fluoro-pyridine-4-carboxylic acid as the acid of choice.

Purification was performed by flash chromatography (silica gel, eluent: petroleum ether/ethyl acetate 2:8) and then by preparative HPLC.

Yield: 18%; LCMS (RT): 2.01 min (Method S); MS (ES+) gave m/z: 376.1 (MH+).

¹H-NMR (DMSO-d₆ 373K), δ (ppm): 12.26 (s br 1H); 8.61 (s 1H); 8.50 (d 1H); 7.43 (dd 1H); 7.18 (d 1H); 6.93 (s 1H); 4.51 (m br 1H); 3.87 (m br 1H); 3.46 (m 1H); 3.27 (m 1H); 3.10 (m 1H); 2.21 (m 1H); 2.00-1.80 (m 2H); 1.64 (m 1H).

### Example 60

### (2-Fluoro-pyridin-4-yl)-{(s)-3-[5-(4-fluoro-1H-pyrrol-2-yl)-[1,2,4]oxadiazol-3-yl]-piperidin-1-yl}-methanone

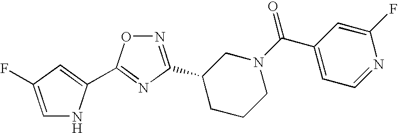

### 60(A) (S)-3-[5-(4-Fluoro-1H-pyrrol-2-yl)-[1,2,4]oxadiazol-3-yl]-piperidine-1-carboxylic Acid tert-butyl Ester

A mixture of 4-fluoro-1H-pyrrole-2-carboxylic acid (300 mg, 2.33 mmol), prepared as described in Example 50(F), (S)-3-(N-hydroxycarbamimidoyl)-piperidine-1-carboxylic acid tert-butyl ester (567 mg, 2.33 mmol), prepared as described in Example 10 (C), EDCI.HCl (672 mg, 3.5 mmol) and HOBT (315 mg, 2.33 mmol) in dioxane (10 mL) was stirred at room temperature overnight, then at 80° C. for 24 h, in the presence of activated 3 A molecular sieves. Molecular sieves were filtered off, then solvent was removed. Purification of the crude was performed by flash chromatography (silica gel, eluent: petroleum ether/ethyl acetate 8:2).

Yield: 38%; LCMS (RT): 5.91 min (Method D); MS (ES+) gave m/z: 337.0 (MH+).

### 60(B) (S)-3-[5-(4-Fluoro-1H-pyrrol-2-yl)-[1,2,4]oxadiazol-3-yl]-piperidine Hydrochloride

A solution of (S)-3-[5-(4-fluoro-1H-pyrrol-2-yl)-[1,2,4]oxadiazol-3-yl]-piperidine-1-carboxylic acid tert-butyl ester (77 mg, 0.23 mmol) in DCM (3 mL) was cooled at 0° C., then 4M HCl in dioxane (1 mL) was added. The mixture was stirred at room temperature for 2 h then the solvent was removed under reduced pressure.

Yield: quantitative.

### 60(C) (2-Fluoro-pyridin-4-yl)-{(s)-3-[5-(4-fluoro-1h-pyrrol-2-yl)-[1,2,4]oxadiazol-3-yl]-piperidin-1-yl}-methanone

The title compound was prepared following the experimental procedure described in Example 28(C), starting from (S)-3-[5-(4-fluoro-1H-pyrrol-2-yl)-[1,2,4]oxadiazol-3-yl]-piperidine hydrochloride, prepared as described in Example 60 (B), and using 2-fluoro-pyridine-4-carboxylic acid as the acid of choice.

Purification was performed by flash chromatography (silica gel, eluent: petroleum ether/ethyl acetate 4:6).

Yield: 61% (white solid); LCMS (RT): 1.97 min (Method S); MS (ES+) gave m/z: 360.0 (MH+).

¹H-NMR (DMSO-d₆ 353K), δ (ppm): 11.97 (s br 1H); 8.32 (d 1H); 7.34 (m 1H); 7:16 (m 1H); 7.04 (dd 1H); 6.78 (m 1H); 4.24 (m br 1H); 3.76 (m br 1H); 3.46-3.05 (m 3H); 2.19 (m 1H); 1.96-1.76 (m 2H); 1.66 (m 1H).

### Example 61

### {(S)-3-[5-(4-Bromo-1H-pyrrol-2-yl)-[1,2,4]oxadiazol-3-yl]-piperidin-1-yl}-(3-fluoro-pyridin-4-yl)-methanone

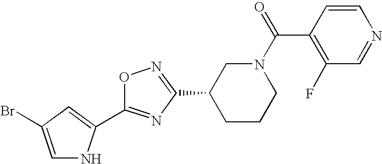

### 61(A) (S)-3-[5-(4-Bromo-1H-pyrrol-2-yl)-[1,2,4]oxadiazol-3-yl]-piperidine Hydrochloride

The title compound was prepared following the experimental procedure described in Example 28(A) and 28(B), starting from 4-bromo-1H-pyrrole-2-carboxylic acid, prepared as described in Example 44 (B).

Yield: 38%; LCMS (RT): 2.65 min (Method E); MS (ES+) gave m/z: 297.03 and 299.03.

### 61(B) {(S)-3-[5-(4-Bromo-1H-pyrrol-2-yl)-[1,2,4]oxadiazol-3-yl]-piperidin-1-yl}-(3-fluoro-pyridin-4-yl)-methanone

The title compound was prepared following the experimental procedure described in Example 28(C), starting from (S)-3-[5-(4-bromo-1H-pyrrol-2-yl)-[1,2,4]oxadiazol-3-yl]-piperidine hydrochloride, prepared as described in Example 61 (A), and using 3-fluoro-pyridine-4-carboxylic acid as the acid of choice.

Purification was performed by flash chromatography (silica gel, eluent: petroleum ether/ethyl acetate 1:2).

Yield: 79%; LCMS (RT): 3.12 min (Method P); MS (ES+) gave m/z: 419.9 (MH+).

¹H-NMR (DMSO-d₆ 373K), δ (ppm): 12.34 (s br, 1H); 8.61 (s, 1H); 8.50 (m, 1H); 7.44 (dd, 1H); 7.22 (d, 1H); 6.99 (s, 1H); 4.98-3.86 (m br, 2H); 3.41 (m, 1H); 3.27 (m, 1H); 3.10 (m, 1H); 2.21 (m, 1H); 2.01-1.80 (m, 2H); 1.65 (m, 1H).

### Example 62

### (3-Fluoro-pyridin-4-yl)-{(S)-3-[5-(4-fluoro-1H-pyrrol-2-yl)-[1,2,4]oxadiazol-3-yl]-piperidin-1-yl}-methanone

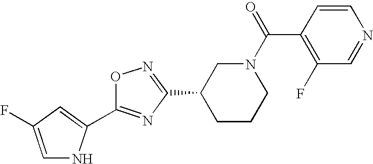

The title compound was prepared following the experimental procedure described in Example 28(C), starting from (S)-3-[5-(4-fluoro-1H-pyrrol-2-yl)-[1,2,4]oxadiazol-3-yl]-piperidine hydrochloride, prepared as described in Example 60 (13), and using 3-fluoro-pyridine-4-carboxylic acid as the acid of choice.

Purification was performed by flash chromatography (silica gel, eluent: DCM/MeOH 99:1).

Yield: 64%; LCMS (RT): 1.83 min (Method S); MS (ES+) gave m/z: 360.1 (MH+).

¹H-NMR (DMSO-d₆ 373K), δ (ppm): 11.87 (s br, 1H); 8.62 (s, 1H); 8.51 (m, 1H); 7.43 (dd, 1H); 7.01 (m, 1H); 6.76 (s br, 1H); 4.75-4.20 (m br, 2H); 3.41 (m, 1H); 3.28 (m, 1H); 3.10 (m, 1H); 2.20 (m, 1H); 2.01-1.79 (m, 2H); 1.64 (m, 1H).

### Example 63

### (4-Fluoro-phenyl)-{(S)-3-[5-(4-fluoro-1H-pyrrol-2-yl)-[1,2,4]oxadiazol-3-yl]-piperidin-1-yl}-methanone

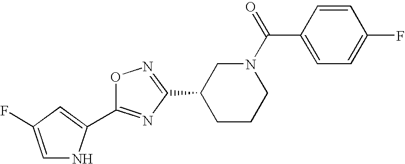

The title compound was prepared following the experimental procedure described in Example 1(C), starting from (S)-3-[5-(4-fluoro-1H-pyrrol-2-yl)-[1,2,4]oxadiazol-3-yl]-piperidine hydrochloride, prepared as described in Example 60 (B), and using 4-fluorobenzoyl chloride as the acylating agent.

Purification was performed by flash chromatography (silica gel, eluent:DCM/MeOH 98:2).

Yield: 31%; LCMS (RT): 2.21 min (Method S); MS (ES+) gave m/z: 359.1 (MH+).

¹H-NMR (DMSO-d₆ 353K), δ (ppm): 12.01 (s br 1H); 7.47 (dd 2H); 7.23 (dd 2H); 7.04 (m 1H); 6.68 (m 1H); 4.25 (m 1H); 3.83 (m 1H); 3.33 (dd 1H); 3.20 (ddd 1H); 3.09 (m 1H); 2.20 (m 1H); 1.96-1.77 (m 2); 1.64 (m 1H).

### Example 64

### (6-Fluoro-pyridin-3-yl)-{(S)-3-[5-(4-fluoro-1H-pyrrol-2-yl)-[1,2,4]oxadiazol-3-yl]-piperidin-1-yl}-methanone

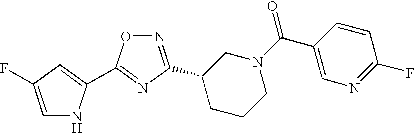

The title compound was prepared following the experimental procedure described in Example 28(C), starting from (S)-3-[5-(4-fluoro-1H-pyrrol-2-yl)-[1,2,4]oxadiazol-3-yl]-piperidine hydrochloride, prepared as described in Example 60 (13), and using 2-fluoro-pyridine-5-carboxylic acid as the acid of choice.

Purification was performed by flash chromatography (silica gel, eluent: petroleum ether/ethyl acetate 4:6) and then by a second column flash chromatography (silica gel, eluent: DCM).

Yield: 7% (gummy white solid); LCMS (RT): 1.99 min (Method S); MS (ES+) gave m/z: 360.1 (MH+).

¹H-NMR (DMSO-d₆ 353K), δ (ppm): 11.99 (s br 1H); 8.31 (m 1H); 8.02 (ddd 1H); 7.21 (ddd 1H); 7.05 (dd 1H); 6.78 (m 1H); 4.24 (m 1H); 3.80 (m 1H); 3.38 (dd 1H); 3.27 (ddd 1H); 3.13 (m 1H); 2.20 (m 1H); 1.97-1.77 (m 2H); 1.76 (m 1H).

### Example 65

### {(S)-3-[3-(4-Chloro-1H-pyrrol-2-yl)-[1,2,4]oxadiazol-5-yl]-piperidin-1-yl}-(6-fluoro-pyridin-3-yl)-methanone

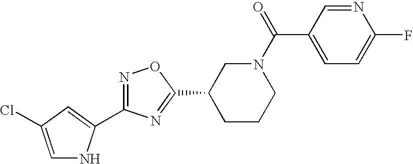

The title compound was prepared following the experimental procedure described in Example 28(C), starting from (S)-3-[3-(4-chloro-1H-pyrrol-2-yl)-[1,2,4]oxadiazol-5-yl]-piperidine hydrochloride, prepared as described in Example 43 (E), and using 2-fluoro-pyridine-5-carboxylic acid as the acid of choice.

Purification was performed by flash chromatography (silica gel, eluent: DCM/MeOH 40:1).

Yield: 56% (white amorphous solid); [α_{D}]=+125.0 (c=0.98; MeOH); LCMS (RT): 2.12 min (Method S); MS (ES+) gave m/z: 376.1 (MH+).

¹H-NMR (DMSO-d₆ 353K), δ (ppm): 1.88 (s br 1H); 8.13 (m 1H); 8.02 (ddd 1H); 7.22 (dd 1H); 7.04 (d 1H); 6.70 (d 1H); 4.23 (m 1H); 3.76 (m 1H); 3.55 (dd 1H); 3.41 (ddd 1H); 3.33 (ddd 1H); 2.25 (m 1H); 1.97 (m 1H); 1.82 (m 1H); 1.68 (m 1H).

### Example 66

### {(S)-3-[3-(4-Chloro-1H-pyrrol-2-yl)-[1,2,4]oxadiazol-5-yl]-piperidin-1-yl}-(2-fluoro-pyridin-4-yl)-methanone

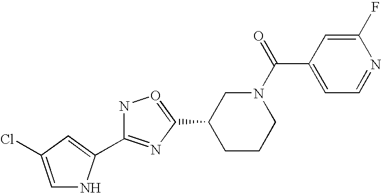

The title compound was prepared following the experimental procedure described in Example 28(C), starting from (S)-3-[3-(4-chloro-1H-pyrrol-2-yl)-[1,2,4]oxadiazol-5-yl]-piperidine hydrochloride, prepared as described in Example 43 (E), and using 2-fluoro-pyridine-4-carboxylic acid as the acid of choice.

Purification was performed by flash chromatography (silica gel, eluent: DCM/MeOH 40:1) and then by a successive column flash chromatography (silica gel, eluent: petroleum ether/ethyl acetate 2:1).

Yield: 66% (white amorphous solid); [α_{D}]=+120.6 (c 0.79; MeOH); LCMS (RT): 2.12 min (Method S); MS (ES+) gave m/z: 376.1 (MH+).

¹H-NMR (DMSO-d_{6,} 353K), δ (ppm): 11.90 (s br 1H); 8.33 (d 1H); 7.34 (m 1H); 7.16 (m 1H); 7.04 (d 1H); 6.70 (d 1H); 4.16 (m br 1H); 3.70 (m br 1H); 3.54 (dd 1H); 3.41 (m 1H); 3.30 (m 1H); 2.25 (m 1H); 1.96 (m 1H); 1.82 (m 1H); 1.67 (m 1H).

### Example 67

### {(S)-3-[3-(4-Chloro-1H-pyrrol-2-yl)-[1,2,4]oxadiazol-5-yl]-piperidin-1-yl}-(3-fluoro-pyridin-4-yl)-methanone

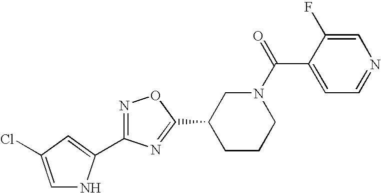

The title compound was prepared following the experimental procedure described in Example 28(C), starting from (S)-3-[3-(4-chloro-1H-pyrrol-2-yl)-[1,2,4]oxadiazol-5-yl]-piperidine hydrochloride, prepared as described in Example 43 (E), and using 3-fluoro-pyridine-4-carboxylic acid as the acid of choice.

Purification was performed by flash chromatography (silica gel, eluent: petroleum ether/ethyl acetate 2:1).

Yield: 84% (white amorphous solid); [α_{D}]=+107.7 (c=1.09; MeOH); LCMS (RT): 2.00 min (Method S); MS (ES+) gave m/z: 376.1 (MH+).

¹H-NMR (DMSO-d_{6,} 353K), δ (ppm): 11.90 (s br 1H); 8.65 (s 1H); 8.52 (dd 1H); 7.44 (dd 1H); 7.04 (d 1H); 6.70 (m br 1H); 4.51 (m br 1H); 4.07 (m br 1H); 3.57 (dd 1H); 3.38 (m 2H); 2.25 (m 1H); 1.99 (m 1H); 1.83 (m 1H); 1.66 (m 1H).

### Example 68

### {(S)-3-[3-(4-Chloro-1H-pyrrol-2-yl)-[1,2,4]oxadiazol-5-yl]-piperidin-1-yl}-(5-methyl-isoxazol-4-yl)-methanone

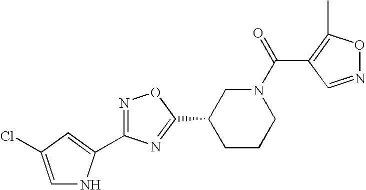

The title compound was prepared following the experimental procedure described in Example 28(C), starting from (S)-3-[3-(4-chloro-1H-pyrrol-2-yl)-[1,2,4]oxadiazol-5-yl]-piperidine hydrochloride, prepared as described in Example 43 (E), and using 5-methyl-isoxazole-4-carboxylic acid as the acid of choice.

Purification was performed by flash chromatography (silica gel, eluent: DCM/MeOH 40:1).

Yield: 38% (white amorphous solid); [α_{D}]=+95.1 (c=0.54; MeOH); LCMS IT): 2.09 min (Method S); MS (ES+) gave m/z: 362.1 (MH+).

¹H-NMR (DMSO-d_{6,} 373K), δ (ppm): 11.77 (s br 1H); 8.54 (s 1H); 7.02 (m 1H); 6.70 (m 1H); 4.23 (dd 1H); 3.79 (dd 1H); 3.57 (dd 1H); 3.37 (m 2H); 2.47 (d 3H); 2.25 (m 1H); 1.97 (m 1H); 1.85 (m 1H); 1.66 (m 1H).

### Example 69

### {(S)-3-[3-(4-Bromo-1h-pyrrol-2-yl)-[1,2,4]oxadiazol-5-yl]-piperidin-1-yl}-(3-fluoro-pyridin-4-yl)-methanone

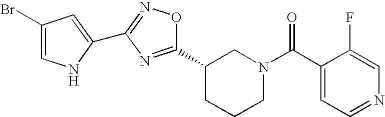

The title compound was prepared following the experimental procedure described in Example 28(C), starting from (S)-3-[3-(4-bromo-1H-pyrrol-2-yl)-[1,2,4]oxadiazol-5-yl]-piperidine hydrochloride, prepared as described in Example 45 (A), and using 3-fluoro-pyridine-4-carboxylic acid as the acid of choice.

Purification was performed by flash chromatography (silica gel, eluent gradient: from hexane/ethyl acetate 100:0 to hexane/ethyl acetate 0:100).

Yield: 60% (white amorphous solid); [α_{D}]=+100.3 (c=0.525, MeOH); LCMS (RT): 5.20 min (Method T); MS (ES+) gave m/z: 419.9 (MH+).

¹H-NMR (DMSO-d₆ 353K), δ (ppm): 11.97 (s br 1H); 8.64 (s 1H); 8.52 (dd 1H); 7.45 (dd 1H); 7.08 (m 1H); 6.76 (m br 1H); 4.51 (s br 1H); 4.06 (m br 1H); 3.57 (dd 1H); 3.37 (m 2H); 2.25 (m 1H); 1.99 (m 1H); 1.81 (m 1H); 1.64 (m 1H).

### Example 70

### (3-Fluoro-pyridin-4-yl)-{(S)-3-[3-(4-fluoro-1H-pyrrol-2-yl)-[1,2,4]oxadiazol-5-yl]-piperidin-1-yl}-methanone

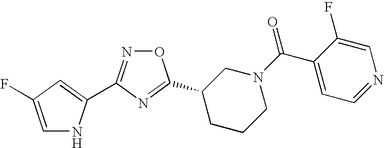

The title compound was prepared following the experimental procedure described in Example 28(C), starting from (S)-3-[3-(4-fluoro-1H-pyrrol-2-yl)-[1,2,4]oxadiazol-5-yl]-piperidine hydrochloride salt, prepared as described in Example 52 (A), and using 3-fluoro-pyridine-4-carboxylic acid as the acid of choice.

Yield: 40% (white solid); LCMS (RT): 1.83 min (Method S); MS (ES+) gave m/z: 360.1 (MH+).

¹H-NMR (DMSO-d₆ 353K), δ (ppm): 11.46 (s br 1H); 8.64 (s 1H); 8.52 (dd 1H); 7.45 (dd 1H); 6.86 (m 1H); 6.54 (m br 1H); 4.49 (m br 1H); 4.07 (m br 1H); 3.56 (dd 1H); 3.34 (m 2H); 2.25 (m 1H); 1.99 (m 1H); 1.82 (m 1H); 1.64 (m 1H).

### Example 71

### (3-Fluoro-pyridin-4-yl)-{(S)-3-[3-(4-methyl-1H-pyrrol-2-yl)-[1,2,4]oxadiazol-5-Yl]-piperidin-1-yl}-methanone

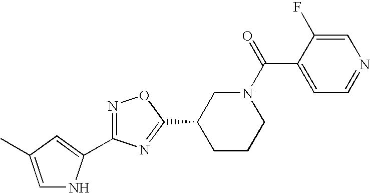

The title compound was prepared following the experimental procedure described in Example 28(C), starting from (S)-3-[3-(4-methyl-1H-pyrrol-2-yl)-[1,2,4]oxadiazol-5-yl]-piperidine trifluoroacetate, prepared as described in Example 31 (E), and using 3-fluoro-pyridine-4-carboxylic acid as the acid of choice.

Purification was performed by flash chromatography (silica gel, eluent: petroleum ether/ethyl acetate 2:1).

Yield: 65% (white amorphous solid); [α_{D}]=+112.1 (c=0.80; MeOH); LCMS (RT): 1.89 min (Method S); MS (ES+) gave m/z: 356.1 (MH+).

¹H-NMR (DMSO-d_{6,} 353K), δ (ppm): 11.16 (s br 1H); 8.65 (s 1H); 8.52 (dd 1H); 7.45 (dd 1H); 6.74 (s 1H); 6.57 (m br 1H); 4.51 (m br 1H); 4.06 (m br 1H); 3.56 (dd 1H); 3.34 (m br 2H), 2.24 (m 1H); 2.08 (s 3H); 1.98 (m 1H); 1.82 (m 1H); 1.64 (m 1H).

### Example 72

### (4-Fluoro-phenyl)-{(S)-3-[5-(4-cyano-1H-pyrrol-2-yl)-[1,2,4]oxadiazol-3-yl]-piperidin-1-yl}-methanone

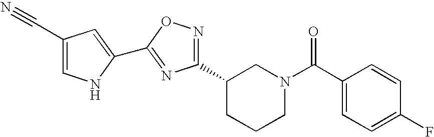

### 72(A) 5-(2,2,2-Trichloro-acetyl)-1H-pyrrole-3-carbonitrile

A solution of 2,2,2-trichloro-1-(1H-pyrrol-2-yl)-ethanone (1.5 g, 7 mmol) (prepared as described in Belanger; Tetrahedron Lett.; 1979; 2505-2508) in MeCN (15 mL) was cooled to 0° C. and chlorosulfonyl isocyanate (1.32 mL, 15 mmol) was added. The solution was allowed to warm to room temperature and stirred for 3 hours under N₂, then DMF (5 mL) was added and the solution stirred overnight. Water was added and the solution extracted three times with DCM. The combined organic extracts were washed with 5% NaHCO₃ solution and the solvent removed. The residue was purified by flash chromatography (silica gel cartridge, eluent gradient: from hexane/ethyl acetate 100:0 to hexane/ethyl acetate 40:60) to give the product as a pale yellow solid.

Yield: 85%; LCMS (RT): 5.0 min (Method D); MS (ES+) gave m/z: 237 (MH+).

¹H-NMR (CDCl₃); δ (ppm): 9.72 (s br, 1H); 7.10 (s, 1H); 7.09 (s, 1H).

### 72(B) (4-Fluoro-phenyl)-{(S)-3-[5-(4-cyano-1H-pyrrol-2-yl)-[1,2,4]oxadiazol-3-yl]-piperidin-1-yl}-methanone

5-(2,2,2-Trichloro-acetyl)-1H-pyrrole-3-carbonitrile (150 mg, 0.63 mmol), (S)-1-(4-fluoro-benzoyl)-N-hydroxy-piperidine-3-carboxamidine (167 mg, 0.63 mmol) (prepared as described in Example 27(D)), and triethylamine (100 μL, 0.72 mmol) were dissolved in MeCN and heated in a sealed tube in a microwave reactor for 15 min at 100° C., then 1 hour at 100° C., then 30 min at 120° C. The solvent was removed and the residue was purified by flash chromatography (silica gel cartridge, eluent gradient: from hexane/ethyl acetate 100:0 to hexane/ethyl acetate 20:80) to give the product as a colourless gum which was then recrystallised from DCM/hexane to give the product as a white solid.

Yield: 26%; mp=204.8-205.6° C.; [α_{D}]=+87 (c=0.42, MeOH); LCMS (RT): 2.62 min (method S); MS (ES+) gave m/z: 366.3 (MH+).

¹H-NMR (DMSO-d₆ 353K), δ (ppm): 13.06 (s br, 1H); 7.87 (d, 1H); 7.46 (dd, 2H); 7.37 (d, 1H); 7.23 (dd, 2H); 4.27 (m, 1H); 3.83 (m, 1H); 3.34 (dd, 1H); 3.21 (ddd, 1H); 3.13 (ddd, 1H); 2.21 (m, 1H); 1.97-1.77 (m, 2H); 1.62 (m, 1H).

### Example 73

### 5-{3-[(S)-1-(6-Fluoro-pyridine-3-carbonyl)-piperidin-3-yl]-[1,2,4]oxadiazol-5-yl}-1H-pyrrole-3-carbonitrile

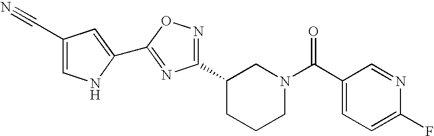

### 73 (A) (S)-3-[5-(4-Cyano-1H-pyrrol-2-yl)-[1,2,4]oxadiazol-3-yl]-piperidine-1-carboxylic Acid tert-butyl Ester

A solution of 5-(2,2,2-trichloro-acetyl)-1H-pyrrole-3-carbonitrile (750 mg, 4.19 mmol) (prepared as described in Belanger; Tetrahedron Lett.; 1979; 2505-2508), (S)-3-(N-hydroxycarbamimidoyl)-piperidine-1-carboxylic acid tert-butyl ester (730 mg, 4.11 mmol) (prepared as described in Example 10(C)), and triethylamine (500 μL, 7.2 mmol) in MeCN (40 mL) was refluxed for 3 hours then the solvent removed. The residue was purified by flash chromatography (silica gel cartridge, eluent gradient: from hexane/ethyl acetate 100:0 to hexane/ethyl acetate 40:60) to give a white solid. This intermediate was dissolved in MeCN (2 mL) and heated in a sealed tube in a microwave reactor at 100° C. for 30 min then at 120° C. for 1 hour. The solution was passed through an SCX cartridge (eluting with MeOH), then the solvent was removed. The residue was purified by flash chromatography (silica gel cartridge, eluent gradient: from hexane/ethyl acetate 100:0 to hexane/ethyl acetate 50:50) to give the product as a white solid.

Yield: 21%; LCMS (RT): 2.46 min (Method I); MS (ES+) gave m/z: 344 (MH+).

### 73(B) (S)-3-[5-(4-Cyano-1H-pyrrol-2-yl)-[1,2,4]oxadiazol-3-yl]-piperidine Hydrochloride Salt

(S)-3-[5-(4-Cyano-1H-pyrrol-2-yl)-[1,2,4]oxadiazol-3-yl]-piperidine-1-carboxylic acid tert-butyl ester (300 mg, 0.9 mmol) was dissolved in 4M HCl in dioxane (3 mL) and stirred at room temperature under N₂ for 90 minutes. The solvent was removed and the residue dried under high vacuum to give the product as a white solid.

Yield: 100%; LCMS (RT): 1.15 min (Method I); MS (ES+) gave m/z: 244 (MH+).

### 73(C) 5-{3-[(S)-1-(6-Fluoro-pyridine-3-carbonyl)-piperidin-3-yl]-[1,2,4]oxadiazol-5-yl}-1H-pyrrole-3-carbonitrile

A mixture of 6-fluoro nicotinic acid (50 mg, 0.35 mmol), HOAT (55 mg, 0.4 mmol), EDCI.HCl (77 mg, 0.4 mmol) in dry DCM (10 mL) was stirred at room temperature under N₂ for 10 minutes, then (S)-3-[5-(4-cyano-1H-pyrrol-2-yl) -[1,2,4]oxadiazol-3-yl]-piperidine hydrochloride salt (81 mg, 0.3 mmol) and triethylamine (110 μL, 0.8 mmol) were added and the solution stirred overnight at room temperature. The solution was washed with water and 0.2M NaOH solution, dried and the solvent removed to give a residue that was purified by flash chromatography (silica gel cartridge, eluent gradient: from hexane/ethyl acetate 100:0 to hexane/ethyl acetate 30:70) to give the product as a colourless gum.

Yield: 38%; LCMS (RT): 4.14 min (Method D); MS (ES+) gave m/z: 367.1 (MH+).

### Example 74

### 5-{3-[(S)-1-(2-Fluoro-pyridine-4-carbonyl)-piperidin-3-yl]-[1,2,4]oxadiazol-5-yl}-1H-pyrrole-3-carbonitrile

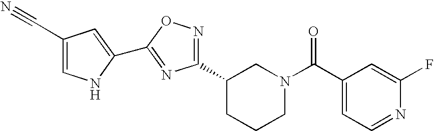

A mixture of 2-fluoro isonicotinic acid (50 mg, 0.35 mmol), HOAT (55 mg, 0.4 mmol), EDCI.HCl (77 mg, 0.4 mmol) in dry DCM (10 mL) was stirred at room temperature under N₂ for 10 minutes, then (S)-3-[5-(4-cyano-1H-pyrrol-2-yl)-[1,2,4]oxadiazol-3-yl]-piperidine hydrochloride salt (81 mg, 0.3 mmol) (prepared as described in Example 73(B)) and triethylamine (110 μL, 0.8 mmol) were added and the solution stirred overnight at room temperature. The solution was washed with water and 0.2 M NaOH solution, dried and the solvent removed to give a residue that was purified by flash chromatography (silica gel cartridge, eluent gradient: from hexane/ethyl acetate 100:0 to hexane/ethyl acetate 30:70) to give the product as a colourless gum.

Yield: 91%; LCMS (RT): 4.16 min (Method D); MS (ES+) gave m/z: 367.1 (MH+).

### Example 75

### 5-{3-[(S)-1-(3-Fluoro-pyridine-4-carbonyl)-piperidin-3-yl]-[1,2,4]oxadiazol-5-yl}-1H-pyrrole-3-carbonitrile

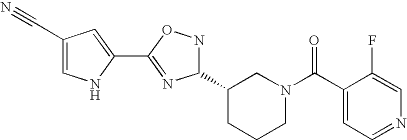

A mixture of 3-fluoro isonicotinic acid (50 mg, 0.35 mmol), HOAT (55 mg, 0.4 mmol), EDCI.HCl (77 mg, 0.4 mmol) in dry DCM (10 mL) was stirred at room temperature under N₂ for 10 minutes, then (S)-3-[5-(4-cyano-1H-pyrrol-2-yl)-[1,2,4]oxadiazol-3-yl]-piperidine hydrochloride salt (81 mg, 0.3 mmol) (prepared as described in Example 73(B)) and triethylamine (110 μL, 0.8 mmol) were added and the solution stirred overnight. The solution was washed with water and 0.2 M NaOH solution, dried and the solvent removed to give a residue that was purified by flash chromatography (silica gel cartridge, eluent gradient: from hexane/ethyl acetate 100:0 to hexane/ethyl acetate 30:70) to give the product as a colourless gum.

Yield: 61%; LCMS (RT): 3.91 min (Method D); MS (ES+) gave m/z: 367.1 (MH+).

### Example 76

### (4-Fluoro-phenyl)-{(S)-3-[5-(4-trifluoromethyl-1H-pyrrol-2-yl)-[1,2,4]oxadiazol-3-yl]-piperidin-1-yl}-methanone

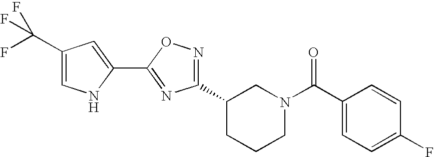

### 76(A) 4-Trifluoromethyl-pyrrole-1,2-dicarboxylic Acid 2-benzyl Ester 1-tert-butyl Ester

The title compound was prepared according to the procedures reported in X. Qui, F. Qing, J. Org. Chem. 2002, 67, 7162-7164; and X. Qui, F. Qing, J. Org. Chem. 2003, 68, 3614-3617.

### 76(B) (S)-3-[5-(4-Trifluoromethyl-1H-pyrrol-2-yl)-[1,2,4]oxadiazol-3-yl]-piperidine-1-carboxylic Acid tert-butyl Ester

4-Trifluoromethyl-pyrrole-1,2-dicarboxylic acid 2-benzyl ester 1-tert-butyl ester (498 mg, 1.35 mmol) was suspended in 4M HCl in dioxane (4 ml) and the mixture was stirred at room temperature for 6 hours. Then the solvent was removed affording a pale yellow solid, which was dissolved in EtOH (15 ml) and hydrogenolysed at 20 psi, at room temperature, in the presence of 10% Pd/C (40 mg) for 2 hours. Catalyst was filtered off and the filtrate was concentrated to dryness affording 220 mg of an off-white solid. A mixture of this product (163 mg, 0.91 mmol), HOAT (149 mg, 1.1 mmol), EDCI.HCl (211 mg, 1.1 mmol) in dry DCM (20 mL) was kept under stirring at ambient temperature for 30 minutes under nitrogen atmosphere. Then, (S)-3-(N-hydroxycarbamimidoyl)-piperidine-1-carboxylic acid tert-butyl ester (204 mg, 0.84 mmol) (prepared as described in Example 10(C)) was added and stirring at RT was maintained overnight. The reaction mixture was diluted with DCM and washed with water, then with 5% citric acid (aq) and NaHCO₃ satd. solution (aq). The organic layer was separated, dried over Na₂SO₄ and concentrated to dryness affording a beige solid (261 mg). This solid (250 mg) was suspended in CH₃CN (3 ml) and heated at 100° C. under microwaves irradiation for 3 hours, in a sealed tube. Then, the solution was concentrated in vacuo and the residue purified by flash chromatography (silica gel, eluent: petroleum ether/ethyl acetate 60:40) affording 192 mg of a white solid.

Yield: 55% (over 4 steps); LCMS (RT): 8.2 min (Method M), MS (ES+) gave m/z: 409.0 (M+23), 287.0 (M-99).

### 76(C) 3-[5-(4-Trifluoromethyl-1H-pyrrol-2-yl)-[1,2,4]oxadiazol-3-yl]-piperidine Hydrochloride

(S)-3-[5-(4-Trifluoromethyl-1H-pyrrol-2-yl)-[1,2,4]oxadiazol-3-yl]-piperidine-1-carboxylic acid tert-butyl ester (192 mg, 0.5 mmol) was dissolved in 4M HCl in dioxane (2 mL), and the reaction mixture was stirred at room temperature for 1 h. The solvent was evaporated under reduced pressure to give the title compound, which was used for the next step without further purification.

Yield: quantitative; LCMS (RT): 1.39 min (Method L); MS (ES+) gave m/z: 287.0 (M+1).

### 76(D) (4-Fluoro-phenyl)-{(S)-3-[5-(4-trifluoromethyl-1H-pyrrol-2-yl)-[1,2,4]oxadiazol-3-yl]-piperidin-1-yl}-methanone

A mixture of 3-[5-(4-trifluoromethyl-1H-pyrrol-2-yl)-[1,2,4]oxadiazol-3-yl]-piperidine hydrochloride (73 mg, 0.22 mmol), 4-fluorobenzoyl chloride (26 μl, 0.22 mmol) and triethylamine (68 μl, 0.48 mmol) in DCM (7 ml), was stirred at room temperature overnight. The reaction mixture was concentrated and the residue was purified by flash chromatography (silica gel, eluent: petroleum ether/ethyl acetate 60:40) affording 71 mg of a white solid.

Yield: 79% (white solid); [α]_{D}²⁰=+94.3 (c=1.0, MeOH); mp=183.5° C.; LCMS (RT): 2.49 min (Method S); MS (ES+) gave m/z: 408.9 (MH+).

¹H-NMR (DMSO-d₆ 353K), δ (ppm): 12.83 (s br, 1H); 7.62 (m, 1H); 7.47 (dd, 2H); 7.22 (dd, 2H); 7.21 (m, 1H); 4.28 (m, 1H); 3.83 (m, 1H); 3.35 (dd, 1H); 3.22 (ddd, 1H); 3.13 (ddd, 1H); 2.21 (m, 1H); 1.97-1.78 (m, 2H); 1.63 (m, 1H).

### Example 77

### (3-Fluoro-pyridin-4-yl)-{(S)-3-[5-(4-trifluoromethyl-1H-pyrrol-2-yl)[1,2,4]oxadiazol-3-yl]-piperidin-1-yl}-methanone

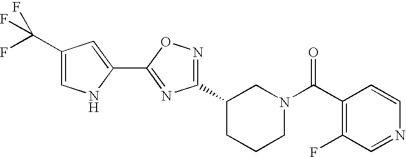

A mixture of 3-fluoro-isonicotinic acid (43 mg, 0.30 mmol) HOAT (50 mg, 0.37 mmol), EDCI.HCl (71 mg, 0.37 mmol) in dry DCM (8 mL) was kept under stirring at ambient temperature for 2 hours under nitrogen atmosphere. The reaction mixture was added to a solution of 3-[5-(4-trifluoromethyl-1H-pyrrol-2-yl)-[1,2,4]oxadiazol-3-yl]-piperidine hydrochloride (93 mg, 0.28 mmol), prepared as described in Example 76(C), and triethylamine (50 μL, 0.37 mmol) in DCM (2 mL) and the solution was kept under stirring at ambient temperature overnight. Then the reaction mixture was diluted with DCM and washed with water. The organic layer was separated, dried over Na₂SO₄ and concentrated. Flash chromatography purification of the crude (silica gel, eluent: petroleum ether/ethyl acetate 15:85) afforded 66 mg of a white foam.

Yield: 57% (white foam); [α]_{D}²⁰=+76.4 (c=0.5, MeOH); LCMS (RT): 2.15 min (Method S); MS (ES+) gave m/z: 410.1 (MH+).

¹H-NMR (DMSO-d_{6,} 373 K), δ (ppm): 12.70 (s br, 1H); 8.61 (s, 1H); 8.50 (dd, 1H); 7.59 (m, 1H); 7.43 (dd, 1H); 7.19 (s br, 1H); 4.86-3.65 (m br, 2H); 3.42 (m, 1H); 3.28 (m, 1H); 3.13 (m, 1H); 2.22 (m, 1H); 2.01-1.80 (m, 2H); 1.65 (m, 1H).

### Example 78

### (6-Fluoro-pyridin-3-yl)-{(S)-3-[5-(4-trifluoromethyl-1H-pyrrol-2-yl)-[1,2,4]oxadiazol-3-yl]-piperidin-1-yl}-methanone

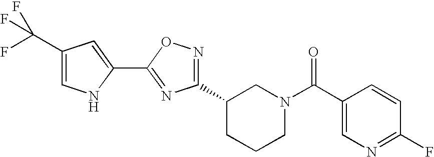

The compound was prepared following the procedure described in the Example 77, starting from 3-[5-(4-trifluoromethyl-1H-pyrrol-2-yl)-[1,2,4]oxadiazol-3-yl]-piperidine hydrochloride (93 mg, 0.28 mmol), prepared as described in Example 76(C), and using 6-fluoro-nicotinic acid (43 mg, 0.30 mmol) as the acid of choice. The final compound was purified by flash chromatography (silica gel, eluent: petroleum ether/ethyl acetate 30:70).

Yield: 38% (off-white solid); [α]_{D}²⁰=+124.0 (c=0.5, MeOH); mp=165.7° C.; LCMS (RT): 2.26 min (Method S); MS (ES+) gave m/z: 410.1 (MH+).

¹H-NMR (DMSO-d_{6,} 353K), δ (ppm): 12.80 (s br, 1H); 8.31 (ddd, 1H); 8.03 (ddd, 1H); 7.62 (m, 1H); 7.22 (m, 1H); 7.21 (ddd, 1H); 4.26 (m, 1H); 3.81 (m, 1H); 3.41 (dd, 1H); 3.28 (ddd, 1H); 3.17 (ddd, 1H); 2.22 (m, 1H); 2.00-1.78 (m, 2H); 1.68 (m, 1H).

### Example 79

### (3,4-Difluoro-phenyl)-{(S)-3-[3-(4-methyl-1H-imidazol-2-yl)-[1,2,4]oxadiazol-5-yl]-piperidin-1-yl}-methanone

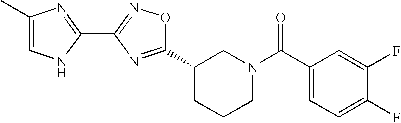

### 79(A) N-Hydroxy-4-methyl-1H-imidazole-2-carboxamidine

A solution of 4-methyl-1H-imidazole-2-carbonitrile (83 mg, 0.776 mmol), prepared according to Helvetica Chimica Acta, 2005, 88, 2454-2469, and NH₂OH (50% water, 0.191 ml, 3.104 mmol) in absolute ethanol (2 ml) was heated at reflux for 1.5 h. The solvent was evaporated to give 110 mg of amorphous solid that was used in the next step without further purification.

Yield: quantitative; LC-MS (T): 0.31 min (Method H), MS (ES+) gave m/z: 140.9 (MH+).

### 79(B) (S)-3-[3-(4-Methyl-1H-imidazol-2-yl)-[1,2,4]oxadiazol-5-yl]-piperidine-1-carboxylic Acid tert-butyl Ester

HOBT (118 mg, 0.776 mmol) and EDC (222 mg, 1.164 mmol) were added to a stirred solution of (S)-N-Boc-nipecotic acid (177 mg, 0.776 mmol) in dioxane (1.5 ml) at room temperature. After 1 h, a solution of N-hydroxy-4-methyl-1H-imidazole-2-carboxamidine (0.776 mmol) in dioxane (3 ml) was added and the mixture stirred at RT for 24 b. Ethyl acetate was added and the mixture was washed with 5% NaHCO₃ (aq); the organic phase was dried over Na₂SO₄ and concentrated. The crude was purified by flash chromatography (silica gel cartridge, eluent: ethyl acetate/petroleum ether 2:1) to give 240 mg of pure product.

A mixture of the obtained product (240 mg, 0.683 mmol) and molecular sieves (4 A, 50 mg) in acetonitrile (3 ml) was heated at 130° C. for 3 h in a sealed tube, under microwave irradiation. Molecular sieves were filtered off and the solution was concentrated. The crude was purified by flash chromatography (silica gel cartridge, eluent: ethyl acetate/petroleum ether 2:1) to give 152 mg of title compound (transparent viscous oil).

Yield: 67%; LC-MS (RT): 1.05 min (Method H), MS (ES+) gave m/z: 334.0 (MH+).

### 79(C) (S)-3-[3-(4-Methyl-1H-imidazol-2-yl)-[1,2,4]oxadiazol-5-yl]-piperidine Dihydrochloride

A mixture of S)-3-[3-(4-methyl-1H-imidazol-2-yl)-[1,2,4]oxadiazol-5-yl]-piperidine-1-carboxylic acid tert-butyl ester (152 mg, 0.456 mmol) and HCl (4M dioxane solution, 0.57 ml) in dichloromethane (3 ml) was stirred at room temperature for 20 h. The solvent was evaporated to give a white solid (140 mg) that was used in the next step without further purification.

Yield: quantitative; LC-MS (RT): 0.32 min (Method H), MS (ES+) gave m/z: 234.1 (MH+).

### 79(D) (3,4-Difluoro-phenyl)-{(S)-3-[3-(4-methyl-1H-imidazol-2-yl)-[1,2,4]oxadiazol-5-yl]-piperidin-1-yl}-methanone

A mixture of 3,4-difluoro-benzoyl chloride (0.057 ml, 0.456 mmol) in 2 ml of dichloromethane was added to a stirred solution of (S)-3-[3-(4-methyl-1H-imidazol-2-yl)-[1,2,4]oxadiazol-5-yl]-piperidine dihydrochloride (140 mg, 0.456 mmol) and triethylamine (0.255 ml, 1.824 mmol) in 2 ml of dichloromethane at 0° C. After 30 min the solvent was evaporated, the residue was partitioned between ethyl acetate and 5% NaHCO₃ (aq). The aqueous phase was separated and extracted twice with ethyl acetate; the combined organic layers were dried over Na₂SO₄ and concentrated. The crude was purified by flash chromatography (silica gel cartridge, eluent: dichloromethane/methanol 20/0.8) to give 118 mg of title compound (amorphous solid).

Yield: 69%. LCMS (RT): 1.92 min (Method N); MS (ES+) gave m/z: 374.3 (MH+).

¹H-NMR (DMSO-d_{6,} 353 K), δ (ppm): 12.58 (s br, 1H); 7.53-7.40 (m, 2H); 7.28 (m, 1H); 6.93 (s, 1H); 4.22 (m, 1H); 3.76 (m, 1H); 3.53 (dd, 1H); 3.42 (ddd, 1H); 3.29 (ddd, 1H); 2.27 (m, 1); 2.24 (s, 3H); 1.98 (m, 1H); 1.83 (m, 1H); 1.66 (m, 1H).

### Example 80

### {(S)-3-[5-(4-Chloro-1H-pyrrol-2-yl)-[1,2,4]oxadiazol-3-yl]-piperidin-1-yl}-pyridin-4-yl-methanone

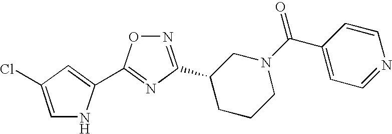

The title compound was prepared following the experimental procedure described in Example 28(C), starting from (S)-3-[5-(4-chloro-1H-pyrrol-2-yl)-[1,2,4]oxadiazol-3-yl]-piperidine hydrochloride, prepared as described in Example 39 (C), and using isonicotinic acid as the acid of choice.

Purification was performed by flash chromatography (silica gel, eluent: petroleum ether/ethyl acetate 2:8+1% NH₄OH).

Yield: 38% (gummy white solid); LCMS (RT): 1.62 min (Method S); MS (ES+) gave m/z: 358.1 (MH+).

### Example 81

### (6-Fluoro-pyridin-3-yl)-{(S)-3-[3-(4-trifluoromethyl-1H-pyrrol-2-yl)-[1,2,4]oxadiazol-5-yl]-piperidin-1-yl}-methanone

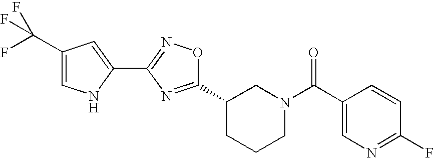

### 81(A) 4-Trifluoromethyl-1H-pyrrole-2-carboxylic Acid Amide

Carbonyl diimidazole (379 mg, 2.34 mmol) was added to a solution of 4-trifluoromethyl-1H-pyrrole-2-carboxylic acid (350 mg, 1.95 mmol) in MeCN (10 mL) and stirred for 90 min. Concentrated NH₄OH solution (2 mL) was added and the resulting mixture refluxed for 90 min. The solvent was removed, 10% citric acid solution (10 mL) was added and the solution extracted three times with EtOAc. The organic extracts were combined, dried over sodium sulphate and the solvent removed to give the product as a syrup.

Yield: 100% LCMS (RT): 1.29 min (Method L); MS (ES+) gave m/z: 178.9 (MH+).

### 81(B) 4-Trifluoromethyl-N-hydroxy-1H-pyrrole-2-carboxamidine

A solution of 4-Trifluoromethyl-1H-pyrrole-2-carboxylic acid amide (347 mg, 1.95 mmol) in phosphorus oxychloride (5 mL) was heated at 100° C. for 5 minutes, cooled, ice was added, basified with conc. NH₄OH solution then extracted three times with EtOAc. The organic extracts were combined, dried and the solvent removed to give a pale brown oil. This product was treated with 50% Hydroxylamine solution in water (1.2 mL, 20 mmol) and heated under reflux for 1 h. The solvent was removed under vacuum and the residue purified by flash chromatography (silica gel cartridge, eluent gradient: from hexane/ethyl acetate 100:0 to hexane/ethyl acetate 0:100) to give the product as a syrup.

Yield: 42% LCMS (RT): 0.93 min (Method L); MS (ES+) gave m/z: 193.9 (MH+).

### 81 (C) (S)-3-[3-(4-Trifluoromethyl-1H-pyrrol-2-yl)-[1,2,4]oxadiazol-5-yl]-piperidine-1-carboxylic Acid tert-butyl Ester

A mixture of (S)-N-Boc-nipecotic acid (206 mg, 0.90 mmol), HOAT (147 mg, 1.08 mmol), EDCI.HCl (207 mg, 1.08 mmol) in dry DCM (15 mL) was stirred under N₂ for 45 minutes, then 4-Trifluoromethyl-N-hydroxy-1H-pyrrole-2-carboxamidine (160 mg, 0.83 mmol) was added and the solution stirred 3 hours. The solution was washed with water, 10% citric acid solution and 5% NaHCO₃ solution, dried over sodium sulphate and the solvent removed to give a residue that was purified by flash chromatography (silica gel cartridge, eluent gradient: from hexane/ethyl acetate 100:0 to hexane/ethyl acetate 80:20). The solid thus obtained was dissolved in acetonitrile (2 mL) and heated in a sealed tube at 80° C. for 75 min in a microwave reactor. The solvent was removed and the crude residue was purified by flash chromatography (silica gel, petroleum ether/ethyl acetate 70:30) to give the product as a syrup.

Yield: 43%; LCMS (RT): 2.66 min (Method L); MS (ES+) gave m/z: 408.9 (MNa+).

### 81(D) (S)-3-[3-(4-Trifluoromethyl-1H-pyrrol-2-yl)-[1,2,4]oxadiazol-5-yl]-piperidine Hydrochloride Salt

(S)-3-[3-(4-Trifluoromethyl-1H-pyrrol-2-yl)-[1,2,4]oxadiazol-5-yl]-piperidine-1-carboxylic acid tert-butyl ester (140 mg, 0.36 mmol) was dissolved in 4M HCl in dioxane (2 mL), and the reaction mixture was stirred at room temperature for 1 h. The solvent was evaporated under reduced pressure to give the title compound, which was used for the next step without further purification.

Yield: quantitative; LCMS (RT): 1.38 min (Method L); MS (ES+) gave m/z: 286.9 (M+1).

### 81 (E) (4-Fluoro-phenyl)-{(S)-3-[3-(4-Trifluoromethyl-1H-pyrrol-2-yl)-[1,2,4]oxadiazol-5-yl]-piperidin-1-yl}-methanone

A mixture of 6-Fluoro-nicotinic acid (37 mg, 0.26 mmol), HOAT (38 mg, 0.28 mmol), EDCI.HCl (55 mg, 0.28 mmol) in dry DCM (8 mL) was kept under stirring at ambient temperature for 1.5 hours under nitrogen atmosphere. The reaction mixture was added to a solution of (S)-3-[3-(4-Trifluoromethyl-1H-pyrrol-2-yl)-[1,2,4]oxadiazol-5-yl]-piperidine hydrochloride salt (77 mg, 0.24 mmol) and triethylamine (73 uL, 0.54 mmol) in DCM (2 mL) and the solution was kept under stirring at ambient temperature overnight. Then the reaction mixture was diluted with DCM and washed with water. The organic layer was separated, dried over Na₂SO₄ and concentrated. Flash chromatography purification of the crude (silica gel, petroleum ether/ethyl acetate 50:50) afforded 72 mg of a gummy solid.

Yield: 73%; LCMS (RT): 2.12 min (Method L); MS (ES+) gave m/z: 409.8 (MH+), 431.9 (M-Na+).

### Pharmacology:

The compounds provided in the present invention are positive allosteric modulators of mGluR5. As such, these compounds do not appear to bind to the orthosteric glutamate recognition site, and do not activate the mGluR5 by themselves. Instead, the response of mGluR5 to a concentration of glutamate or mGluR5 agonist is increased when compounds of Formula I are present. Compounds of Formula I are expected to have their effect at mGluR5 by virtue of their ability to enhance the function of the receptor.

### Example A

### mGluR5 Assay on Rat Cultured Cortical Astrocytes

Under exposure to growth factors (basic fibroblast growth factor, epidermal growth factor), rat cultured astrocytes express group I-Gq coupled mGluR transcripts, namely mGluR5, but none of the splice variants of mGluR1, and as a consequence, a functional expression of mGluR5 receptors (Miller et al. (1995) J. Neurosci. 15:6103-9): The stimulation of mGluR5 receptors with selective agonist CHPG and the full blockade of the glutamate-induced phosphoinositide (PI) hydrolysis and subsequent intracellular calcium mobilization with specific antagonist as MPEP confirm the unique expression of mGluR5 receptors in this preparation.

This preparation was established and used in order to assess the properties of the compounds of the present invention to increase the Ca²⁺ mobilization-induced by glutamate without showing any significant activity when applied in the absence of glutamate.

### Primary Cortical Astrocytes Culture:

Primary glial cultures were prepared from cortices of Sprague-Dawley 16 to 19 days old embryos using a modification of methods described by Mc Carthy and de Vellis (1980) J. Cell Biol. 85:890-902 and Miller et al. (1995) J. Neurosci. 15 (9):6103-9. The cortices were dissected and then dissociated by trituration in a sterile buffer containing 5.36 mM KCl, 0.44 mM NaHCO₃, 4.17 mM KH₂PO₄, 137 mM NaCl, 0.34 mM NaH₂PO₄, 1 g/L glucose. The resulting cell homogenate was plated onto poly-D-lysine precoated T175 flasks (BIOCOAT, Becton Dickinson Biosciences, Erembodegem, Belgium) in Dubelcco's Modified Eagle's Medium (D-MEM GlutaMAX™ I, Invitrogen, Basel, Switzerland) buffered with 25 mM HEPES and 22.7 mM NaHCO₃, and supplemented with 4.5 g/L glucose, 1 mM pyruvate and 15% fetal bovine serum (FBS, Invitrogen, Basel, Switzerland), penicillin and streptomycin and incubated at 37° C. with 5% CO₂. For subsequent seeding, the FBS supplementation was reduced to 10%. After 12 days, cells were subplated by trypsinisation onto poly-D-lysine precoated 384-well plates at a density of 20.000 cells per well in culture buffer.

### Ca²⁺ Mobilization Assay Using Rat Cortical Astrocytes:

After one day of incubation, cells were washed with assay buffer containing: 142 mM NaCl, 6 mM KCl, 1 mM Mg₂SO₄, 1 mM CaCl₂, 20 mM HEPES, 1 g/L glucose, 0.125 mM sulfinpyrazone, pH 7.4. After 60 min of loading with 4 μM Fluo-4 (TefLabs, Austin, Tex.), the cells were washed three times with 50 μl of PBS Buffer and resuspended in 45 μl of assay Buffer. The plates were then transferred to a Fluorometric Imaging Plate Reader (FLIPR, Molecular Devices, Sunnyvale, Calif.) for the assessment of intracellular calcium flux. After monitoring the baseline fluorescence for 10 s, a solution containing 10 μM of representative compound of the present invention diluted in Assay Buffer (15 μl of 4× dilutions) was added to the cell plate in the absence or in the presence of 300 nM of glutamate. Under these experimental conditions, this concentration induces less than 20% of the maximal response of glutamate and was the concentration used to detect the positive allosteric modulator properties of the compounds from the present invention. The final DMSO concentration in the assay was 0.3%. In each experiment, fluorescence was then monitored as a function of time for 3 minutes and the data analyzed using Microsoft Excel and GraphPad Prism. Each data point was also measured two times.

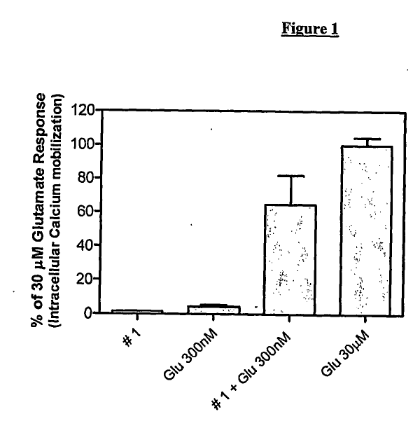

The results in FIG. 1 represent the effect of 10 μM of Example #1 on primary cortical mGluR5-expressing cell cultures in the absence or in the presence of 300 nM glutamate. Data are expressed as the percentage of maximal response observed with 30 μM glutamate applied to the cells. Each bar graph is the mean and S.E.M of duplicate data points and is representative of three independent experiments

The results shown in Example A demonstrate that the compounds described in the present invention do not have an effect per se on mGluR5. Instead, when compounds are added together with an mGluR5 agonist such as glutamate, the effect measured is significantly potentiated compared to the effect of the agonist alone at the same concentration. This data indicates that the compounds of the present invention are positive allosteric modulators of mGluR5 receptors in native preparations.

### Example B

### mGluR5 Assay on HEK-Expressing Rat mGluR5

### Cell Culture

Positive functional expression of HEK-293 cells stably expressing rat mGluR5 receptor was determined by measuring intracellular Ca²⁺ changes using a Fluorometric Imaging Plate Reader (FLIPR, Molecular Devices, Sunnyvale, Calif.) in response to glutamate or selective known mGluR5 agonists and antagonists. Rat mGluR5 RT-PCR products in HEK-293 cells were sequenced and found 100% identical to rat mGluR5 Genbank reference sequence (NM_{—}017012). HEK-293 cells expressing rmGluR5 were maintained in media containing DMEM, dialyzed Fetal Bovine Serum (10%), Glutamax™ (2 mM), Penicillin (100 units/ml), Streptomycin (100 μg/ml), Geneticin (100 μg/ml) and Hygromycin-B (40 μg/ml) at 37° C./5% CO2.

### Fluorescent Cell Based-Ca²⁺ Mobilization Assay

After one day of incubation, cells were washed with assay buffer containing: 142 mM NaCl, 6 mM KCl, 1 mM Mg₂SO₄, 1 mM CaCl₂, 20 mM HEPES, 1 g/L glucose, 0.125 mM sulfinpyrazone, pH 7.4. After 60 min of loading with 4 uM Fluo-4 (TefLabs, Austin, Tex.), the cells were washed three times with 50 μl of PBS Buffer and resuspended in 45 μl of assay Buffer. The plates were then transferred to a Fluorometric Imaging Plate Reader (FLIPR, Molecular Devices, Sunnyvale, Calif.) for the assessment of intracellular calcium flux. After monitoring the baseline fluorescence for 10 seconds, increasing concentrations of representative compound (from 0.01 to 60 μM) of the present invention diluted in Assay Buffer (15 μl of 4× dilutions) was added to the cell. The final DMSO concentration in the assay was 0.3%. In each experiment, fluorescence was then monitored as a function of time for 3 minutes and the data analyzed using Microsoft Excel and GraphPad Prism. Each data point was also measured two times.

Under these experimental conditions, this HEK-rat mGluR5 cell line is able to directly detect positive allosteric modulators without the need of co-addition of glutamate or mGluR5 agonist. Thus, DFB, CPPHA and CDPPB, published reference positive allosteric modulators that are inactive in rat cortical astrocytes culture in the absence of added glutamate (Liu et al (2006) Eur. J. Pharmacol. 536:262-268; Zhang et al (2005); J. Pharmacol. Exp. Ther. 315:1212-1219) are activating, in this system, rat mGluR5 receptors.

The concentration-response curves of representative compounds of the present invention were generated using the Prism GraphPad software (Graph Pad Inc, San Diego, USA). The curves were fitted to a four-parameter logistic equation:

(Y=Bottom+(Top−Bottom)/(1+10̂((LogEC₅₀−X)*Hill Slope)

allowing determination of EC₅₀ values.

The Table 1 below represents the mean EC₅₀ obtained from at least three independent experiments of selected molecules performed in duplicate.

TABLE 1

| EXAMPLE | Ca++ Flux* |
|---|---|
| 1 | +++ |
| 2 | +++ |
| 3 | +++ |
| 4 | ++ |
| 5 | +++ |
| 6 | +++ |
| 7 | +++ |
| 8 | ++ |
| 9 | ++ |
| 10 | +++ |
| 11 | ++ |
| 12 | ++ |
| 13 | + |
| 14 | + |
| 15 | + |
| 16 | ++ |
| 17 | ++ |
| 18 | ++ |
| 19 | ++ |
| 20 | + |
| 21 | +++ |
| 22 | +++ |
| 23 | ++ |
| 24 | ++ |
| 25 | ++ |
| 26 | ++ |
| 27 | +++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | ++ |
| 38 | ++ |
| 39 | +++ |
| 40 | +++ |
| 41 | +++ |
| 42 | +++ |
| 43 | +++ |
| 44 | +++ |
| 44 | + |
| 45 | +++ |
| 46 | +++ |
| 47 | ++ |
| 49 | ++ |
| 50 | +++ |
| 51 | +++ |
| 52 | +++ |
| 53 | +++ |
| 54 | +++ |
| 55 | ++ |
| 57 | ++ |
| 58 | +++ |
| 60 | +++ |
| 64 | ++ |
| 65 | ++ |
| 66 | ++ |
| 67 | ++ |
| 68 | ++ |
| 69 | ++ |
| 70 | ++ |
| 71 | ++ |
| 72 | ++ |
| 76 | + |
| 77 | ++ |
| 79 | ++ |

*Table legend:
(+): EC₅₀ > 10 μM
(++): 1 μM < EC₅₀ < 10 μM
(+++): EC₅₀ < 1 μM

### Example C

### mGluR5 Binding Assay

Activity of compounds of the invention was examined following a radioligand binding technique using whole rat brain and tritiated 2-methyl-6-(phenylethynyl)-pyridine ([³H]-MPEP) as a ligand following similar methods than those described in Gasparini et al. (2002) Bioorg. Med. Chem. Lett. 12:407-409 and in Anderson et al. (2002) J. Pharmacol. Exp. Ther. 303 (3) 1044-1051.

### Membrane Preparation:

Cortices were dissected out from brains of 200-300g Sprague-Dawley rats (Charles River Laboratories, L'Arbresle, France). Tissues were homogenized in 10 volumes (vol/wt) of ice-cold 50 mM HEPES-NaOH (pH 7.4) using a Polytron disrupter (Kinematica AG, Luzern, Switzerland) and centrifuged for 30 min at 40,000 g. (4° C.). The supernatant was discarded and the pellet washed twice by resuspension in 10 volumes 50 mM HEPES-NaOH. Membranes were then collected by centrifugation and washed before final resuspension in 10 volumes of 20 mM HEPES-NaOH, pH 7.4. Protein concentration was determined by the Bradford method (Bio-Rad protein assay, Reinach, Switzerland) with bovine serum albumin as standard.

### [3H]-MPEP Binding Experiments:

Membranes were thawed and resuspended in binding buffer containing 20 mM HEPES-NaOH, 3 mM MgCl₂, 3 mM CaCl₂, 100 mM NaCl, pH 7.4. Competition studies were carried out by incubating for 1 h at 4° C.: 3 nM [³H]-MPEP (39 Ci/mmol, Tocris, Cookson Ltd, Bristol, U.K.), 50 μg membrane and a concentration range of 0.003 nM-30 μM of compounds, for a total reaction volume of 300 μl. The non-specific binding was defined using 30 μM MPEP. Reaction was terminated by rapid filtration over glass-fiber filter plates (Unifilter 96-well GF/B filter plates, Perkin-Elmer, Schwerzenbach, Switzerland) using 4×400 μl ice cold buffer using cell harvester (Filtermate, Perkin-Elmer, Downers Grove, USA). Radioactivity was determined by liquid scintillation spectrometry using a 96-well plate reader (TopCount, Perkin-Elmer, Downers Grove, USA).

### Data Analysis:

The inhibition curves were generated using the Prism GraphPad program (Graph Pad Software Inc, San Diego, USA). IC₅₀ determinations were made from data obtained from 8 point-concentration response curves using a non linear regression analysis. The mean of IC₅₀ obtained from at least three independent experiments of selected molecules performed in duplicate were calculated.

The compounds of this application have IC₅₀ values in the range of less than 100 μM. Example #1 has IC₅₀ value of less than 30 μM.

The results shown in Examples A, B and C demonstrate that the compounds described in the present invention are positive allosteric modulators of rat mGluR5 receptors. These compounds are active in native systems and are able to inhibit the binding of the prototype mGluR5 allosteric modulator [³H]-MPEP known to bind remotely from the glutamate binding site into the transmembrane domains of mGluR5 receptors (Malherbe et al (2003) Mol. Pharmacol. 64 (4):823-32)

Thus, the positive allosteric modulators provided in the present invention are expected to increase the effectiveness of glutamate or mGluR5 agonists at mGluR5 receptor. Therefore, these positive allosteric modulators are expected to be useful for treatment of various neurological and psychiatric disorders associated with glutamate dysfunction described to be treated herein and others that can be treated by such positive allosteric modulators.

### Example D

### Amphetamine Model of Schizophrenia

Amphetamine-induced increases in locomotor ambulation are well known and are widely used as a model of the positive symptoms of schizophrenia. This model is based on evidence that amphetamine increases motor behaviors and can induce a psychotic state in humans (Yui et al. (2000) Ann. N.Y. Acad. Sci. 914:1-12). Further, it is well known that amphetamine-induced increases in locomotor activity are blocked by antipsychotics drugs that are effective in the treatment of schizophrenia (Arnt (1995) Eur. J. Pharmacol. 283:55-62). These results demonstrate that locomotor activation induced by amphetamine is a useful model for screening of compounds which may be useful in the treatment of schizophrenia.

Subjects: The present studies were performed in accordance with the animal care and use policies of Addex Pharmaceuticals and the laws and directives of Switzerland governing the care and use of animals. Male C57BL6/j mice (20-30 g) 7 weeks of age at the time of delivery were group housed in a temperature and humidity controlled facility on a 12 hour light/dark cycle for at least 7 days before use. Mice had access to food and water ad libitum except during locomotor activity experiments.

Assessment of locomotor (ambulatory) activity: The effects of compounds on amphetamine-induced locomotor activation in mice were tested. Locomotor activity of mice was tested in white plastic boxes 35 cm×35 cm square with walls 40 cm in height. Locomotor activity (ambulations) was monitored by a videotracking system (VideoTrack, Viewpoint, Champagne au Mont d'Or, France) that recorded the ambulatory movements of mice. Mice were naïve to the apparatus prior to testing. On test days, test compounds (10, 30, 50 or 100 mg/kg i.p. (intraperitoneal)) or vehicle were administered 120 minutes before amphetamine (3.0 mg/kg s.c.) or saline injection. Mice were placed into the locomotor boxes immediately after amphetamine or saline vehicle injection and their locomotor activity, defined as the distance traveled in centimeters (cm), was measured for 60 minutes.

Compound administration: Compounds were prepared as a microsuspension in sterile water (60% of final volume) and Labrafil M1944 CS (apricot kernel oil—Gattefossé, Saint Priest, France) (40% of final volume) and administered in a volume of 10 ml/kg. Compound-vehicle-treated mice received the equivalent volume of vehicle solution i.p. in the absence of added compound. D-amphetaminie sulfate (Amino AG, Neuenhof, Switzerland) was dissolved in saline and administered at a dose of 3.0 mg/kg s.c. in a volume of 10 ml/kg. D-amphetamine-vehicle-treated mice received an equivalent volume of saline vehicle injected s.c.

Statistical analyses: Statistical analyses were performed using GraphPad PRISM statistical software (GraphPad, San Diego, Calif., USA). Data were analyzed using one-way analysis of variance (ANOVA) followed by post-hoc Bonferroni-corrected multiple comparisons, where appropriate. The significance level was set at p<0.05.

### Effect of Compounds on Amphetamine-Induced Locomotor Activity in Mice

Representative compound of the invention significantly attenuated the increase in locomotor activity induced by amphetamine.

The compounds of the present invention are allosteric modulators of mGluR5 receptors, they are useful for the production of medications, especially for the prevention or treatment of central nervous system disorders as well as other disorders modulated by this receptor.

The compounds of the invention can be administered either alone, or in combination with other pharmaceutical agents effective in the treatment of conditions mentioned above.

### Formulation Examples

Typical examples of recipes for the formulation of the invention are as follows:

1) Tablets

| | | |
|---|---|---|
| Compound of the example 1 | 5 to 50 | mg |
| Di-calcium phosphate | 20 | mg |
| Lactose | 30 | mg |
| Talcum | 10 | mg |
| Magnesium stearate | 5 | mg |
| Potato starch | ad 200 | mg |

In this example, the compound of the example 1 can be replaced by the same amount of any of the described examples 1 to 81.

2) Suspension

An aqueous suspension is prepared for oral administration so that each 1 milliliter contains 1 to 5 mg of one of the described example, 50 mg of sodium carboxymethyl cellulose, 1 mg of sodium benzoate, 500 mg of sorbitol and water ad 1 ml.

3) Injectable

A parenteral composition is prepared by stirring 1.5% by weight of active ingredient of the invention in 10% by volume propylene glycol and water.

4) Ointment

| | | |
|---|---|---|
| Compound of the example 1 | 5 to 1000 | mg |
| Stearyl alcohol | 3 | g |
| Lanoline | 5 | g |
| White petroleum | 15 | g |
| Water | ad 100 | g |

In this example, the compound 1 can be replaced by the same amount of any of the described examples 1 to 81.

Reasonable variations are not to be regarded as a departure from the scope of the invention. It will be obvious that the thus described invention may be varied in many ways by those skilled in the art.

1. A compound which conforms to the general formula I: 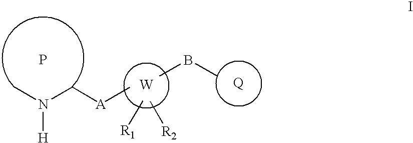
Wherein
W represents (C₄-C₇)cycloalkyl, (C₃-C₇)heterocycloalkyl, (C₃-C₇)heterocycloalkyl-(C₁-C₃)alkyl or (C₃-C₇)heterocycloalkenyl ring;
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula 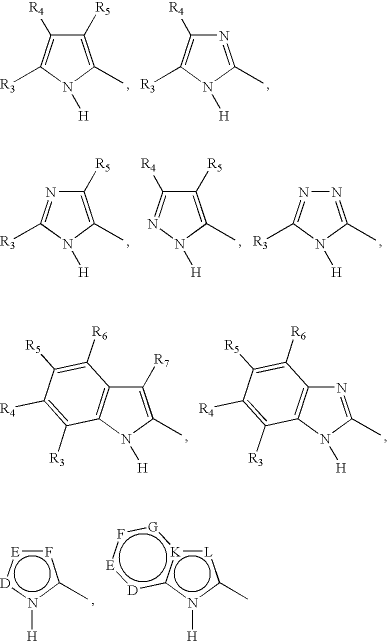
R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula 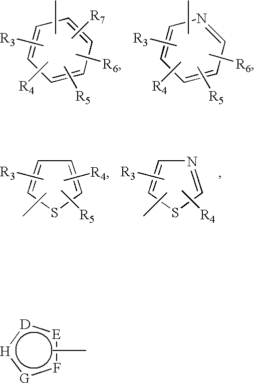
R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
A is azo —N═N—, ethyl, ethenyl, ethynyl, —NR₈C(═O)—, —NR₈C(═O)—O—, —NR₈C(═O)—NR₉, NR₈S(═O)₂—, —C(═O)NR₈—, —O—C(═O)NR₈—, —S—, —S(═O)—, —S(═O)₂—, —S(═O)₂NR₈—, —C(═O)—O—, —O—C(═O)—, —C(═NR₈)NR₉—, C(═NOR₈)NR₉—, —NR₈C(═NOR₉)—, ═N—O—, —O—N═CH— or a group aryl or heteroaryl of formula 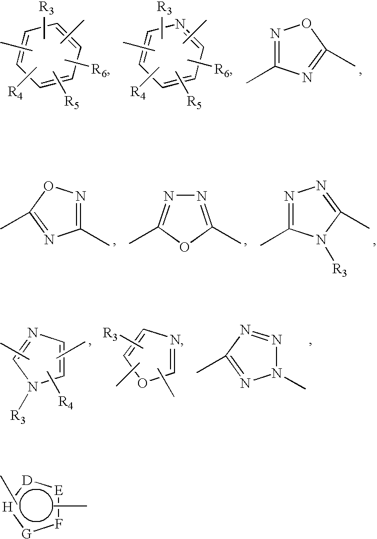
R₃, R₄, R₅ and R₆ independently are as defined above;
D, E, F, G and H in A independently represent —C(R₃)═, —C(R₃)═C(R₄)—, —C(═O)—, —C(═S)—, —O—, —N═, —N(R₃)— or —S—;
R₃, R₄, R₅ and R₆ independently are as defined above;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
Any N may be an N-oxide;
or pharmaceutically acceptable salts, hydrates or solvates of such compounds.

2. A compound according to claim 1 having the formula I-A 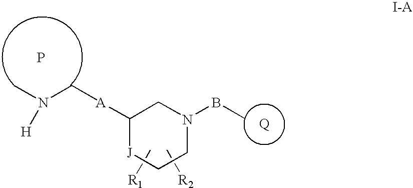
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula 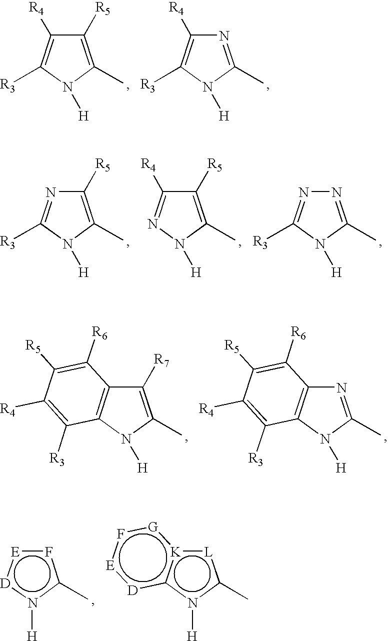
R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula 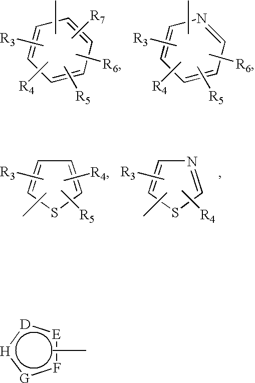
R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
A is azo —N═N—, ethyl, ethenyl, ethynyl, —NR₈C(═O)—, —NR₈C(═O)—O—, —NR₈C(═O)—NR₉, NR₈S(═O)₂—, —C(═O)NR₈—, —O—C(═O)NR₈—, —S—, —S(═O)—, —S(═O)₂—, —S(═O)₂NR₈—, —C(═O)—O—, —O—C(═O)—, —C(═NR₈)NR₉—, C(═NOR₈)NR₉—, —NR₈C(═NOR₉)—, ═N—O—, —O—N═CH— or a group aryl or heteroaryl of formula 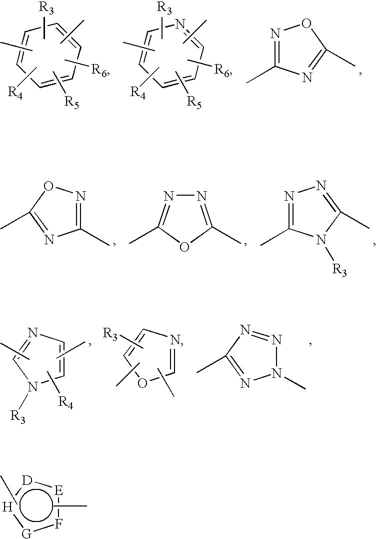
R₃, R₄, R₅ and R₆ independently are as defined above;
D, E, F, G and H in A independently represent —C(R₃)═, —C(R₃)═C(R₄)—, —C(═O)—, —C(═S)—, —O—, —N═, —N(R₃)— or —S—;
R₃, R₄, R₅ and R₆ independently are as defined above;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
J represents a single bond, —C(R₁₀, R₁₁), —O—, —N(R₁₀)— or —S—;
R₁₀, R₁₁ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

3. A compound according to claim 1 having the formula I-B 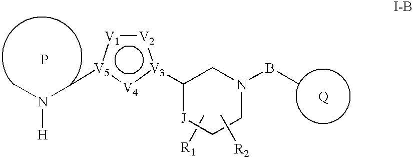
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula 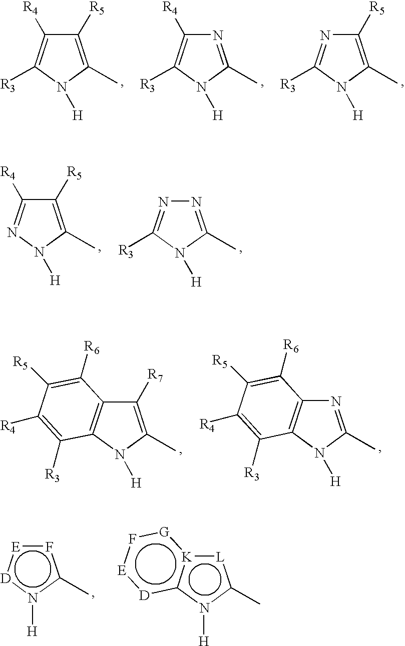
R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula 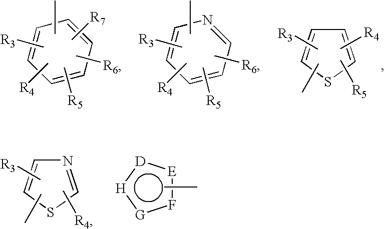
R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
V₁, V₂, V₃, V₄ and V₅ represent independently —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
J represents a single bond, —C(R₁₀, R₁₁), —O—, —N(R₁₀)— or —S—;
R₁₀, R₁₁ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

4. A compound according to claim 1 having the formula I-C 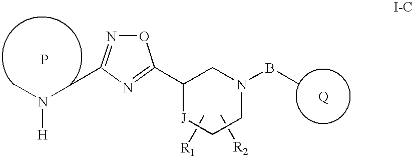
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula 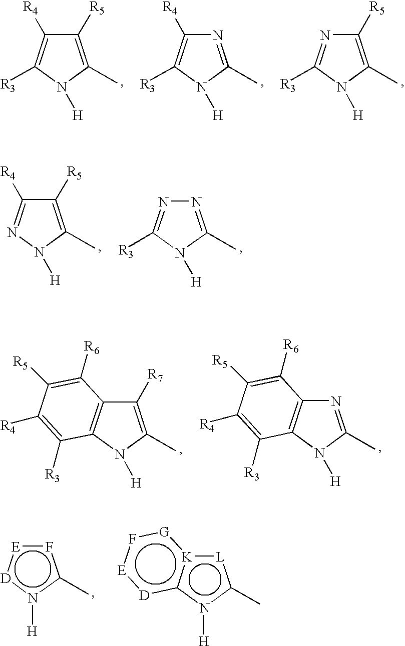
R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula 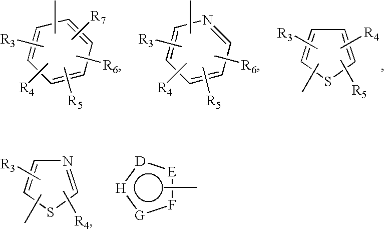
R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
J represents a single bond, —C(R₁₀, R₁₁), —O—, —N(R₁₀)— or —S—;
R₁₀, R₁₁, independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

5. A compound according to claim 1 having the formula I-D 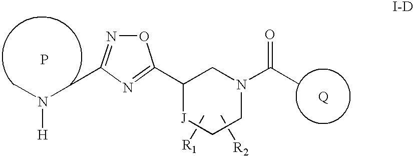
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula 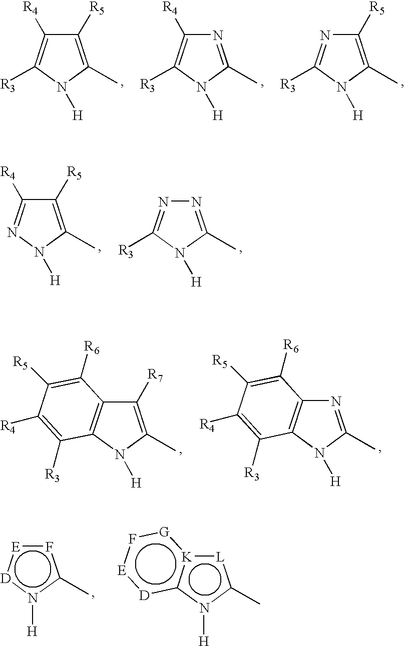
R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula 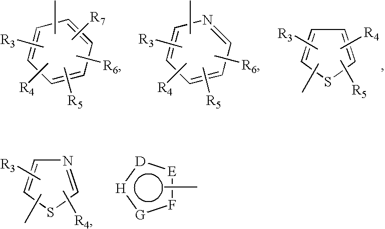
R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
J represents a single bond, —C(R₁₀, R₁₁), —O—, —N(R₁₀)— or —S—;
R₁₀, R₁₁, independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

6. A compound according to claim 1 having the formula II-A 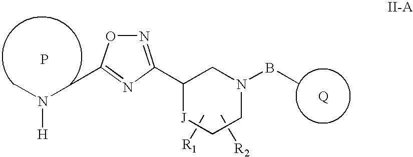
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula 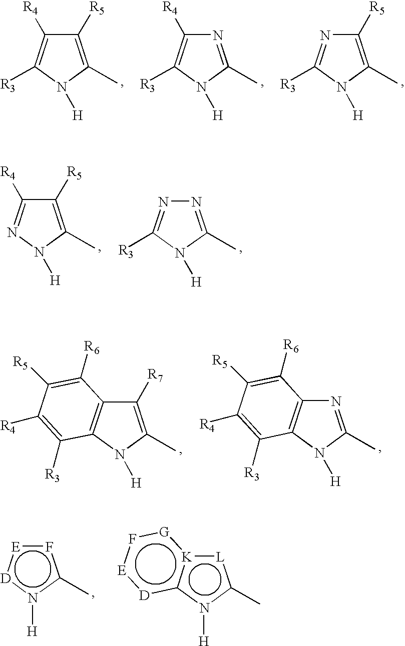
R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula 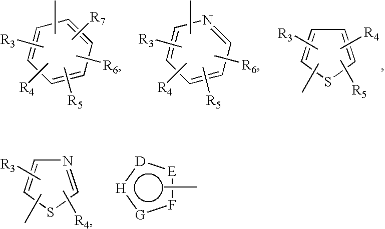
R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
B represents a single bond, —C(═O)—(C₀-C₂)alkyl-, —C(═O)—(C₂-C₆)alkenyl-, —C(═O)—(C₂-C₆)alkynyl-, —C(═O)—O—, —C(═O)NR₈—(C₀-C₂)alkyl-, —C(═NR₈)NR₉, —S(═O)—(C₀-C₂)alkyl-, —S(═O)₂—(C₀-C₂)alkyl-, —S(═O)₂NR₈—(C₀-C₂)alkyl-, C(═NR₈)—(C₀-C₂)alkyl-, —C(═NOR₈)—(C₀-C₂)alkyl- or —C(═NOR₈)NR₉—(C₀-C₂)alkyl-;
R₈ and R₉, independently are as defined above;
J represents a single bond, —C(R₁₀, R₁₁), —O—, —N(R₁₀)— or —S—;
R₁₀, R₁₁ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C6)alkyl)(aryl) substituents;
Any N may be an N-oxide;
or pharmaceutically acceptable salts, hydrates or solvates of such compounds.

7. A compound according to claim 1 having the formula II-B 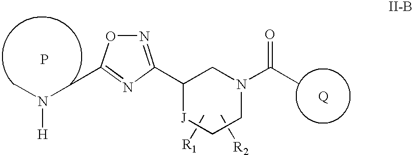
Wherein
R₁ and R₂ represent independently hydrogen, —(C₁-C₆)alkyl, —(C₂-C₆)alkenyl, —(C₂-C₆)alkynyl, arylalkyl, heteroarylalkyl, hydroxy, amino, aminoalkyl, hydroxyalkyl, —(C₁-C₆)alkoxy or R₁ and R₂ together can form a (C₃-C₇)cycloalkyl ring, a carbonyl bond C═O or a carbon double bond;
P represents a (C₅-C₇)heterocycloalkyl, (C₅-C₇)heterocycloalkenyl ring or a heteroaryl group of formula 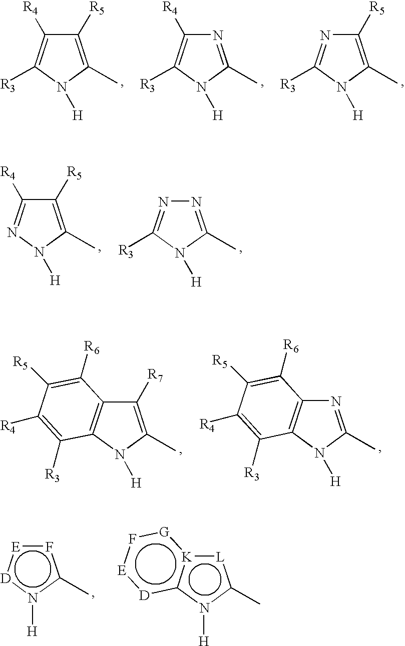
R₃, R₄, R₅, R₆, and R₇ independently are hydrogen, halogen, —NO₂, —(C₁-C₆)alkyl, —(C₃-C₆)cycloalkyl, —(C₃-C₇)cycloalkylalkyl, —(C₂-C₆)alkenyl, —(C₂-C₆)alkynyl, halo-(C₁-C₆)alkyl, heteroaryl, heteroarylalkyl, arylalkyl, aryl, —OR₈, —NR₈R₉, —C(═NR₁₀)NR₈R₉, —NR₈COR₉, NR₈CO₂R₉, NR₈SO₂R₉, —NR₁₀CO NR₈R₉, —SR₈, —S(═O)R₈, —S(═O)₂R₈, —S(═O)₂NR₈R₉, —C(═O)R₈, —C(O)—O—R₈, —C(═O)NR₈R₉, —C(═NR₈)R₉, or C(═NOR₈)R₉ substituents; wherein optionally two substituents are combined to the intervening atoms to form a bicyclic heterocycloalkyl, aryl or heteroaryl ring; wherein each ring is optionally further substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —O—(—C₁-C₃)alkylaryl, —O—(C₁-C₃)alkylheteroaryl, —N((—C₀-C₆)alkyl)((C₀-C₃)alkylaryl) or —N((C₀-C₆)alkyl)((C₀-C₃—)alkylheteroaryl) groups;
R₈, R₉, R₁₀ each independently is hydrogen, (C₁-C₆)alkyl, (C₃-C₆)cycloalkyl, (C₃-C₇)cycloalkylalkyl, (C₂-C₆)alkenyl, (C₂-C₆)alkynyl, halo-(C₁-C₆)alkyl, heterocycloalkyl, heteroaryl, heteroarylalkyl, arylalkyl or aryl; any of which is optionally substituted with 1-5 independent halogen, —CN, —(C₁-C₆)alkyl, —O—(C₀-C₆)alkyl, —O—(C₃-C₇)cycloalkylalkyl, —O(aryl), —O(heteroaryl), —N(C₀-C₆-alkyl)₂, —N((C₀-C₆)alkyl)((C₃-C₇—)cycloalkyl) or —N((C₀-C₆)alkyl)(aryl) substituents;
D, E, F, G, K and L in P independently represent —C(R₃)═, —C(R₃)═C(R₄)—, —C(═O)—, —C(═S)—, —O—, —N═, —N(R₃)— or —S—;
Q denotes a cycloalkyl, an aryl or heteroaryl group of formula 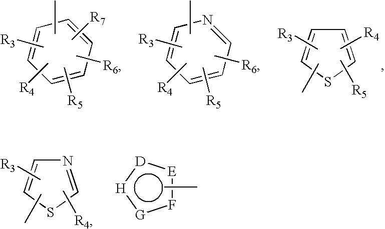
R₃, R₄, R₅, R₆, and R₇ independently are as defined above;
D, E, F, G and H in Q independently represent —C(R₃)═, —C(R₃)═C(R₄)—, —C(═O)—, —C(═S)—, —O—, —N═, —N(R₃)— or —S—;
J represents a single bond, —C(R₁₀, R₁₁), —O—, —N(R₁₀)— or —S—;
R₁₀, R₁₁ independently are hydrogen, —(C₁-C₆)alkyl, —(C₃-C₆)cycloalkyl, —(C₃-C₇)cycloalkylalkyl, —(C₂-C₆)alkenyl, —(C₂-C₆)alkynyl, halo(C₁-C₆)alkyl, heteroaryl, heteroarylalkyl, arylalkyl or aryl; any of which is optionally substituted with 1-5 independent halogen, —CN, —(C₁-C₆)alkyl, —O(C₀-C₆)alkyl, —O(C₃-C₇)cycloalkylalkyl, —O(aryl), —O(heteroaryl), —N((C₀-C₆)alkyl)((C₀-C₆)alkyl), —N((C₀-C₆)alkyl)((C₃-C₇)cycloalkyl) or —N((C₀-C₆)alkyl)(aryl) substituents;
Any N may be an N-oxide;
or pharmaceutically acceptable salts, hydrates or solvates of such compounds.

8. A compound according to claim 1, which can exist as optical isomers, wherein said compound is either the racemic mixture or an individual optical isomer.

9. A compound according to claim 1, wherein said compound is selected from:
(4-Fluoro-phenyl)-{(S)-3-[3-(1H-pyrrol-2-yl)-[1,2,4]oxadiazol-5-yl]-piperidin-1-yl}-methanone
(2,4-Difluoro-phenyl)-{(S)-3-[3-(1H-pyrrol-2-yl)-[1,2,4]oxadiazol-5-yl]-piperidin-1-yl}-methanone
(3,4-Difluoro-phenyl)-{(S)-3-[3-(1H-pyrrol-2-yl)-[1,2,4]oxadiazol-5-yl]-piperidin-1-yl}-methanone
(6-Fluoro-pyridin-3-yl)-{(S)-3-[3-(1H-pyrrol-2-yl)-[1,2,4]oxadiazol-5-yl]-piperidin-1-yl}-methanone
(3,4-Difluoro-phenyl)-{3-[5-(1H-pyrrol-2-yl)-[1,2,4]oxadiazol-3-yl]-piperidin-1-yl}-methanone
(2,4-Difluoro-phenyl)-{3-[5-(1H-pyrrol-2-yl)-[1,2,4]oxadiazol-3-yl]-piperidin-1-yl}-methanone
(4-Fluoro-phenyl)-{3-[5-(1H-pyrrol-2-yl)-[1,2,4]oxadiazol-3-yl]-piperidin-1-yl}-methanone
(6-Fluoro-pyridin-3-yl)-{3-[5-(1H-pyrrol-2-yl)-[1,2,4]oxadiazol-3-yl]-piperidin-1-yl}-methanone
(4-Fluoro-2-methyl-phenyl)-{3-[5-(1H-pyrrol-2-yl)-[1,2,4]oxadiazol-3-yl]-piperidin-1-yl}-methanone
(3,4-Difluoro-phenyl)-{(S)-3-[5-(1H-pyrrol-2-yl)-[1,2,4]oxadiazol-3-yl]-piperidin-1-yl}-methanone
(4-Fluoro-phenyl)-{3-[5-(1H-indol-2-yl)-[1,2,4]oxadiazol-3-yl]-piperidin-1-yl}-methanone
(2,4-Difluoro-phenyl)-{(S)-3-[3-(1H-indol-2-yl)-[1,2,4]oxadiazol-5-yl]-piperidin-1-yl}-methanone
(4-Fluoro-phenyl)-{3-[5-(2H-pyrazol-3-yl)-[1,2,4]oxadiazol-3-yl]-piperidin-1-yl}-methanone
(3,4-Difluoro-phenyl)-{3-[5-(2H-pyrazol-3-yl)-[1,2,4]oxadiazol-3-yl]-piperidin-1-yl}-methanone
(4-Fluoro-phenyl)-{3-[5-(1H-imidazol-2-yl)-[1,2,4]oxadiazol-3-yl]-piperidin-1-yl}-methanone
(3,4-Difluoro-phenyl)-{(S)-3-[3-(1H-indol-2-yl)-[1,2,4]oxadiazol-5-yl]-piperidin-1-yl}-methanone
(4-Fluoro-phenyl)-{(S)-3-[3-(1H-indol-2-yl)-[1,2,4]oxadiazol-5-yl]-piperidin-1-yl}-methanone
(3,4-diFluoro-phenyl)-{3-[5-(1H-imidazol-2-yl)-[1,2,4]oxadiazol-3-yl]-piperidin-1-yl}-methanone.
{(S)-3-[3-(1H-Indol-2-yl)-[1,2,4]oxadiazol-5-yl]-piperidin-1-yl}-(5-methyl-isoxazol-4-yl)-methanone
(5-Methyl-isoxazol-4-yl)-{(S)-3-[3-(1H-pyrrol-2-yl)-[1,2,4]oxadiazol-5-yl]-piperidin-1-yl}-methanone
(6-Fluoro-pyridin-3-yl)-{(S)-3-[5-(1H-pyrrol-2-yl)-[1,2,4]oxadiazol-3-yl]-piperidin-1-yl}-methanone
(4-Fluoro-phenyl)-{(S)-3-[5-(1H-pyrrol-2-yl)-[1,2,4]oxadiazol-3-yl]-piperidin-1-yl}-methanone
(6-Fluoro-pyridin-3-yl)-{3-[5-(1H-indol-2-yl)-[1,2,4]oxadiazol-3-yl]-piperidin-1-yl}-methanone
(4-Fluoro-phenyl)-{(S)-3-[3-(1H-imidazol-2-yl)-[1,2,4]oxadiazol-5-yl]-piperidin-1-yl}-methanone
(3,4-Difluoro-phenyl)-{(S)-3-[3-(1H-imidazol-2-yl)-[1,2,4]oxadiazol-5-yl]-piperidin-1-yl}-methanone
{3-[5-(1H-Indol-2-yl)-[1,2,4]oxadiazol-3-yl]-piperidin-1-yl}-(5-methyl-isoxazol-4-yl)-methanone
(4-Fluoro-phenyl)-{(S)-3-[5-(4-methyl-1H-pyrrol-2-yl)-[1,2,4]oxadiazol-3-yl]-piperidin-1-yl}-methanone
(6-Fluoro-pyridin-3-yl)-{(S)-3-[5-(4-methyl-1H-pyrrol-2-yl)-[1,2,4]oxadiazol-3-yl]-piperidin-1-yl}-methanone
(5-Methyl-isoxazol-4-yl)-{(S)-3-[5-(4-methyl-1H-pyrrol-2-yl)-[1,2,4]oxadiazol-3-yl]-piperidin-1-yl}-methanone
(2-Fluoro-pyridin-4-yl)-{(S)-3-[5-(4-methyl-1H-pyrrol-2-yl)-[1,2,4]oxadiazol-3-yl]-piperidin-1-yl}-methanone
(4-Fluoro-phenyl)-{(S)-3-[3-(4-methyl-1H-pyrrol-2-yl)-[1,2,4]oxadiazol-5-yl]-piperidin-1-yl}-methanone
(3,4-Difluoro-phenyl)-{(S)-3-[3-(4-methyl-1H-pyrrol-2-yl)-[1,2,4]oxadiazol-5-yl]-piperidin-1-yl}-methanone
(6-Fluoro-pyridin-3-yl)-{(S)-3-[3-(4-methyl-1H-pyrrol-2-yl)-[1,2,4]oxadiazol-5-yl]-piperidin-1-yl}-methanone
(2-Fluoro-pyridin-4-yl)-{(S)-3-[3-(4-methyl-1H-pyrrol-2-yl)-[1,2,4]oxadiazol-5-yl]-piperidin-1-yl}-methanone
(5-Methyl-isoxazol-4-yl)-{(S)-3-[3-(4-methyl-1H-pyrrol-2-yl)-[1,2,4]oxadiazol-5-yl]-piperidin-1-yl}-methanone
(4-Fluoro-phenyl)-{(S)-3-[5-(4-nitro-1H-pyrrol-2-yl)-[1,2,4]oxadiazol-3-yl]-piperidin-1-yl}-methanone
(4-Fluoro-phenyl)-{(R)-3-[3-(1H-pyrrol-2-yl)-[1,2,4]oxadiazol-5-yl]-piperidin-1-yl}-methanone
(4-Fluoro-phenyl)-{(S)-3-[5-(5-methyl-1H-pyrrol-2-yl)-[1,2,4]oxadiazol-3-yl]-piperidin-1-yl}-methanone
{(S)-3-[5-(4-Chloro-1H-pyrrol-2-yl)-[1,2,4]oxadiazol-3-yl]-piperidin-1-yl}-(4-fluoro-phenyl)-methanone
{(S)-3-[5-(4-Chloro-1H-pyrrol-2-yl)-[1,2,4]oxadiazol-3-yl]-piperidin-1-yl}-(6-fluoro-pyridin-3-yl)-methanone
{(S)-3-[5-(4-Chloro-1H-pyrrol-2-yl)-[1,2,4]oxadiazol-3-yl]-piperidin-1-yl}-(2-fluoro-pyridin-4-yl)-methanone
{(S)-3-[5-(4-Chloro-1H-pyrrol-2-yl)-[1,2,4]oxadiazol-3-yl]-piperidin-1-yl}-(5-methyl-isoxazol-4-yl)-methanone
{(S)-3-[3-(4-Chloro-1H-pyrrol-2-yl)-[1,2,4]oxadiazol-5-yl]-piperidin-1-yl}-(4-fluoro-phenyl)-methanone
{(S)-3-[5-(4-Bromo-1H-pyrrol-2-yl)-[1,2,4]oxadiazol-3-yl]-piperidin-1-yl}-(6-fluoro-pyridin-3-yl)-methanone
{(S)-3-[3-(4-Bromo-1H-pyrrol-2-yl)-[1,2,4]oxadiazol-5-yl]-piperidin-1-yl}-(4-fluoro-phenyl)-methanone
{(S)-3-[3-(4-Bromo-1H-pyrrol-2-yl)-[1,2,4]oxadiazol-5-yl]-piperidin-1-yl}-(6-fluoro-pyridin-3-yl)-methanone
(4-Fluoro-phenyl)-{3-fluoro-3-[3-(1H-pyrrol-2-yl)-[1,2,4]oxadiazol-5-yl]-piperidin-1-yl}-methanone
{3,3-Difluoro-5-[3-(1H-pyrrol-2-yl)-[1,2,4]oxadiazol-5-yl]-piperidin-1-yl}-(4-fluoro-phenyl)-methanone
{3,3-Dimethyl-5-[3-(1H-pyrrol-2-yl)-[1,2,4]oxadiazol-5-yl]-piperidin-1-yl}-(4-fluoro-phenyl)-methanone
(4-Fluoro-phenyl)-{(S)-3-[3-(4-fluoro-1H-pyrrol-2-yl)-[1,2,4]oxadiazol-5-yl]-piperidin-1-yl}-methanone
(3,4-Difluoro-phenyl)-{(S)-3-[3-(4-fluoro-1H-pyrrol-2-yl)-[1,2,4]oxadiazol-5-yl]-piperidin-1-yl}-methanone
(6-Fluoro-pyridin-3-yl)-{(S)-3-[3-(4-fluoro-1H-pyrrol-2-yl)-[1,2,4]oxadiazol-5-yl]-piperidin-1-yl}-methanone
(2-Fluoro-pyridin-4-yl)-{(S)-3-[3-(4-fluoro-1H-pyrrol-2-yl)-[1,2,4]oxadiazol-5-yl]-piperidin-1-yl}-methanone
(4-Fluoro-phenyl)-{(S)-3-[5-(1H-pyrrol-2-yl)-tetrazol-2-yl]-piperidin-1-yl}-methanone
(4-Fluoro-phenyl)-{(S)-3-[5-(4-trifluoromethyl-1H-imidazol-2-yl)-[1,2,4]oxadiazol-3-yl]-piperidin-1-yl}-methanone
(6-Fluoro-pyridin-3-yl)-{(S)-3-[5-(4-isopropyl-1H-pyrrol-2-yl)-[1,2,4]oxadiazol-3-yl]-piperidin-1-yl}-methanone
(4-Fluoro-phenyl)-{3-[3-(1H-pyrrol-2-yl)-[1,2,4]oxadiazol-5-yl]-pyrrolidin-1-yl}-methanone
(3-Fluoro-pyridin-4-yl)-{(S)-3-[5-(4-methyl-1H-pyrrol-2-yl)-[1,2,4]oxadiazol-3-yl]-piperidin-1-yl}-methanone
{(S)-3-[5-(4-Chloro-1H-pyrrol-2-yl)-[1,2,4]oxadiazol-3-yl]-piperidin-1-yl}-(3-fluoro-pyridin-4-yl)-methanone
(2-Fluoro-pyridin-4-yl)-{(S)-3-[5-(4-fluoro-1H-pyrrol-2-yl)-[1,2,4]oxadiazol-3-yl]-piperidin-1-yl}-methanone
{(S)-3-[5-(4-Bromo-1H-pyrrol-2-yl)-[1,2,4]oxadiazol-3-yl]-piperidin-1-yl}-(3-fluoro-pyridin-4-yl)-methanone
(3-Fluoro-pyridin-4-yl)-{(S)-3-[5-(4-fluoro-1H-pyrrol-2-yl)-[1,2,4]oxadiazol-3-yl]-piperidin-1-yl}-methanone
(4-Fluoro-phenyl)-{(S)-3-[5-(4-fluoro-1H-pyrrol-2-yl)-[1,2,4]oxadiazol-3-yl]-piperidin-1-yl}-methanone
(6-Fluoro-pyridin-3-yl)-{(S)-3-[5-(4-fluoro-1H-pyrrol-2-yl)-[1,2,4]oxadiazol-3-yl]-piperidin-1-yl}-methanone
{(S)-3-[3-(4-Chloro-1H-pyrrol-2-yl)-[1,2,4]oxadiazol-5-yl]-piperidin-1-yl}-(6-fluoro-pyridin-3-yl)-methanone
{(S)-3-[3-(4-Chloro-1H-pyrrol-2-yl)-[1,2,4]oxadiazol-5-yl]-piperidin-1-yl}-(2-fluoro-pyridin-4-yl)-methanone
{(S)-3-[3-(4-Chloro-1H-pyrrol-2-yl)-[1,2,4]oxadiazol-5-yl]-piperidin-1-yl}-(3-fluoro-pyridin-4-yl)-methanone
{(S)-3-[3-(4-Chloro-1H-pyrrol-2-yl)-[1,2,4]oxadiazol-5-yl]-piperidin-1-yl}-(5-methyl-isoxazol-4-yl)-methanone
{(S)-3-[3-(4-Bromo-1H-pyrrol-2-yl)-[1,2,4]oxadiazol-5-yl]-piperidin-1-yl}-(3-fluoro-pyridin-4-yl)-methanone
(3-Fluoro-pyridin-4-yl)-{(S)-3-[3-(4-fluoro-1H-pyrrol-2-yl)-[1,2,4]oxadiazol-5-yl]-piperidin-1-yl}-methanone
(3-Fluoro-pyridin-4-yl)-{(S)-3-[3-(4-methyl-1H-pyrrol-2-yl)-[1,2,4]oxadiazol-5-yl]-piperidin-1-yl}-methanone
(4-Fluoro-phenyl)-{(S)-3-[5-(4-cyano-1H-pyrrol-2-yl)-[1,2,4]oxadiazol-3-yl]-piperidin-1-yl}-methanone
5-{3-[(S)-1-(6-Fluoro-pyridine-3-carbonyl)-piperidin-3-yl]-[1,2,4]oxadiazol-5-yl}-1H-pyrrole-3-carbonitrile
5-{3-[(S)-1-(2-Fluoro-pyridine-4-carbonyl)-piperidin-3-yl]-[1,2,4]oxadiazol-5-yl}-1H-pyrrole-3-carbonitrile
5-{3-[(S)-1-(3-Fluoro-pyridine-4-carbonyl)-piperidin-3-yl]-[1,2,4]oxadiazol-5-yl}-1H-pyrrole-3-carbonitrile
(4-Fluoro-phenyl)-{(S)-3-[5-(4-trifluoromethyl-1H-pyrrol-2-yl)-[1,2,4]oxadiazol-3-yl]-piperidin-1-yl}-methanone
(3-Fluoro-pyridin-4-yl)-{(S)-3-[5-(4-trifluoromethyl-1H-pyrrol-2-yl)[1,2,4]oxadiazol-3-yl]-piperidin-1-yl}-methanone
(6-Fluoro-pyridin-3-yl)-{(S)-3-[5-(4-trifluoromethyl-1H-pyrrol-2-yl)-[1,2,4]oxadiazol-3-yl]-piperidin-1-yl}-methanone
(3,4-Difluoro-phenyl)-{(S)-3-[3-(4-methyl-1H-imidazol-2-yl)-[1,2,4]oxadiazol-5-yl]-piperidin-1-yl}-methanone
{(S)-3-[5-(4-Chloro-1H-pyrrol-2-yl)-[1,2,4]oxadiazol-3-yl]-piperidin-1-yl}-pyridin-4-yl-methanone
(6-Fluoro-pyridin-3-yl)-{(S)-3-[3-(4-trifluoromethyl-1H-pyrrol-2-yl)-[1,2,4]oxadiazol-5-yl]-piperidin-1-yl}-methanone.

10. A pharmaceutical composition comprising a therapeutically effective amount of a compound according to claim 1 and a pharmaceutically acceptable carrier and/or excipient.

11. A method of treating or preventing a condition in a mammal, including a human, the treatment or prevention of which is affected or facilitated by the neuromodulatory effect of mGluR5 allosteric modulators, comprising administering to a mammal in need of such treatment or prevention, an effective amount of a compound according to claim 1.

12. A method of treating or preventing a condition in a mammal, including a human, the treatment or prevention of which is affected or facilitated by the neuromodulatory effect of mGluR5 positive allosteric modulators (enhancer), comprising administering to a mammal in need of such treatment or prevention, an effective amount of a compound according to claim 1.

13. A method useful for treating or preventing central nervous system disorders selected from the group consisting of anxiety disorders: Agoraphobia, Generalized Anxiety Disorder (GAD), Obsessive-Compulsive Disorder (OCD), Panic Disorder, Posttraumatic Stress Disorder (PTSD), Social Phobia, Other Phobias, Substance-induced Anxiety Disorder, comprising administering an effective amount of a compound according to claim 1.

14. A method useful for treating or preventing central nervous system disorders selected from the group consisting of childhood disorders: Attention-Deficit/Hyperactivity Disorder), comprising administering an effective amount of a compound according to claim 1.

15. A method useful for treating or preventing central nervous system disorders selected from the group consisting of eating Disorders (Anorexia Nervosa, Bulimia Nervosa), comprising administering an effective amount of a compound according to claim 1.

16. A method useful for treating or preventing central nervous system disorders selected from the group consisting of mood disorders: Bipolar Disorders (I & II), Cyclothymic Disorder, Depression, Dysthymic Disorder, Major Depressive Disorder, Substance-induced Mood Disorder, comprising administering an effective amount of a compound according to claim 1.

17. A method useful for treating or preventing central nervous system disorders selected from the group consisting of psychotic disorders: Schizophrenia, Delusional Disorder, Schizoaffective Disorder, Schizophreniform Disorder, Substance-Induced Psychotic Disorder, comprising administering an effective amount of a compound according to claim 1.

18. A method useful for treating or preventing central nervous system disorders selected from the group consisting of cognitive disorders: Delirium, Substance-induced Persisting Delirium, Dementia, Dementia Due to HIV Disease, Dementia Due to Huntington's Disease, Dementia Due to Parkinson's Disease, Dementia of the Alzheimer's Type, Substance-Induced Persisting Dementia, Mild Cognitive Impairment, comprising administering an effective amount of a compound according to claim 1.

19. A method useful for treating or preventing central nervous system disorders selected from the group consisting of personality disorders: Obsessive-Compulsive Personality Disorder, Schizoid, Schizotypal disorder, comprising administering an effective amount of a compound according to claim 1.

20. A method useful for treating or preventing central nervous system disorders selected from the group consisting of substance-related disorders: Alcohol abuse, Alcohol dependence, Alcohol withdrawal, Alcohol withdrawal delirium, Alcohol-induced psychotic disorder, Amphetamine dependence, Amphetamine withdrawal, Cocaine dependence, Cocaine withdrawal, Nicotine dependence, Nicotine withdrawal, Opioid dependence, Opioid withdrawal, comprising administering an effective amount of a compound according to claim 1.

21. A method useful for treating or preventing inflammatory central nervous system disorders selected from multiple sclerosis form such as benign multiple sclerosis, relapsing-remitting multiple sclerosis, secondary progressive multiple sclerosis, primary progressive multiple sclerosis, progressive-relapsing multiple sclerosis, comprising administering an effective amount of a compound according to claims 1.

22-23. (canceled)

24. A method of treating or preventing a condition in a mammal, including a human, the treatment or prevention of which is affected or facilitated by the neuromodulatory effect of mGluR5 allosteric modulators, comprising administering to a mammal in need of such treatment or prevention, an effective amount of a compound according to claim 9.

25. A method of treating or preventing a condition in a mammal, including a human, the treatment or prevention of which is affected or facilitated by the neuromodulatory effect of mGluR5 allosteric modulators, comprising administering to a mammal in need of such treatment or prevention, an effective amount of a compound according to claim 10.
